(19) United States
(12) Patent Application Publication
(10) Pub. No.: US 2003/0207430 A1
(43) Pub. Date: Nov. 6, 2003

(54) Human enzyme molecules

(76) Inventors: Y. Tom Tang, San Jose, CA (US); Dyung Aina M. Lu, San Jose, CA (US); Olga Bandman, Mountain View, CA (US); Henry Yue, Sunnyvale, CA (US); Yalda Azimzai, Castro Valley, CA (US); Neil Burford, Durham, CT (US); Preeti Lal, Santa Clara, CA (US); Mariah R. Baughn, San Leandro, CA (US)

Correspondence Address: Incyte Genomics Inc, Legal Department, 3160 Porter Drive, Palo Alto, CA, 94304, US

(21) Appl. No.: 10/220,381

(22) PCT Filed: Mar. 1, 2001
(86) PCT No.: PCT/US01/06806

(51) Int. Cl. C12Q 1/68; A01K 67/00; C07H 21/04; C12N 9/00; C12P 21/02; C12N 5/06; C07K 16/40

(52) U.S. Cl. 435/183; 435/6; 435/69.1; 435/320.1; 435/325; 530/388.26; 536/23.2; 800/8

(57) ABSTRACT

The invention provides human enzyme molecules (HEM) and polynucleotides which identify and encode HEM. The invention also provides expression vectors, host cells, antibodies, agonists, and antagonists. The invention also provides methods for diagnosing, treating, or preventing disorders associated with aberrant expression of HEM.

### TECHNICAL FIELD

This invention relates to nucleic acid and amino acid sequences of human enzyme molecules and to the use of these sequences in the diagnosis, treatment, and prevention of autoimmune/inflammation disorders, genetic disorders, neurological disorders, and cell proliferative disorders including cancer, and in the assessment of the effects of exogenous compounds on the expression of nucleic acid and amino acid sequences of human enzyme molecules.

### BACKGROUND OF THE INVENTION

The cellular processes of biogenesis and biodegradation involve a number of key enzyme classes including oxidoreductases, transferases, hydrolases, lyases, isomerases, ligases, and cofactor biosynthetic enzymes. These enzyme classes are each comprised of numerous substrate-specific enzymes having precise and well regulated functions. These enzymes function by facilitating metabolic processes such as glycolysis, the tricarboxylic cycle, and fatty acid metabolism; synthesis or degradation of amino acids, steroids, phospholipids, alcohols, etc.; regulation of cell signalling, proliferation, inflamation, apoptosis, etc., and through catalyzing critical steps in DNA replication and repair, and the process of translation.

Oxidoreductases

Many pathways of biogenesis and biodegradation require oxidoreductase (dehydrogenase or reductase) activity, coupled to the reduction or oxidation of a donor or acceptor cofactor. Potential cofactors include cytochromes, oxygen, disulfide, iron-sulfur proteins, flavin adenine dinucleotide (FAD), and the nicotinamide adenine dinucleotides NAD and NADP (Newsholme, E. A. and Leech, A. R. (1983) Biochemistry for the Medical Sciences, John Wiley and Sons, Chichester, U.K. pp. 779-793). Reductase activity catalyzes the transfer of electrons between substrate(s) and cofactor(s) with concurrent oxidation of the cofactor. The reverse dehydrogenase reaction catalyzes the reduction of a cofactor and consequent oxidation of the substrate. Oxidoreductase enzymes are a broad superfamily of proteins that catalyze numerous reactions in all cells of organisms ranging from bacteria to plants to humans. These reactions include metabolism of sugar, certain detoxification reactions in the liver, and the synthesis or degradation of fatty acids, amino acids, glucocorticoids, estrogens, androgens, and prostaglandins. Different family members are named according to the direction in which their reactions are typically catalyzed; thus they may be referred to as oxidoreductases, oxidases, reductases, or dehydrogenases. In addition, family members often have distinct cellular localizations, including the cytosol, the plasma membrane, mitochondrial inner or outer membrane, and peroxisomes.

Short-chain alcohol dehydrogenases (SCADs) are a family of dehydrogenases that only share 15% to 30% sequence identity, with similarity predominantly in the coenzyme binding domain and the substrate binding domain. In addition to the well-known role in detoxification of ethanol, SCADs are also involved in synthesis and degradation of fatty acids, steroids, and some prostaglandins, and are therefore implicated in a variety of disorders such as lipid storage disease, myopathy, SCAD deficiency, and certain genetic disorders. For example, retinol dehydrogenase is a SCAD-family member (Simon, A. et al. (1995) J. Biol. Chem. 270:1107-1112) that converts retinol to retinal, the precursor of retinoic acid. Retinoic acid, a regulator of differentiation and apoptosis, has been shown to down-regulate genes involved in cell proliferation and inflammation (Chai, X. et al. (1995) J. Biol. Chem. 270:3900-3904). In addition, retinol dehydrogenase has been linked to hereditary eye diseases such as autosomal recessive childhood-onset severe retinal dystrophy (Simon, A. et al. (1996) Genomics 36:424-430).

Propagation of nerve impulses, modulation of cell proliferation and differentiation, induction of the immune response, and tissue homeostasis involve neurotransmitter metabolism (Weiss, B. (1991) Neurotoxicology 12:379-386; Collins, S. M. et al. (1992) Ann. N.Y. Acad. Sci. 664:415-424; Brown, J. K. and Imam, H. (1991) J. Inherit. Metab. Dis. 14:436-458). Many pathways of neurotransmitter metabolism require oxidoreductase activity, coupled to reduction or oxidation of a cofactor, such as NAD⁺/NADH (Newsholme, E. A. and Leech, A. R. (1983) Biochemistry for the Medical Sciences, John Wiley and Sons, Chichester, U.K. pp. 779-793). Degradation of catecholamines (epinephrine or norepinephrine) requires alcohol dehydrogenase (in the brain) or aldehyde dehydrogenase (in peripheral tissue). NAD⁺-dependent aldehyde dehydrogenase oxidizes 5-hydroxyindole-3-acetate (the product of 5-hydroxytryptamine (serotonin) metabolism) in the brain, blood platelets, liver and pulmonary endothelium (Newsholme, E. A. and Leech, A. R. (supra) p. 786). Other neurotransmitter degradation pathways that utilize NAD⁺/NADH-dependent oxidoreductase activity include those of L-DOPA (precursor of dopamine, a neuronal excitatory compound), glycine (an inhibitory neurotransmitter in the brain and spinal cord), histamine (liberated from mast cells during the inflammatory response), and taurine (an inhibitory neurotransmitter of the brain stem, spinal cord and retina) (Newsholme, E. A. and Leech, A. R. supra, pp. 790, 792). Epigenetic or genetic defects in neurotransmitter metabolic pathways can result in a spectrum of disease states in different tissues including Parkinson disease and inherited myoclonus (McCance, K. L. and Huether, S. E. (1994) Pathophysiology, Mosby-Year Book, Inc., St. Louis, Mo. pp. 402-404; Gundlach, A. L. (1990) FASEB J. 4:2761-2766).

Tetrahydrofolate is a derivatized glutamate molecule that acts as a carrier, providing activated one-carbon units to a wide variety of biosynthetic reactions, including synthesis of purines, pyrimidines, and the amino acid methionine. Tetrahydrofolate is generated by the activity of a holoenzyme complex called tetrahydrofolate synthase, which includes three enzyme activities: tetrahydrofolate dehydrogenase, tetrahydrofolate cyclohydrolase, and tetrahydrofolate synthetase. Thus, tetrahydrofolate dehydrogenase plays an important role in generating building blocks for nucleic and amino acids, crucial to proliferating cells.

3-Hydroxyacyl-CoA dehydrogenase (3HACD) is involved in fatty acid metabolism. It catalyzes the reduction of 3-hydroxyacyl-CoA to 3-oxoacyl-CoA, with concomitant oxidation of NAD to NADH, in the mitochondria and peroxisomes of eukaryotic cells. In peroxisomes, 3HACD and enoyl-CoA hydratase form an enzyme complex called bifunctional enzyme, defects in which are associated with peroxisomal bifunctional enzyme deficiency. This interruption in fatty acid metabolism produces accumulation of very-long chain fatty acids, disrupting development of the brain, bone, and adrenal glands. Infants born with this deficiency typically die within 6 months (Watkins, P. et al. (1989) J. Clin. Invest. 83:771-777; Online Mendelian Inheritance in Man (OMIM), #261515). The neurodegeneration that is characteristic of Alzheimer's disease involves development of extracellular plaques in certain brain regions. A major protein component of these plaques is the peptide amyloid-β (Aβ), which is one of several cleavage products of amyloid precursor protein (APP). 3HACD has been shown to bind the Aβ peptide, and is overexpressed in neurons affected in Alzheimer's disease. In addition, an antibody against 3HACD can block the toxic effects of Aβ in a cell culture model of Alzheimer's disease (Yan, S. et al. (1997) Nature 389:689-695; OMIM, #602057).

Steroids, such as estrogen, testosterone, corticosterone, and others, are generated from a common precursor, cholesterol, and are interconverted into one another. A wide variety of enzymes act upon cholesterol, including a number of dehydrogenases. Steroid dehydrogenases, such as the hydroxysteroid dehydrogenases, are involved in hypertension, fertility, and cancer (Duax, W. L. and Ghosh, D. (1997) Steroids 62:95-100). One such dehydrogenase is 3-oxo-5-α-steroid dehydrogenase (OASD), a microsomal membrane protein highly expressed in prostate and other androgen-responsive tissues. OASD catalyzes the conversion of testosterone into dihydrotestosterone, which is the most potent androgen. Dihydrotestosterone is essential for the formation of the male phenotype during embryogenesis, as well as for proper androgen-mediated growth of tissues such as the prostate and male genitalia. A defect in OASD that prevents the conversion of testosterone into dihydrotestosterone leads to a rare form of male pseudohermaphroditis, characterized by defective formation of the external genitalia (Andersson, S., et al. (1991) Nature 354:159-161; Labrie, F., et al. (1992) Endocrinology 131:1571-1573; OMIM #264600). Thus, OASD plays a central role in sexual differentiation and androgen physiology.

17β-hydroxysteroid dehydrogenase (17βHSD6) plays an important role in the regulation of the male reproductive hormone, dihydrotestosterone (DHTT). 17βHSD6 acts to reduce levels of DHT™ by oxidizing a precursor of DHTT, 3α-diol, to androsterone which is readily glucuronidated and removed from tissues. 17βHSD6 is active with both androgen and estrogen substrates when expressed in embryonic kidney 293 cells. At least five other isozymes of 17βHSD have been identified that catalyze oxidation and/or reduction reactions in various tissues with preferences for different steroid substrates (Biswas, M. G. and Russell, D. W. (1997) J. Biol. Chem. 272:15959-15966). For example, 17βHSD1 preferentially reduces estradiol and is abundant in the ovary and placenta. 17βHSD2 catalyzes oxidation of androgens and is present in the endometrium and placenta. 17βHSD3 is exclusively a reductive enzyme in the testis (Geissler, W. M. et al. (1994) Nature Genet. 7:34-39). An excess of androgens such as DHTT can contribute to certain disease states such as benign prostatic hyperplasia and prostate cancer.

Oxidoreductases are components of the fatty acid metabolism pathways in mitochondria and peroxisomes. The main beta-oxidation pathway degrades both saturated and unsaturated fatty acids, while the auxiliary pathway performs additional steps required for the degradation of unsaturated fatty acids. The auxiliary beta-oxidation enzyme 2,4-dienoyl-CoA reductase catalyzes the removal of even-numbered double bonds from unsaturated fatty acids prior to their entry into the main beta-oxidation pathway. The enzyme may also remove odd-numbered double bonds from unsaturated fatty acids (Koivuranta, K. T. et al. (1994) Biochem. J. 304;787-792; Smeland, T. E. et al. (1992) Proc. Natl. Acad. Sci. USA 89:6673-6677). 2,4-dienoyl-CoA reductase is located in both mitochondria and peroxisomes. Inherited deficiencies in mitochondrial and peroxisomal beta-oxidation enzymes are associated with severe diseases, some of which manifest themselves soon after birth and lead to death within a few years. Defects in beta-oxidation are associated with Reye's syndrome, Zellweger syndrome, neonatal adrenoleukodystrophy, infantile Refsum's disease, acyl-CoA oxidase deficiency, and bifunctional protein deficiency (Suzuki, Y. et al. (1994) Am. J. Hum. Genet. 54:36-43; Hoefler, supra; Cotran, R. S. et al. (1994) Robbins Pathologic Basis of Disease, W. B. Saunders Co., Philadelphia, Pa., p.866). Peroxisomal beta-oxidation is impaired in cancerous tissue. Although neoplastic human breast epithelial cells have the same number of peroxisomes as do normal cells, fatty acyl-CoA oxidase activity is lower than in control tissue (el Bouhtoury, F., et al. (1992) J. Pathol. 166:27-35). Human colon carcinomas have fewer peroxisomes than normal colon tissue and have lower fatty-acyl-CoA oxidase and bifunctional enzyme (including enoyl-CoA hydratase) activities than normal tissue (Cable, S., et al. (1992) Virchows Arch. B Cell Pathol. Incl. Mol. Pathol. 62:221-226). Another important oxidoreductase is isocitrate dehydrogenase, which catalyzes the conversion of isocitrate to a-ketoglutarate, a substrate of the citric acid cycle. Isocitrate dehydrogenase can be either NAD or NADP dependent, and is found in the cytosol, mitochondria, and peroxisomes. Activity of isocitrate dehydrogenase is regulated developmentally, and by hormones, neurotransmitters, and growth factors.

Hydroxypyruvate reductase (HPR), a peroxisomal 2-hydroxyacid dehydrogenase in the glycolate pathway, catalyzes the conversion of hydroxypyruvate to glycerate with the oxidation of both NADH and NADPH. The reverse dehydrogenase reaction reduces NAD⁺ and NADP⁺. HPR recycles nucleotides and bases back into pathways leading to the synthesis of ATP and GTP. ATP and GTP are used to produce DNA and RNA and to control various aspects of signal transduction and energy metabolism. Inhibitors of purine nucleotide biosynthesis have long been employed as antiproliferative agents to treat cancer and viral diseases. HPR also regulates biochemical synthesis of serine and cellular serine levels available for protein synthesis.

The mitochondrial electron transport (or respiratory) chain is a series of oxidoreductase-type enzyme complexes in the mitochondrial membrane that is responsible for the transport of electrons from NADH through a series of redox centers within these complexes to oxygen, and the coupling of this oxidation to the synthesis of ATP (oxidative phosphorylation). ATP then provides the primary source of energy for driving a cell's many energy-requiring reactions. The key complexes in the respiratory chain are NADH:ubiquinone oxidoreductase (complex I), succinate:ubiquinone oxidoreductase (complex II), cytochrome c₁-b oxidoreductase (complex III), cytochrome c oxidase (complex IV), and ATP synthase (complex V) (Alberts, B. et al. (1994) Molecular Biology of the Cell, Garland Publishing, Inc., New York, N.Y., p. 677-678). All of these complexes are located on the inner matrix side of the mitochondrial membrane except complex II, which is on the cytosolic side. Complex II transports electrons generated in the citric acid cycle to the respiratory chain. The electrons generated by oxidation of succinate to fumarate in the citric acid cycle are transferred through electron carriers in complex II to membrane bound ubiquinone (Q). Transcriptional regulation of these nuclear-encoded genes appears to be the predominant means for controlling the biogenesis of respiratory enzymes. Defects and altered expression of enzymes in the respiratory chain are associated with a variety of disease conditions.

Other dehydrogenase activities using NAD as a cofactor are also important in mitochondrial function. 3-hydroxyisobutyrate dehydrogenase (3HBD), important in valine catabolism, catalyzes the NAD-dependent oxidation of 3-hydroxyisobutyrate to methylmalonate semialdehyde within mitochondria.) Elevated levels of 3-hydroxyisobutyrate have been reported in a number of disease states, including ketoacidosis, methylmalonic acidemia, and other disorders associated with deficiencies in methylmalonate semialdehyde dehydrogenase (Rougraff, P. M. et al. (1989) J. Biol. Chem. 264:5899-5903).

Another mitochondrial dehydrogenase important in amino acid metabolism is the enzyme isovaleryl-CoA-dehydrogenase (IVD). IVD is involved in leucine metabolism and catalyzes the oxidation of isovaleryl-CoA to 3-methylcrotonyl-CoA. Human IVD is a tetrameric flavoprotein that is encoded in the nucleus and synthesized in the cytosol as a 45 kDa precursor with a mitochondrial import signal sequence. A genetic deficiency, caused by a mutation in the gene encoding IVD, results in the condition known as isovaleric acidemia. This mutation results in inefficient mitochondrial import and processing of the IVD precursor (Vockley, J. et al. (1992) J. Biol. Chem. 267:2494-2501).

Transferases

Transferases are enzymes that catalyze the transfer of molecular groups. The reaction may involve an oxidation, reduction, or cleavage of covalent bonds, and is often specific to a substrate or to particular sites on a type of substrate. Transferases participate in reactions essential to such functions as synthesis and degradation of cell components, regulation of cell functions including cell signaling, cell proliferation, inflamation, apoptosis, secretion and excretion. Transferases are involved in key steps in disease processes involving these functions. Transferases are frequently classified according to the type of group transferred. For example, methyl transferases transfer one-carbon methyl groups, amino transferases transfer nitrogenous amino groups, and similarly denominated enzymes transfer aldehyde or ketone, acyl, glycosyl, alkyl or aryl, isoprenyl, saccharyl, phosphorous-containing, sulfur-containing, or selenium-containing groups, as well as small enzymatic groups such as Coenzyme A.

Acyl transferases include peroxisomal carnitine octanoyl transferase, which is involved in the fatty acid beta-oxidation pathway, and mitochondrial carnitine palmitoyl transferases, involved in fatty acid metabolism and transport. Choline O-acetyl transferase catalyzes the biosynthesis of the neurotransmitter acetylcholine.

Amino transferases play key roles in protein synthesis and degradation, and they contribute to other processes as well. For example, the amino transferase 5-aminolevulinic acid synthase catalyzes the addition of succinyl-CoA to glycine, the first step in heme biosynthesis. Other amino transferases participate in pathways important for neurological function and metabolism. For example, glutamine-phenylpyruvate amino transferase, also known as glutamine transaminase K (GTK), catalyzes several reactions with a pyridoxal phosphate cofactor. GTK catalyzes the reversible conversion of L-glutamine and phenylpyruvate to 2-oxoglutaramate and L-phenylalanine. Other amino acid substrates for GTK include L-methionine, L-histidine, and L-tyrosine. GTK also catalyzes the conversion of kynurenine to kynurenic acid, a tryptophan metabolite that is an antagonist of the N-methyl-D-aspartate (NMDA) receptor in the brain and may exert a neuromodulatory function. Alteration of the kynurenine metabolic pathway may be associated with several neurological disorders. GTK also plays a role in the metabolism of halogenated xenobiotics conjugated to glutathione, leading to nephrotoxicity in rats and neurotoxicity in humans. GTK is expressed in kidney, liver, and brain. Both human and rat GTKs contain a putative pyridoxal phosphate binding site (ExPASy ENZYME: EC 2.6.1.64; Perry, S. J. et al (1993) Mol. Pharmacol. 43:660-665; Perry, S. et al. (1995) FEBS Lett. 360:277-280; and Alberati-Giani, D. et al. (1995) J. Neurochem. 64:1448-1455). A second amino transferase associated with this pathway is kynurenine/α-aminoadipate amino transferase (AadAT). AadAT catalyzes the reversible conversion of α-aminoadipate and α-ketoglutarate to α-ketoadipate and L-glutamate during lysine metabolism. AadAT also catalyzes the transamination of kynurenine to kynurenic acid. A cytosolic AadAT is expressed in rat kidney, liver, and brain (Nakatani, Y. et al. (1970) Biochim. Biophys. Acta 198:219-228; Buchli, R. et al. (1995) J. Biol. Chem. 270:29330-29335).

Glycosyl transferases include the mammalian UDP-glucouronosyl transferases, a family of membrane-bound microsomal enzymes catalyzing the transfer of glucouronic acid to lipophilic substrates in reactions that play important roles in detoxification and excretion of drugs, carcinogens, and other foreign substances. Another mammalian glycosyl transferase, mammalian UDP-galactose-ceramide galactosyl transferase, catalyzes the transfer of galactose to ceramide in the synthesis of galactocerebrosides in myelin membranes of the nervous system. The UDP-glycosyl transferases share a conserved signature domain of about 50 amino acid residues (PROSITE: PDOC00359, http://expasy.hcuge.ch/sprot/prosite.html).

Methyl transferases are involved in a variety of pharmacologically important processes. Nicotinamide N-methyl transferase catalyzes the N-methylation of nicotinamides and other pyridines, an important step in the cellular handling of drugs and other foreign compounds. Phenylethanolamine N-methyl transferase catalyzes the conversion of noradrenalin to adrenalin. 6-O-methylguanine-DNA methyl transferase reverses DNA methylation, an important step in carcinogenesis. Uroporphyrin-III C-methyl transferase, which catalyzes the transfer of two methyl groups from S-adenosyl-L-methionine to uroporphyrinogen III, is the first specific enzyme in the biosynthesis of cobalamin, a dietary enzyme whose uptake is deficient in pernicious anemia. Protein-arginine methyl transferases catalyze the posttranslational methylation of arginine residues in proteins, resulting in the mono- and dimethylation of arginine on the guanidino group. Substrates include histones, myelin basic protein, and heterogeneous nuclear ribonucleoproteins involved in mRNA processing, splicing, and transport. Protein-arginine methyl transferase interacts with proteins upregulated by mitogens, with proteins involved in chronic lymphocytic leukemia, and with interferon, suggesting an important role for methylation in cytokine receptor signaling (Lin, W.-J. et al. (1996) J. Biol. Chem. 271:15034-15044; Abramovich, C. et al. (1997) EMBO J. 16:260-266; and Scott, H. S. et al. (1998) Genomics 48:330-340).

Phospho transferases catalyze the transfer of high-energy phosphate groups and are important in energy-requiring and -releasing reactions. The metabolic enzyme creatine kinase catalyzes the reversible phosphate transfer between creatine/creatine phosphate and ATP/ADP. Glycocyamine kinase catalyzes phosphate transfer from ATP to guanidoacetate, and arginine kinase catalyzes phosphate transfer from ATP to arginine. A cysteine-containing active site is conserved in this family (PROSITE: PDOC00103).

Prenyl transferases are heterodimers, consisting of an alpha and a beta subunit, that catalyze the transfer of an isoprenyl group. An example of a prenyl transferase is the mammalian protein farnesyl transferase. The alpha subunit of farnesyl transferase consists of 5 repeats of 34 amino acids each, with each repeat containing an invariant tryptophan (PROSITE: PDOC00703).

Saccharyl transferases are glycating enzymes involved in a variety of metabolic processes. Oligosacchryl transferase-48, for example, is a receptor for advanced glycation endproducts. Accumulation of these endproducts is observed in vascular complications of diabetes, macrovascular disease, renal insufficiency, and Alzheimer's disease (Thornalley, P. J. (1998) Cell Mol. Biol. (Noisy-Le-Grand) 44:1013-1023).

Coenzyme A (CoA) transferase catalyzes the transfer of CoA between two carboxylic acids. Succinyl CoA:3-oxoacid CoA transferase, for example, transfers CoA from succinyl-CoA to a recipient such as acetoacetate. Acetoacetate is essential to the metabolism of ketone bodies, which accumulate in tissues affected by metabolic disorders such as diabetes (PROSITE: PDOC00980).

Hydrolases

Hydrolysis is the breaking of a covalent bond in a substrate by introduction of a water molecule. The reaction involves a nucleophilic attack by the water molecule's oxygen atom on a target bond in the substrate. The water molecule is split across the target bond, breaking the bond and generating two product molecules. Hydrolases participate in reactions essential to functions such as cell signaling, cell proliferation, inflammation, apoptosis, secretion and excretion. Hydrolases are involved in key steps in disease processes involving these functions. Hydrolases, or hydrolytic enzymes, may be grouped by substrate specificity into classes including aminohydrolases, phospholipases, carboxyl-esterases, phosphodiesterases, lysozymes, glycosidases, glyoxalases, sulfatases, phosphohydrolases, and serine hydrolases.

Phosphodiesterases catalyze the hydrolysis of one of the two ester bonds in a phosphodiester compound. Phosphodiesterases are, therefore, crucial to a variety of cellular processes. Phosphodiesterases include DNA and RNA endo- and exo-nucleases, which are essential to cell growth and replication as well as protein synthesis.

Pancreatic lipase and colipase form a complex that plays a key role in dietary fat digestion by converting insoluble long chain triacylgycerols into more polar molecules able to cross the brush border of intestinal cells. Colipase binds to the C-terminal domain of lipase.

Carboxylesterases are proteins that hydrolyze carboxylic esters and are classified into three categories—A, B, and C. Most type-B carboxylesterases are evolutionarily related and are considered to comprise a family of proteins. The type-B carboxylesterase family of proteins includes vertebrate acetylcholinesterase, mammalian liver microsomal carboxylesterase, mammalian bile-salt-activated lipase, and duck fatty acyl-CoA hydrolase. Some members of this protein family are not catalytically active but contain a domain related evolutionarily to other type-B carboxylesterases, such as thyroglobulin and Drosphila protein neuractin.

Acyl-CoA thioesterase is another member of the carboxylesterase family (Alexson, S. E., et al. (1993) Eur. J. Biochem. 214(3): 719-727). Evidence suggests that acyl-CoA thioesterase has a regulatory role in steroidogenic tissues (Finkielstein, C., et al. (1998) Eur. J. Biochem. 256(1): 60-66).

A phospholipase A₂ inhibitor has been identified that has 33% sequence homology with human leucine-rich α₂-glycoprotein (Okumura, K., et al. (1998) J. Biol. Chem. 273(31): 19469-19475). Leucine-rich repeat (LRR) consensus sequences have also been found in the primary structure of many proteins, including proteins that participate in biologically important processes, such as receptors for hormones, enzymes, enzyme inhibitors, proteins for cell adhesion, and ribosome-binding proteins. All proteins containing LRR domains are thought to be involved in protein-protein interactions.

Lysozyme c superfamily consists of conventional lysozymes c, calcium-binding lysozymes c, and α-lactalbumin (Prager, E. M. and Jolles, P. (1996) EXS 75: 9-31) The proteins in this superfamily have 35-40% sequence homology and share a common three dimensional fold, but can have different functions. Lysozymes bind and cleave the glycosidic bond linkage in sugars (Iyer, L. K. and Qasba, P. K. (1999) Protein Eng. 12(2): 129-139). Lysozymes c are ubiquitous in a variety of tissues and secretions and can lyse the cell walls of ceratin bacteria (McKenzie, H. A. (1996) EXS 75: 365-409).

The glyoxylase system consists of glyoxalase I, which catalyzes the formation of S-D-lactoylglutathione from methyglyoxal, a side product of triose-phosphate energy metabolism, and glyoxylase II, which hydrolyzes S-D-lactoylglutathione to D-lactic acid and reduced glutathione. Methyglyoxal levels are elevated during hyperglycemia, likely due to increased triose-phosphate energy metabolism. Elevated levels of glyoxylase II activity have been found in human and in a rat model of non-insulin-dependent diabetes mellitus. The glyoxylase system has been implicated in the detoxification of bacterial toxins, and in the control of cell proliferation and microtubule assembly. Elevated levels of S-D-lactoylglutathione, the substrate of glyoxylase II, induced growth arrest and toxicity in HL60 cells. Thus, the glyoxylase system, and glyoxylase II in particular, may be associated with cell proliferation and autoimmune disorders such as diabetes.

The alpha/beta hydrolase fold is a protein fold that is common to several hydrolases of diverse phylogenetic origin and catalytic functions. Enzymes with the alpha/beta hydrolase fold have a common core structure consisting of eight beta-sheets connected by alpha-helices. The best-conserved structural feature of this fold is the loops of the nucleophile-histidine-acid catalytic triad. The histidine in the catalytic triad is completely conserved, while the nucleophile and acid loops accommodate more than one type of amino acid (Ollis, D. L., et al. (1992) Protein Eng. 5:197-211).

Sulfatases are members of a highly conserved gene family that share extensive sequence homology and a high degree of structural similarity. Sulfatases catalyze the cleavage of sulfate esters. To perform this function, sulfatases undergo a unique posttranslational modification in the endoplasmic reticulum that involves the oxidation of a conserved cysteine residue. A human disorder called multiple sulfatase deficiency is due to a defect in this posttranslational modification step, leading to inactive sulfatases (Recksiek, M., et al. (1998) J. Biol. Chem. 273(11): 6096-6103).

Phosphohydrolases are enzymes that hydrolyze phosphate esters. Some phosphohydrolases contain a mutT domain signature sequence. MutT is a protein involved in the GO system responsible for removing an oxidatively damaged form of guanine from DNA.

Glycosidases catalyze the cleavage of hemiacetyl bonds of glycosides, which are compounds that contain one or more sugar. Mammalian beta-galactosidase removes the terminal galactose from gangliosides, glycoproteins, and glycosaminoglycans. Beta-galactosidases belong to family 35 in the classification of glycosyl hydrolases.

Serine hydrolases are a functional class of hydrolytic enzymes that contain a serine residue in their active site. This class of enzymes contains proteinases, esterases, and lipases which hydrolyze a variety of substrates and, therefore, have different biological roles. Proteins in this superfamily can be further grouped into subfamilies based on substrate specificity or amino acid similarities (Puente, X. S. and Lopez-Ont, C. (1995) J. Biol. Chem. 270(21): 12926-12932).

Lyases

Lyases are a class of enzymes that catalyze the cleavage of C—C, C—O, C—N, C—S, C-(halide), P—O or other bonds without hydrolysis or oxidation to form two molecules, at least one of which contains a double bond (Stryer, L. (1995) Biochemistry W. H. Freeman and Co. New York, N.Y. p.620). Lyases are critical components of cellular biochemistry with roles in metabolic energy production including fatty acid metabolism, as well as other diverse enzymatic processes. Further classification of lyases reflects the type of bond cleaved as well as the nature of the cleaved group.

The group of C—C lyases include carboxyl-lyases (decarboxylases), aldehyde-lyases (aldolases), oxo-acid-lyases and others. The C—O lyase group includes hydro-lyases, lyases acting on polysaccharides and other lyases. The C—N lyase group includes ammonia-lyases, amidine-lyases, amine-lyases (deaminases) and other lyases.

Proper regulation of lyases is critical to normal physiology. For example, mutation induced deficiencies in the uroporphyrinogen decarboxylase can lead to photosensitive cutaneous lesions in the genetically-linked disorder familial porphyria cutanea tarda (Mendez, M. et al. (1998) Am. J. Genet. 63:1363-1375). It has also been shown that adenosine deaminase (ADA) deficiency stems from genetic mutations in the ADA gene, resulting in the disorder severe combined immunodeficiency disease (SCID) (Hershfield, M. S. (1998) Semin. Hematol. 35:291-298).

Isomerases

Isomerases are a class of enzymes that catalyze geometric or structural changes within a molecule to form a single product. This class includes racemases and epimerases, cis-trans-isomerases, intramolecular oxidoreductases, intramolecular transferases (mutases) and intramolecular lyases. Isomerases are critical components of cellular biochemistry with roles in protein folding, phototransduction, and metabolic energy production including glycolysis, as well as other diverse enzymatic processes (Stryer, L. (1995) Biochemistry W. H. Freeman and Co. New York, N.Y. pp.483-507).

Racemases are a subset of isomerases that catalyze inversion of a molecules configuration around the asymmetric carbon atom in a substrate having a single center of asymmetry, thereby interconverting two racemers. Epimerases are another subset of isomerases that catalyze inversion of configuration around an asymmetric carbon atom in a substrate with more than one center of symmetry, thereby interconverting two epimers. Racemases and epimerases can act on amino acids and derivatives, hydroxy acids and derivatives, as well as carbohydrates and derivatives. The interconversion of UDP-galactose and UDP-glucose is catalyzed by UDP-galactose-4′-epimerase. Proper regulation and function of this epimerase is essential to the synthesis of glycoproteins and glycolipids. Elevated blood galactose levels have been correlated with UDP-galactose-4′-epimerase deficiency in screening programs of infants (Gitzelmann, R. (1972) Helv. Paediat. Acta 27:125-130).

The peptidyl prolyl cis-trans isomerases (PPIases) are a class of folding enzymes that isomerize certain proline imidic bonds in what is considered to be a rate limiting step in protein maturation and export. PPIases catalyze the cis to trans isomerization of certain proline imidic bonds in proteins. There are three evolutionarily unrelated families of PPIases: the cyclophilins, the FK506 binding proteins, and the newly characterized parvulin family (Rahfeld, J. U. et al. (1994) FEBS Lett. 352: 180-184).

The cyclophilins (CyP) were originally identified as major receptors for the immunosuppressive drug cyclosporin A (CsA), an inhibitor of T-cell activation (Handschumacher, R. E. et al. (1984) Science 226: 544-547; Harding, M. W. et al. (1986) J. Biol. Chem. 261: 8547-8555). Thus, the peptidyl-prolyl isomerase activity of CyP may be part of the signaling pathway that leads to T-cell activation. Subsequent work demonstrated that CyP's isomerase activity is essential for correct protein folding and/or protein trafficking, and may also be involved in assembly/disassembly of protein complexes and regulation of protein activity. For example, in Drosophila, the CyP NinaA is required for correct localization of rhodopsins, while a mammalian CyP (Cyp40) is part of the Hsp90/Hsp70 complex that binds steroid receptors. The mammalian CyP (CypA) has been shown to bind the gag protein from human immunodeficiency virus 1 (HIV-1), an interaction that can be inhibited by cyclosporin. Since cyclosporin has potent anti-HIV-1 activity, CypA may play an essential function in HIV-1 replication. Finally, Cyp4O has been shown to bind and inactivate the transcription factor c-Myb, an effect that is reversed by cyclosporin. This effect implicates CyP in the regulation of transcription, transformation, and differentiation (Bergsma, D. J. et al (1991) J. Biol. Chem. 266:23204-23214; Hunter, T. (1998) Cell 92: 141-143; and Leverson, J. D. and Ness, S. A. (1998) Mol. Cell. 1:203-211).

Another class of folding enzymes are the protein disulfide isomerases. One of the major rate limiting steps in protein folding is the thiol:disulfide exchange that is necessary for correct protein assembly. Although incubation of reduced, unfolded proteins in buffers with defined ratios of oxidized and reduced thiols can lead to native conformation, the rate of folding is slow and the attainment of native conformation decreases proportionately with the size and number of cysteines in the protein. Certain cellular compartments such as the endoplasmic reticulum of eukaryotes and the periplasmic space of prokaryotes are maintained in a more oxidized state than the surrounding cytosol. Correct disulfide formation can occur in these compartments, but at a rate that is insufficient for normal cell processes and inadequate for synthesizing secreted proteins. The protein disulfide isomerases, thioredoxins and glutaredoxins are able to catalyze the formation of disulfide bonds and regulate the redox environment in cells to enable the necessary thiol:disulfide exchanges (Loferer, H. (1995) J. Biol. Chem. 270:26178-26183).

Each of these proteins has somewhat different functions, but all belong to a group of disulfide-containing redox proteins that contain a conserved active-site sequence and are ubiquitously distributed in eukaryotes and prokaryotes. Protein disulfide isomerases are found in the endoplasmic reticulum of eukaryotes and in the periplasmic space of prokaryotes. They function by exchanging their own disulfide for a thiol in a folding peptide chain. In contrast, the reduced thioredoxins and glutaredoxins are generally found in the cytoplasm and function by directly reducing disulfides in the substrate proteins.

These catalytic molecules not only facilitate disulfide formation, but also regulate and participate in a wide variety of physiological processes. The thioredoxin system serves, for example, as a hydrogen donor for ribonucleotide reductase and as a regulator of enzymes by redox control. It also modulates the activity of transcription factors such as NF-κB, AP-1, and steroid receptors. More recently, several cytokines or secreted cytokine-like factors such as adult T-cell leukemia-derived factor, 3B6-interleukin-1, T-hybridoma-derived (MP-6) B cell stimulatory factor, and early pregnancy factor have been reported to be identical to thioredoxin (Holmgren, A. (1985) Annu. Rev. Biochem. 54:237-271; Abate, C. et al., (1990) Science 249:1157-1161; Tagaya, Y. et al. (1989) EMBO J. 8:757-764;, Wakasugi, H. (1987) Proc. Natl. Acad. Sci. USA 84:804-808; Rosen, A. et al. (1995) Int. Immunol. 7:625-633). Thioredoxin has also been shown to have many extracellular activities including a role as a regulator of cell growth and a mediator in the immune system (Miranda-Vizuete, A. et al. (1996) J. Biol. Chem. 271:19099-19103; Yamauchi, A. et al (1992) Mol. Immunol. 29:263-270).

Oxidoreductases can be isomerases as well. Oxidoreductases catalyze the reversible transfer of electrons from a substrate that becomes oxidized to a substrate that becomes reduced. This class of enzymes includes dehydrogenases, hydroxylases, oxidases, oxygenases, peroxidases, and reductases. Proper maintenance of oxidoreductase levels is physiologically important. The pentose phosphate pathway for example, utilizes enzymes which are responsible for generating the reducing agent NADPH, while at the same time oxidizing glucose-6-phosphate to ribose-5-phosphate. NADPH serves as the fuel for reactions undergoing reductive biosynthesis. Ribose-5-phosphate and its derivatives become part of critical biological molecules such as ATP, Coenzyme A, NAD⁺, FAD, RNA, and DNA. The pentose phosphate pathway has both oxidative and non-oxidative branches. The oxidative branch steps, which are catalyzed by the enzymes glucose-6-phosphate dehydrogenase, lactonase, and 6-phosphogluconate dehydrogenase, convert glucose-6-phosphate and NADP⁺ to ribulose-6-phosphate and NADPH. The non-oxidative branch steps, which are catalyzed by the enzymes phosphopentose isomerase, phosphopentose epimerase, transketolase, and transaldolase, allow the interconversion of three-, four-, five-, six-, and seven-carbon sugars.

Another subgroup of isomerases are the transferases (or mutases). Transferases transfer a chemical group from one compound (the donor) to another compound (the acceptor). The types of groups transferred by these enzymes include acyl groups, amino groups, phosphate groups (phosphotransferases or phosphomutases), and others. The transferase carnitine palmitoyltransferase is an important component of fatty acid metabolism. Genetically-linked deficiencies in this transferase can lead to myopathy (Scriver C. R. et. al. (1995) The Metabolic and Molecular Basis of Inherited Disease, McGraw-Hill New York N.Y. pp. 1501-1533).

Yet another subgroup of isomerases are the topoisomersases. Topoisomerases are enzymes that affect the topological state of DNA. For example, defects in topoisomerases or their regulation can affect normal physiology. Reduced levels of topoisomerase II have been correlated with some of the DNA processing defects associated with the disorder ataxia-telangiectasia (Singh, S. P. et. al. (1988) Nucleic Acids Res. 16:3919-3929).

Ligases

Ligases catalyze the formation of a bond between two substrate molecules. The process involves the hydrolysis of a pyrophosphate bond in ATP or a similar energy donor. Ligases are classified based on the nature of the type of bond they form, which can include carbon-oxygen, carbon-sulfur, carbon-nitrogen, carbon-carbon and phosphoric ester bonds.

Ligases forming carbon-oxygen bonds include the aminoacyl-transfer RNA (tRNA) synthetases which are important RNA-associated enzymes with roles in translation. Protein biosynthesis depends on each amino acid forming a linkage with the appropriate tRNA. The aminoacyl-tRNA synthetases are responsible for the activation and correct attachment of an amino acid with its cognate tRNA. The 20 aminoacyl-tRNA synthetase enzymes can be divided into two structural classes, and each class is characterized by a distinctive topology of the catalytic domain. Class I enzymes contain a catalytic domain based on the nucleotide-binding Rossman ‘fold’. Class II enzymes contain a central catalytic domain, which consists of a seven-stranded antiparallel 1-sheet motif, as well as N- and C-terminal regulatory domains. Class II enzymes are separated into two groups based on the heterodimeric or homodimeric structure of the enzyme; the latter group is further subdivided by the structure of the N- and C-terminal regulatory domains (Hartlein, M. and Cusack, S. (1995) J. Mol. Evol. 40:519-530). Autoantibodies against aminoacyl-tRNAs are generated by patients with dermatomyositis and polymyositis, and correlate strongly with complicating interstitial lung disease (ILD). These antibodies appear to be generated in response to viral infection, and coxsackie virus has been used to induce experimental viral myositis in animals.

Ligases forming carbon-sulfur bonds (Acid-thiol ligases) mediate a large number of cellular biosynthetic intermediary metabolism processes involve intermolecular transfer of carbon atom-containing substrates (carbon substrates). Examples of such reactions include the tricarboxylic acid cycle, synthesis of fatty acids and long-chain phospholipids, synthesis of alcohols and aldehydes, synthesis of intermediary metabolites, and reactions involved in the amino acid degradation pathways. Some of these reactions require input of energy, usually in the form of conversion of ATP to either ADP or AMP and pyrophosphate.

In many cases, a carbon substrate is derived from a small molecule containing at least two carbon atoms. The carbon substrate is often covalently bound to a larger molecule which acts as a carbon substrate carrier molecule within the cell. In the biosynthetic mechanisms described above, the carrier molecule is coenzyme A. Coenzyme A (CoA) is structurally related to derivatives of the nucleotide ADP and consists of 4′-phosphopantetheine linked via a phosphodiester bond to the alpha phosphate group of adenosine 3′,5′-bisphosphate. The terminal thiol group of 4′-phosphopantetheine acts as the site for carbon substrate bond formation. The predominant carbon substrates which utilize CoA as a carrier molecule during biosynthesis and intermediary metabolism in the cell are acetyl, succinyl, and propionyl moieties, collectively referred to as acyl groups. Other carbon substrates include enoyl lipid, which acts as a fatty acid oxidation intermediate, and carnitine, which acts as an acetyl-CoA flux regulator/mitochondrial acyl group transfer protein. Acyl-CoA and acetyl-CoA are synthesized in the cell by acyl-CoA synthetase and acetyl-CoA synthetase, respectively.

Activation of fatty acids is mediated by at least three forms of acyl-CoA synthetase activity: i) acetyl-CoA synthetase, which activates acetate and several other low molecular weight carboxylic acids and is found in muscle mitochondria and the cytosol of other tissues; ii) medium-chain acyl-CoA synthetase, which activates fatty acids containing between four and eleven carbon atoms (predominantly from dietary sources), and is present only in liver mitochondria; and iii) acyl CoA synthetase, which is specific for long chain fatty acids with between six and twenty carbon atoms, and is found in microsomes and the mitochondria. Proteins associated with acyl-CoA synthetase activity have been identified from many sources including bacteria, yeast, plants, mouse, and man. The activity of acyl-CoA synthetase may be modulated by phosphorylation of the enzyme by cAMP-dependent protein kinase.

Ligases forming carbon-nitrogen bonds include amide synthases such as glutamine synthetase (glutamate-ammonia ligase) that catalyzes the amination of glutamic acid to glutamine by ammonia using the energy of ATP hydrolysis. Glutamine is the primary source for the amino group in various amide transfer reactions involved in de novo pyrimidine nucleotide synthesis and in purine and pyrimidine ribonucleotide interconversions. Overexpression of glutamine synthetase has been observed in primary liver cancer (Christa, L. et al. (1994) Gastroent. 106:1312-1320).

Acid-amino-acid ligases (peptide synthases) are represented by the ubiquitin proteases which are associated with the ubiquitin conjugation system (UCS), a major pathway for the degradation of cellular proteins in eukaryotic cells and some bacteria. The UCS mediates the elimination of abnormal proteins and regulates the half-lives of important regulatory proteins that control cellular processes such as gene transcription and cell cycle progression. In the UCS pathway, proteins targeted for degradation are conjugated to a ubiquitin (Ub), a small heat stable protein. Ub is first activated by a ubiquitin-activating enzyme (E1), and then transferred to one of several Ub-conjugating enzymes (E2). E2 then links the Ub molecule through its C-terminal glycine to an internal lysine (acceptor lysine) of a target protein. The ubiquitinated protein is then recognized and degraded by proteasome, a large, multisubunit proteolytic enzyme complex, and ubiquitin is released for reutilization by ubiquitin protease. The UCS is implicated in the degradation of mitotic cyclic kinases, oncoproteins, tumor suppressor genes such as p53, viral proteins, cell surface receptors associated with signal transduction, transcriptional regulators, and mutated or damaged proteins (Ciechanover, A. (1994) Cell 79:13-21). A murine proto-oncogene, Unp, encodes a nuclear ubiquitin protease whose overexpression leads to oncogenic transformation of NIH3T3 cells, and the human homolog of this gene is consistently elevated in small cell tumors and adenocarcinomas of the lung (Gray, D. A. (1995) Oncogene 10:2179-2183).

Cyclo-ligases and other carbon-nitrogen ligases comprise various enzymes and enzyme complexes that participate in the de novo pathways to purine and pyrimidine biosynthesis. Because these pathways are critical to the synthesis of nucleotides for replication of both RNA and DNA, many of these enzymes have been the targets of clinical agents for the treatment of cell proliferative disorders such as cancer and infectious diseases.

Purine biosynthesis occurs de novo from the amino acids glycine and glutamine, and other small molecules. Three of the key reactions in this process are catalyzed by a trifunctional enzyme composed of glycinamide-ribonucleotide synthetase (GARS), aminoimidazole ribonucleotide synthetase (AIRS), and glycinamide ribonucleotide transformylase (GART). Together these three enzymes combine ribosylamine phosphate with glycine to yield phosphoribosyl aminoimidazole, a precursor to both adenylate and guanylate nucleotides. This trifunctional protein has been implicated in the pathology of Downs syndrome (Aimi, J. et al. (1990) Nucleic Acid Res. 18:6665-6672). Adenylosuccinate synthetase catalyzes a later step in purine biosynthesis that converts inosinic acid to adenylosuccinate, a key step on the path to ATP synthesis. This enzyme is also similar to another carbon-nitrogen ligase, argininosuccinate synthetase, that catalyzes a similar reaction in the urea cycle (Powell, S. M. et al. (1992) FEBS Lett. 303:4-10).

Like the de novo biosynthesis of purines, de novo synthesis of the pyrimidine nucleotides uridylate and cytidylate also arises from a common precursor, in this instance the nucleotide orotidylate derived from orotate and phosphoribosyl pyrophosphate (PPRP). Again a trifunctional enzyme comprising three carbon-nitrogen ligases plays a key role in the process. In this case the enzymes aspartate transcarbamylase (ATCase), carbamyl phosphate synthetase II, and dihydroorotase (DHOase) are encoded by a single gene called CAD. Together these three enzymes combine the initial reactants in pyrimidine biosynthesis, glutamine, CO₂, and ATP to form dihydroorotate, the precursor to orotate and orotidylate (Iwahana, H. et al. (1996) Biochem. Biophys. Res. Commun. 219:249-255). Further steps then lead to the synthesis of uridine nucleotides from orotidylate. Cytidine nucleotides are derived from uridine-5′-triphosphate (UTP) by the amidation of UTP using glutamine as the amino donor and the enzyme CTP synthetase. Regulatory mutations in the human CTP synthetase are believed to confer multi-drug resistance to agents widely used in cancer therapy (Yamauchi, M. et al. (1990) EMBO J. 9:2095-2099).

Ligases forming carbon-carbon bonds include the carboxylases acetyl-CoA carboxylase and pyruvate carboxylase. Acetyl-CoA carboxylase catalyzes the carboxylation of Acetyl-CoA from CO 2 and H₂O using the energy of ATP hydrolysis. Acetyl-CoA carboxylase is the rate-limiting step in the biogenesis of long-chain fatty acids. Two isoforms of Acetyl-CoA carboxylase, types I and types II, are expressed in human in a tissue-specific manner (Ha, J. et al. (1994) Eur. J. Biochem. 219:297-306). Pyruvate carboxylase is a nuclear-encoded mitochondrial enzyme that catalyzes the conversion of pyruvate to oxaloacetate, a key intermediate in the citric acid cycle.

Ligases forming phosphoric ester bonds include the DNA ligases involved in both DNA replication and repair. DNA ligases seal phosphodiester bonds between two adjacent nucleotides in a DNA chain using the energy from ATP hydrolysis to first activate the free 5′-phosphate of one nucleotide and then react it with the 3′-OH group of the adjacent nucleotide. This resealing reaction is used in both DNA replication to join small DNA fragments called “Okazaki” fragments that are transiently formed in the process of replicating new DNA, and in DNA repair. DNA repair is the process by which accidental base changes, such as those produced by oxidative damage, hydrolytic attack, or uncontrolled methylation of DNA, are corrected before replication or transcription of the DNA can occur. Bloom's syndrome is an inherited human disease in which individuals are partially deficient in DNA ligation and consequently have an increased incidence of cancer (Alberts, B. et al. (1994) The Molecular Biology of the Cell, Garland Publishing Inc., New York, N.Y., p. 247).

Cofactor Biosynthetic Enzymes

Cofactors, including coenzymes and prosthetic groups, are small molecular weight inorganic or organic compounds that are required for the action of an enzyme. One particular cofactor, the molybdenum-containing cofactor molybdopterin, is an ubiquitous molecule which is required for the activity of a variety of enzymes, including nitrate reductase, sulfite oxidase, xanthine dehydrogenase, and aldehyde oxidase.

Molybdopterin biosynthesis is performed via a two step reaction pathway. First, a guanosine derivative is converted to an intermediate called precursor Z. Precursor Z is then converted to molybdopterin. In humans, the MOCS1 transcript encodes two enzymes, MOCS1 A and MOCS1 B, which are involved in the first step. MOCS2 is also a single transcript which encodes the two subunits of molybdopterin synthase, the enzyme catalyzing the second step, in two overlapping reading frames (Reiss, J. et al. (1999) Am. J. Hum. Genet. 64:706-11). In Aspergillus nidulans, CnxABC catalyzes the first step in the molybdopterin biosynthesis pathway. The second step in A. nidulans is catalyzed by molybdopterin synthase, as it is in humans. In addition, a converting factor, CnxF, has also been discovered in A. nidulans (Appleyard, M. et al., (1998) J. Biol. Chem. 273:14869-14876). CnxF is similar to E. coli MoeB, an enzyme which transfers sulfur atoms to the synthase and makes it capable of adding the dithiolene group to precursor Z. CnxF is thought to mediate the same reaction in A. nidulans. CnxF is also similar to ThiF, an enzyme required for thiamin biosynthesis; HesA, which is involved in hetercyst formation; and the eukaryotic ubiquitin-activating protein El. However, no obvious physiological relationship exists between CnxF and these latter proteins.

A deficiency in molybdopterin biosynthesis will result in the loss of molybdopterin-dependent enzyme activity. Deficiencies in molybdopterin-dependent enzymes cause neonatal seizures, mental retardation and lens dislocation. Other diseases caused by defects in cofactor metabolism include pernicious anemia and methylmalonic aciduria.

The discovery of new human enzyme molecules and the polynucleotides encoding them satisfies a need in the art by providing new compositions which are useful in the diagnosis, prevention, and treatment of autoimmune/inflammation disorders, genetic disorders, neurological disorders, and cell proliferative disorders including cancer, and in the assessment of the effects of exogenous compounds on the expression of nucleic acid and amino acid sequences of human enzyme molecules.

### SUMMARY OF THE INVENTION

The invention features purified polypeptides, human enzyme molecules, referred to collectively as “HEM” and individually as “HEM-1,” “HEM-2,” “HEM-3,” “HEM-4,” “HEM-14,” “HEM-15,” “HEM-16,” “HEM-17,” “HEM-18,” “HEM-19,” “HEM-20,” “HEM-21,” “HEM-22,” “HEM-23,” “HEM-24,” “HEM-25,” and “HEM-26.” In one aspect, the invention provides an isolated polypeptide comprising an amino acid sequence selected from the group consisting of a) an amino acid sequence selected from the group consisting of SEQ ID NO:1-26, b) a naturally occurring amino acid sequence having at least 90% sequence identity to an amino acid sequence selected from the group consisting of SEQ ID NO:1-26, c) a biologically active fragment of an amino acid sequence selected from the group consisting of SEQ ID NO:1-26, and d) an immunogenic fragment of an amino acid sequence selected from the group consisting of SEQ ID NO:1-26. In one alternative, the invention provides an isolated polypeptide comprising the amino acid sequence of SEQ ID NO:1-26.

The invention further provides an isolated polynucleotide encoding a polypeptide comprising an amino acid sequence selected from the group consisting of a) an amino acid sequence selected from the group consisting of SEQ ID NO:1-26, b) a naturally occurring amino acid sequence having at least 90% sequence identity to an amino acid sequence selected from the group consisting of SEQ ID NO:1-26, c) a biologically active fragment of an amino acid sequence selected from the group consisting of SEQ ID NO:1-26, and d) an immunogenic fragment of an amino acid sequence selected from the group consisting of SEQ ID NO:1-26. In one alternative, the polynucleotide encodes a polypeptide selected from the group consisting of SEQ ID NO:1-26. In another alternative, the polynucleotide is selected from the group consisting of SEQ ID NO:27-52.

Additionally, the invention provides a recombinant polynucleotide comprising a promoter sequence operably linked to a polynucleotide encoding a polypeptide comprising an amino acid sequence selected from the group consisting of a) an amino acid sequence selected from the group consisting of SEQ ID NO:1-26, b) a naturally occurring amino acid sequence having at least 90% sequence identity to an amino acid sequence selected from the group consisting of SEQ ID NO:1-26, c) a biologically active fragment of an amino acid sequence selected from the group consisting of SEQ ID NO:1-26, and d) an immunogenic fragment of an amino acid sequence selected from the group consisting of SEQ ID NO:1-26. In one alternative, the invention provides a cell transformed with the recombinant polynucleotide. In another alternative, the invention provides a transgenic organism comprising the recombinant polynucleotide.

The invention also provides a method for producing a polypeptide comprising an amino acid sequence selected from the group consisting of a) an amino acid sequence selected from the group consisting of SEQ ID NO:1-26, b) a naturally occurring amino acid sequence having at least 90% sequence identity to an amino acid sequence selected from the group consisting of SEQ ID NO:1-26, c) a biologically active fragment of an amino acid sequence selected from the group consisting of SEQ ID NO:1-26, and d) an immunogenic fragment of an amino acid sequence selected from the group consisting of SEQ ID NO:1-26. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Additionally, the invention provides an isolated antibody which specifically binds to a polypeptide comprising an amino acid sequence selected from the group consisting of a) an amino acid sequence selected from the group consisting of SEQ ID NO:1-26, b) a naturally occurring amino acid sequence having at least 90% sequence identity to an amino acid sequence selected from the group consisting of SEQ ID NO:1-26, c) a biologically active fragment of an amino acid sequence selected from the group consisting of SEQ ID NO:1-26, and d) an immunogenic fragment of an amino acid sequence selected from the group consisting of SEQ ID NO:1-26.

The invention further provides an isolated polynucleotide comprising a polynucleotide sequence selected from the group consisting of a) a polynucleotide sequence selected from the group consisting of SEQ ID NO:27-52, b) a naturally occurring polynucleotide sequence having at least 90% sequence identity to a polynucleotide sequence selected from the group consisting of SEQ ID NO:27-52, c) a polynucleotide sequence complementary to a), d) a polynucleotide sequence complementary to b), and e) an RNA equivalent of a)-d). In one alternative, the polynucleotide comprises at least 60 contiguous nucleotides.

Additionally, the invention provides a method for detecting a target polynucleotide in a sample, said target polynucleotide having a sequence of a polynucleotide comprising a polynucleotide sequence selected from the group consisting of a) a polynucleotide sequence selected from the group consisting of SEQ ID NO:27-52, b) a naturally occurring polynucleotide sequence having at least 90% sequence identity to a polynucleotide sequence selected from the group consisting of SEQ ID NO:27-52, c) a polynucleotide sequence complementary to a), d) a polynucleotide sequence complementary to b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex, and optionally, if present, the amount thereof. In one alternative, the probe comprises at least 60 contiguous nucleotides.

The invention further provides a method for detecting a target polynucleotide in a sample, said target polynucleotide having a sequence of a polynucleotide comprising a polynucleotide sequence selected from the group consisting of a) a polynucleotide sequence selected from the group consisting of SEQ ID NO:27-52, b) a naturally occurring polynucleotide sequence having at least 90% sequence identity to a polynucleotide sequence selected from the group consisting of SEQ ID NO:27-52, c) a polynucleotide sequence complementary to a), d) a polynucleotide sequence complementary to b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof, and, optionally, if present, the amount thereof.

The invention further provides a composition comprising an effective amount of a polypeptide comprising an amino acid sequence selected from the group consisting of a) an amino acid sequence selected from the group consisting of SEQ ID NO:1-26, b) a naturally occurring amino acid sequence having at least 90% sequence identity to an amino acid sequence selected from the group consisting of SEQ ID NO:1-26, c) a biologically active fragment of an amino acid sequence selected from the group consisting of SEQ ID NO:1-26, and d) an immunogenic fragment of an amino acid sequence selected from the group consisting of SEQ ID NO:1-26, and a pharmaceutically acceptable excipient. In one embodiment, the composition comprises an amino acid sequence selected from the group consisting of SEQ ID NO:1-26. The invention additionally provides a method of treating a disease or condition associated with decreased expression of functional HEM, comprising administering to a patient in need of such treatment the composition.

The invention also provides a method for screening a compound for effectiveness as an agonist of a polypeptide comprising an amino acid sequence selected from the group consisting of a) an amino acid sequence selected from the group consisting of SEQ ID NO:1-26, b) a naturally occurring amino acid sequence having at least 90% sequence identity to an amino acid sequence selected from the group consisting of SEQ ID NO:1-26, c) a biologically active fragment of an amino acid sequence selected from the group consisting of SEQ ID NO:1-26, and d) an immunogenic fragment of an amino acid sequence selected from the group consisting of SEQ ID NO:1-26. The method comprises a) exposing a sample comprising the polypeptide to a compound, and b) detecting agonist activity in the sample. In one alternative, the invention provides a composition comprising an agonist compound identified by the method and a pharmaceutically acceptable excipient. In another alternative, the invention provides a method of treating a disease or condition associated with decreased expression of functional HEM, comprising administering to a patient in need of such treatment the composition.

Additionally, the invention provides a method for screening a compound for effectiveness as an antagonist of a polypeptide comprising an amino acid sequence selected from the group consisting of a) an amino acid sequence selected from the group consisting of SEQ ID NO:1-26, b) a naturally occurring amino acid sequence having at least 90% sequence identity to an amino acid sequence selected from the group consisting of SEQ ID NO:1-26, c) a biologically active fragment of an amino acid sequence selected from the group consisting of SEQ ID NO:1-26, and d) an immunogenic fragment of an amino acid sequence selected from the group consisting of SEQ ID NO:1-26. The method comprises a) exposing a sample comprising the polypeptide to a compound, and b) detecting antagonist activity in the sample. In one alternative, the invention provides a composition comprising an antagonist compound identified by the method and a pharmaceutically acceptable excipient. In another alternative, the invention provides a method of treating a disease or condition associated with overexpression of functional HEM, comprising administering to a patient in need of such treatment the composition.

The invention further provides a method of screening for a compound that specifically binds to a polypeptide comprising an amino acid sequence selected from the group consisting of a) an amino acid sequence selected from the group consisting of SEQ ID NO:1-26, b) a naturally occurring amino acid sequence having at least 90% sequence identity to an amino acid sequence selected from the group consisting of SEQ ID NO:1-26, c) a biologically active fragment of an amino acid sequence selected from the group consisting of SEQ ID NO:1-26, and d) an immunogenic fragment of an amino acid sequence selected from the group consisting of SEQ ID NO:1-26. The method comprises a) combining the polypeptide with at least one test compound under suitable conditions, and b) detecting binding of the polypeptide to the test compound, thereby identifying a compound that specifically binds to the polypeptide.

The invention further provides a method of screening for a compound that modulates the activity of a polypeptide comprising an amino acid sequence selected from the group consisting of a) an amino acid sequence selected from the group consisting of SEQ ID NO:1-26, b) a naturally occurring amino acid sequence having at least 90% sequence identity to an amino acid sequence selected from the group consisting of SEQ ID NO:1-26, c) a biologically active fragment of an amino acid sequence selected from the group consisting of SEQ ID NO:1-26, and d) an immunogenic fragment of an amino acid sequence selected from the group consisting of SEQ ID NO:1-26. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

The invention further provides a method for screening a compound for effectiveness in altering expression of a target polynucleotide, wherein said target polynucleotide comprises a sequence selected from the group consisting of SEQ ID NO:27-52, the method comprising a) exposing a sample comprising the target polynucleotide to a compound, and b) detecting altered expression of the target polynucleotide.

The invention further provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide comprising a polynucleotide sequence selected from the group consisting of i) a polynucleotide sequence selected from the group consisting of SEQ ID NO:27-52, ii) a naturally occurring polynucleotide sequence having at least 90% sequence identity to a polynucleotide sequence selected from the group consisting of SEQ ID NO:27-52, iii) a polynucleotide sequence complementary to i), iv) a polynucleotide sequence complementary to ii), and v) an RNA equivalent of i)-iv). Hybridization occurs under conditions whereby a specific hybridization complex is formed between said probe and a target polynucleotide in the biological sample, said target polynucleotide comprising a polynucleotide sequence selected from the group consisting of i) a polynucleotide sequence selected from the group consisting of SEQ ID NO:27-52, ii) a naturally occurring polynucleotide sequence having at least 90% sequence identity to a polynucleotide sequence selected from the group consisting of SEQ ID NO:27-52, iii) a polynucleotide sequence complementary to i), iv) a polynucleotide sequence complementary to ii), and v) an RNA equivalent of i)-iv). Alternatively, the target polynucleotide comprises a fragment of a polynucleotide sequence selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for the full length polynucleotide and polypeptide sequences of the present invention.

Table 2 shows the GenBank identification number and annotation of the nearest GenBank homolog for polypeptides of the invention. The probability score for the match between each polypeptide and its GenBank homolog is also shown.

Table 3 shows structural features of polypeptide sequences of the invention, including predicted motifs and domains, along with the methods, algorithms, and searchable databases used for analysis of the polypeptides.

Table 4 lists the cDNA fragments which were used to assemble polynucleotide sequences of the invention, along with selected fragments of the polynucleotide sequences.

Table 5 shows the representative cDNA library for polynucleotides of the invention.

Table 6 provides an appendix which describes the tissues and vectors used for construction of the cDNA libraries shown in Table 5.

Table 7 shows the tools, programs, and algorithms used to analyze the polynucleotides and polypeptides of the invention, along with applicable descriptions, references, and threshold parameters.

### DESCRIPTION OF THE INVENTION

Before the present proteins, nucleotide sequences, and methods are described, it is understood that this invention is not limited to the particular machines, materials and methods described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

Definitions

“HEM” refers to the amino acid sequences of substantially purified HEM obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of HEM. Agonists may include proteins, nucleic acids, carbohydrates, small molecules, or any other compound or composition which modulates the activity of HEM either by directly interacting with HEM or by acting on components of the biological pathway in which HEM participates.

An “allelic variant” is an alternative form of the gene encoding HEM. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding HEM include those sequences with deletions, insertions, or substitutions of different nucleotides, resulting in a polypeptide the same as HEM or a polypeptide with at least one functional characteristic of HEM. Included within this definition are polymorphisms which may or may not be readily detectable using a particular oligonucleotide probe of the polynucleotide encoding HEM, and improper or unexpected hybridization to allelic variants, with a locus other than the normal chromosomal locus for the polynucleotide sequence encoding HEM. The encoded protein may also be “altered,” and may contain deletions, insertions, or substitutions of amino acid residues which produce a silent change and result in a functionally equivalent HEM. Deliberate amino acid substitutions may be made on the basis of similarity in polarity, charge, solubility, hydrophobicity, hydrophilicity, and/or the amphipathic nature of the residues, as long as the biological or immunological activity of HEM is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” refer to an oligopeptide, peptide, polypeptide, or protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid sequence. Amplification is generally carried out using polymerase chain reaction (PCR) technologies well known in the art.

The term “antagonist” refers to a molecule which inhibits or attenuates the biological activity of HEM. Antagonists may include proteins such as antibodies, nucleic acids, carbohydrates, small molecules, or any other compound or composition which modulates the activity of HEM either by directly interacting with HEM or by acting on components of the biological pathway in which HEM participates.

The term “antibody” refers to intact immunoglobulin molecules as well as to fragments thereof, such as Fab, F(ab′)₂, and Fv fragments, which are capable of binding an epitopic determinant. Antibodies that bind HEM polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “antisense” refers to any composition capable of base-pairing with the “sense” (coding) strand of a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic HEM, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide sequence” and a “composition comprising a given amino acid sequence” refer broadly to any composition containing the given polynucleotide or amino acid sequence. The composition may comprise a dry formulation or an aqueous solution. Compositions comprising polynucleotide sequences encoding HEM or fragments of HEM may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (GCG, Madison Wis.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| | |
|---|---|
| Original Residue | Conservative Substitution |

| | |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

A “fragment” is a unique portion of HEM or the polynucleotide encoding HEM which is identical in sequence to but shorter in length than the parent sequence. A fragment may comprise up to the entire length of the defined sequence, minus one nucleotide/amino acid residue. For example, a fragment may comprise from 5 to 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:27-52 comprises a region of unique polynucleotide sequence that specifically identifies SEQ ID NO:27-52, for example, as distinct from any other sequence in the genome from which the fragment was obtained. A fragment of SEQ ID NO:27-52 is useful, for example, in hybridization and amplification technologies and in analogous methods that distinguish SEQ ID NO:27-52 from related polynucleotide sequences. The precise length of a fragment of SEQ ID NO:27-52 and the region of SEQ ID NO:27-52 to which the fragment corresponds are routinely determinable by one of ordinary skill in the art based on the intended purpose for the fragment.

A fragment of SEQ ID NO:1-26 is encoded by a fragment of SEQ ID NO:27-52. A fragment of SEQ ID NO:1-26 comprises a region of unique amino acid sequence that specifically identifies SEQ ID NO:1-26. For example, a fragment of SEQ ID NO:1-26 is useful as an immunogenic peptide for the development of antibodies that specifically recognize SEQ ID NO:1-26. The precise length of a fragment of SEQ ID NO:1-26 and the region of SEQ ID NO:1-26 to which the fragment corresponds are routinely determinable by one of ordinary skill in the art based on the intended purpose for the fragment.

A “full length” polynucleotide sequence is one containing at least a translation initiation codon (e.g., methionine) followed by an open reading frame and a translation termination codon. A “full length” polynucleotide sequence encodes a “full length” polypeptide sequence.

“Homology” refers to sequence similarity or, interchangeably, sequence identity, between two or more polynucleotide sequences or two or more polypeptide sequences.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P.M. Sharp (1989) CABIOS 5:151-153 and in Higgins, D. G. et al. (1992) CABIOS 8:189-191. For pairwise alignments of polynucleotide sequences, the default parameters are set as follows: Ktuple=2, gap penalty=5, window=4, and “diagonals saved”=4. The “weighted” residue weight table is selected as the default. Percent identity is reported by CLUSTAL V as the “percent similarity” between aligned polynucleotide sequences.

Alternatively, a suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/bl2.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr. 21, 2000) set at default parameters. Such default parameters may be, for example:

Matrix: BLOSUM62

Reward for match: 1

Penalty for mismatch: −2

Open Gap: 5 and Extension Gap: 2 penalties

Gap x drop-off: 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.12 (April-21-2000) with blastp set at default parameters. Such default parameters may be, for example:

Matrix: BLOSUM62

Open Gap: 11 and Extension Gap: 1 penalties

Gap x drop-off: 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. et al. (1989) Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acid sequences by virtue of the formation of hydrogen bonds between complementary bases. A hybridization complex may be formed in solution (e.g., C₀t or R₀t analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

The words “insertion” and “addition” refer to changes in an amino acid or nucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of HEM which is capable of eliciting an immune response when introduced into a living organism, for example, a mammal. The term “immunogenic fragment” also includes any polypeptide or oligopeptide fragment of HEM which is useful in any of the antibody production methods disclosed herein or known in the art.

The term “microarray” refers to an arrangement of a plurality of polynucleotides, polypeptides, or other chemical compounds on a substrate.

The terms “element” and “array element” refer to a polynucleotide, polypeptide, or other chemical compound having a unique and defined position on a microarray.

The term “modulate” refers to a change in the activity of HEM. For example, modulation may cause an increase or a decrease in protein activity, binding characteristics, or any other biological, functional, or immunological properties of HEM.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an HEM may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of HEM.

“Probe” refers to nucleic acid sequences encoding HEM, their complements, or fragments thereof, which are used to detect identical, allelic or related nucleic acid sequences. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid sequence, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in the references, for example Sambrook, J. et al. (1989) Molecular Cloning: A Laboratory Manual, 2^{nd} ed., vol. 1-3, Cold Spring Harbor Press, Plainview N.Y.; Ausubel, F. M. et al. (1987) Current Protocols in Molecular Biology, Greene Publ. Assoc. & Wiley-Intersciences, New York N.Y.; Innis, M. et al. (1990) PCR Protocols, A Guide to Methods and Applications, Academic Press, San Diego Calif. PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose.

The term “sample” is used in its broadest sense. A sample suspected of containing HEM, nucleic acids encoding HEM, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least 60% free, preferably at least 75% free, and most preferably at least 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May-07-1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternative splicing of exons during mRNA processing. The corresponding polypeptide may possess additional functional domains or lack domains that are present in the reference molecule. Species variants are polynucleotide sequences that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May-07-1999) set at default parameters. Such a pair of polypeptides may show, for example, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides.

The Invention

The invention is based on the discovery of new human enzyme molecules (HEM), the polynucleotides encoding HEM, and the use of these compositions for the diagnosis, treatment, or prevention of autoimmune/inflammation disorders, genetic disorders, neurological disorders, and cell proliferative disorders including cancer.

Table 1 summarizes the nomenclature for the full length polynucleotide and polypeptide sequences of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown.

Table 2 shows sequences with homology to the polypeptides of the invention as identified by BLAST analysis against the GenBank protein (genpept) database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (Genbank ID NO:) of the nearest GenBank homolog. Column 4 shows the probability score for the match between each polypeptide and its GenBank homolog. Column 5 shows the annotation of the GenBank homolog along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTIFS program of the GCG sequence analysis software package (Genetics Computer Group, Madison Wis.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are human enzyme molecules. For example, SEQ ID NO:5 is 84% identical, from residue Ml to residue E384, to Rattus norvegicus beta-alanine synthase (GenBank ID g203106) as determined by the Basic Local Alignment Search Tool (BLAST). (See Table 2.) The BLAST probability score is 0.0, which indicates the probability of obtaining the observed polypeptide sequence alignment by chance. SEQ ID NO:5 also contains a carbon-nitrogen hydrolase domain as determined by searching for statistically significant matches in the hidden Markov model (HMM)-based PFAM database of conserved protein family domains. (See Table 3.) Data from BLIMPS and additional BLAST analyses provide further corroborative evidence that SEQ ID NO:5 is a hydrolase. SEQ ID NO:1, SEQ ID NO:2, SEQ ID NO:3, SEQ ID NO:4, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, and SEQ ID NO:26 were analyzed and annotated in a similar manner. The algorithms and parameters for the analysis of SEQ ID NO:1-26 are described in Table 7.

As shown in Table 4, the full length polynucleotide sequences of the present invention were assembled using cDNA sequences or coding (exon) sequences derived from genomic DNA, or any combination of these two types of sequences. Columns 1 and 2 list the polynucleotide sequence identification number (Polynucleotide SEQ ID NO:) and the corresponding Incyte polynucleotide consensus sequence number (Incyte Polynucleotide ID) for each polynucleotide of the invention. Column 3 shows the length of each polynucleotide sequence in basepairs. Column 4 lists fragments of the polynucleotide sequences which are useful, for example, in hybridization or amplification technologies that identify SEQ ID NO:27-52 or that distinguish between SEQ ID NO:27-52 and related polynucleotide sequences. Column 5 shows identification numbers corresponding to cDNA sequences, coding sequences (exons) predicted from genomic DNA, and/or sequence assemblages comprised of both cDNA and genomic DNA. These sequences were used to assemble the full length polynucleotide sequences of the invention. Columns 6 and 7 of Table 4 show the nucleotide start (5′) and stop (3′) positions of the cDNA sequences in column 5 relative to their respective full length sequences.

The identification numbers in Column 5 of Table 4 may refer specifically, for example, to Incyte cDNAs along with their corresponding cDNA libraries. For example, 3540084H1 is the identification number of an Incyte cDNA sequence, and SEMVNOTO4 is the cDNA library from which it is derived. Incyte cDNAs for which cDNA libraries are not indicated were derived from pooled cDNA libraries (e.g., SXAD90083V1). Alternatively, the identification numbers in column 5 may refer to GenBank cDNAs or ESTs (e.g., g2795888) which contributed to the assembly of the full length polynucleotide sequences. Alternatively, the identification numbers in column 5 may refer to coding regions predicted by Genscan analysis of genomic DNA. The Genscan-predicted coding sequences may have been edited prior to assembly. (See Example IV.) Alternatively, the identification numbers in column 5 may refer to assemblages of both cDNA and Genscan-predicted exons brought together by an “exon stitching” algorithm. (See Example V.) Alternatively, the identification numbers in column 5 may refer to assemblages of both cDNA and Genscan-predicted exons brought together by an “exon-stretching” algorithm. (See Example V.) In some cases, Incyte cDNA coverage redundant with the sequence coverage shown in column 5 was obtained to confirm the final consensus polynucleotide sequence, but the relevant Incyte cDNA identification numbers are not shown.

Table 5 shows the representative cDNA libraries for those full length polynucleotide sequences which were assembled using Incyte cDNA sequences. The representative cDNA library is the Incyte cDNA library which is most frequently represented by the Incyte cDNA sequences which were used to assemble and confirm the above polynucleotide sequences. The tissues and vectors which were used to construct the cDNA libraries shown in Table 5 are described in Table 6.

The invention also encompasses HEM variants. A preferred HEM variant is one which has at least about 80%, or alternatively at least about 90%, or even at least about 95% amino acid sequence identity to the HEM amino acid sequence, and which contains at least one functional or structural characteristic of HEM.

The invention also encompasses polynucleotides which encode HEM. In a particular embodiment, the invention encompasses a polynucleotide sequence comprising a sequence selected from the group consisting of SEQ ID NO:27-52, which encodes HEM. The polynucleotide sequences of SEQ ID NO:27-52, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses a variant of a polynucleotide sequence encoding HEM. In particular, such a variant polynucleotide sequence will have at least about 70%, or alternatively at least about 85%, or even at least about 95% polynucleotide sequence identity to the polynucleotide sequence encoding HEM. A particular aspect of the invention encompasses a variant of a polynucleotide sequence comprising a sequence selected from the group consisting of SEQ ID NO:27-52 which has at least about 70%, or alternatively at least about 85%, or even at least about 95% polynucleotide sequence identity to a nucleic acid sequence selected from the group consisting of SEQ ID NO:27-52. Any one of the polynucleotide variants described above can encode an amino acid sequence which contains at least one functional or structural characteristic of HEM.

It will be appreciated by those skilled in the art that as a result of the degeneracy of the genetic code, a multitude of polynucleotide sequences encoding HEM, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring HEM, and all such variations are to be considered as being specifically disclosed.

Although nucleotide sequences which encode HEM and its variants are generally capable of hybridizing to the nucleotide sequence of the naturally occurring HEM under appropriately selected conditions of stringency, it may be advantageous to produce nucleotide sequences encoding HEM or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding HEM and its derivatives without altering the encoded amino acid sequences include the production of RNA transcripts having more desirable properties, such as a greater half-life, than transcripts produced from the naturally occurring sequence.

The invention also encompasses production of DNA sequences which encode HEM and HEM derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding HEM or any fragment thereof.

Also encompassed by the invention are polynucleotide sequences that are capable of hybridizing to the claimed polynucleotide sequences, and, in particular, to those shown in SEQ ID NO:27-52 and fragments thereof under various conditions of stringency. (See, e.g., Wahl, G. M. and S. L. Berger (1987) Methods Enzymol. 152:399-407; Kimmel, A. R. (1987) Methods Enzymol. 152:507-511.) Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Pharmacia Biotech, Piscataway N. J.), or combinations of polymerases and proofreading exonucleases such as those found in the ELONGASE amplification system (Life Technologies, Gaithersburg Md.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Molecular Dynamics, Sunnyvale Calif.), or other systems known in the art. The resulting sequences are analyzed using a variety of algorithms which are well known in the art. (See, e.g., Ausubel, F. M. (1997) Short Protocols in Molecular Biology, John Wiley & Sons, New York N.Y., unit 7.7; Meyers, R. A. (1995) Molecular Biology and Biotechnology, Wiley VCH, New York N.Y., pp. 856-853.)

The nucleic acid sequences encoding HEM may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector. (See, e.g., Sarkar, G. (1993) PCR Methods Applic. 2:318-322.) Another method, inverse PCR, uses primers that extend in divergent directions to amplify unknown sequence from a circularized template. The template is derived from restriction fragments comprising a known genomic locus and surrounding sequences. (See, e.g., Triglia, T. et al. (1988) Nucleic Acids Res. 16:8186.) A third method, capture PCR, involves PCR amplification of DNA fragments adjacent to known sequences in human and yeast artificial chromosome DNA. (See, e.g., Lagerstrom, M. et al. (1991) PCR Methods Applic. 1:111-119.) In this method, multiple restriction enzyme digestions and ligations may be used to insert an engineered double-stranded sequence into a region of unknown sequence before performing PCR. Other methods which may be used to retrieve unknown sequences are known in the art. (See, e.g., Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotide sequences or fragments thereof which encode HEM may be cloned in recombinant DNA molecules that direct expression of HEM, or fragments or functional equivalents thereof, in appropriate host cells. Due to the inherent degeneracy of the genetic code, other DNA sequences which encode substantially the same or a functionally equivalent amino acid sequence may be produced and used to express HEM.

The nucleotide sequences of the present invention can be engineered using methods generally known in the art in order to alter HEM-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of HEM, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, sequences encoding HEM may be synthesized, in whole or in part, using chemical methods well known in the art. (See, e.g., Caruthers, M. H. et al. (1980) Nucleic Acids Symp. Ser. 7:215-223; and Horn, T. et al. (1980) Nucleic Acids Symp. Ser. 7:225-232.) Alternatively, HEM itself or a fragment thereof may be synthesized using chemical methods. For example, peptide synthesis can be performed using various solution-phase or solid-phase techniques. (See, e.g., Creighton, T. (1984) Proteins, Structures and Molecular Properties, W H Freeman, New York N.Y., pp. 55-60; and Roberge, J. Y. et al. (1995) Science 269:202-204.) Automated synthesis may be achieved using the ABI 431A peptide synthesizer (Applied Biosystems). Additionally, the amino acid sequence of HEM, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography. (See, e.g., Chiez, R. M. and F. Z. Regnier (1990) Methods Enzymol. 182:392-421.) The composition of the synthetic peptides may be confirmed by amino acid analysis or by sequencing. (See, e.g., Creighton, supra, pp. 28-53.)

In order to express a biologically active HEM, the nucleotide sequences encoding HEM or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotide sequences encoding HEM. Such elements may vary in their strength and specificity. Specific initiation signals may also be used to achieve more efficient translation of sequences encoding HEM. Such signals include the ATG initiation codon and adjacent sequences, e.g. the Kozak sequence. In cases where sequences encoding HEM and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used. (See, e.g., Scharf, D. et al. (1994) Results Probl. Cell Differ. 20:125-162.) Methods which are well known to those skilled in the art may be used to construct expression vectors containing sequences encoding HEM and appropriate transcriptional and translational control elements. These methods include in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview N.Y., ch. 4, 8, and 16-17; Ausubel, F. M. et al. (1995) Current Protocols in Molecular Biology, John Wiley & Sons, New York N.Y., ch. 9, 13, and 16.)

A variety of expression vector/host systems may be utilized to contain and express sequences encoding HEM. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems. (See, e.g., Sambrook, supra; Ausubel, supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; and Harrington, J. J. et al. (1997) Nat. Genet. 15:345-355.) Expression vectors derived from retroviruses, adenoviruses, or herpes or vaccinia viruses, or from various bacterial plasmids, may be used for delivery of nucleotide sequences to the targeted organ, tissue, or cell population. (See, e.g., Di Nicola, M. et al. (1998) Cancer Gen. Ther. 5(6):350-356; Yu, M. et al. (1993) Proc. Natl. Acad. Sci. USA 90(13):6340-6344; Buller, R. M. et al. (1985) Nature 317(6040):813-815; McGregor, D. P. et al. (1994) Mol. Immunol. 31(3):219-226; and Verma, I. M. and N. Somia (1997) Nature 389:239-242.) The invention is not limited by the host cell employed.

In bacterial systems, a number of cloning and expression vectors may be selected depending upon the use intended for polynucleotide sequences encoding HEM. For example, routine cloning, subcloning, and propagation of polynucleotide sequences encoding HEM can be achieved using a multifunctional E. coli vector such as PBLUESCRIPT (Stratagene, La Jolla Calif.) or PSPORT1 plasmid (Life Technologies). Ligation of sequences encoding HEM into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence. (See, e.g., Van Heeke, G. and S. M. Schuster (1989) J. Biol. Chem. 264:5503-5509.) When large quantities of HEM are needed, e.g. for the production of antibodies, vectors which direct high level expression of HEM may be used. For example, vectors containing the strong, inducible SP6 or T7 bacteriophage promoter may be used.

Yeast expression systems may be used for production of HEM. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign sequences into the host genome for stable propagation. (See, e.g., Ausubel, 1995, supra; Bitter, G. A. et al. (1987) Methods Enzymol. 153:516-544; and Scorer, C. A. et al. (1994) Bio/Technology 12:181-184.)

Plant systems may also be used for expression of HEM. Transcription of sequences encoding HEM may be driven by viral promoters, e.g., the ³⁵S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used. (See, e.g., Coruzzi, G. et al. (1984) EMBO J. 3:1671-1680; Broglie, R. et al. (1984) Science 224:838-843; and Winter, J. et al. (1991) Results Probl. Cell Differ. 17:85-105.) These constructs can be introduced into plant cells by direct DNA transformation or pathogen-mediated transfection. (See, e.g., The McGraw Hill Yearbook of Science and Technology (1992) McGraw Hill, New York N.Y., pp. 191-196.)

In mammalian cells, a number of viral-based expression systems may be utilized. In cases where an adenovirus is used as an expression vector, sequences encoding HEM may be ligated into an adenovirus transcription/translation complex consisting of the late promoter and tripartite leader sequence. Insertion in a non-essential E1 or E3 region of the viral genome may be used to obtain infective virus which expresses HEM in host cells. (See, e.g., Logan, J. and T. Shenk (1984) Proc. Natl. Acad. Sci. USA 81:3655-3659.) In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes. (See, e.g., Harrington, J. J. et al. (1997) Nat. Genet. 15:345-355.)

For long term production of recombinant proteins in mammalian systems, stable expression of HEM in cell lines is preferred. For example, sequences encoding HEM can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr⁻ cells, respectively. (See, e.g., Wigler, M. et al. (1977) Cell 11:223-232; Lowy, I. et al. (1980) Cell 22:817-823.) Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively. (See, e.g., Wigler, M. et al. (1980) Proc. Natl. Acad. Sci. USA 77:3567-3570; Colbere-Garapin, F. et al. (1981) J. Mol. Biol. 150:1-14.) Additional selectable genes have been described, e.g., trpB and hisD, which alter cellular requirements for metabolites. (See, e.g., Hartman, S. C. and R. C. Mulligan (1988) Proc. Natl. Acad. Sci. USA 85:8047-8051.) Visible markers, e.g., anthocyanins, green fluorescent proteins (GFP; Clontech), β glucuronidase and its substrate β-glucuronide, or luciferase and its substrate luciferin may be used. These markers can be used not only to identify transformants, but also to quantify the amount of transient or stable protein expression attributable to a specific vector system. (See, e.g., Rhodes, C. A. (1995) Methods Mol. Biol. 55:121-131.)

Although the presence/absence of marker gene expression suggests that the gene of interest is also present, the presence and expression of the gene may need to be confirmed. For example, if the sequence encoding HEM is inserted within a marker gene sequence, transformed cells containing sequences encoding HEM can be identified by the absence of marker gene function. Alternatively, a marker gene can be placed in tandem with a sequence encoding HEM under the control of a single promoter. Expression of the marker gene in response to induction or selection usually indicates expression of the tandem gene as well.

In general, host cells that contain the nucleic acid sequence encoding HEM and that express HEM may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of HEM using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on HEM is preferred, but a competitive binding assay may be employed. These and other assays are well known in the art. (See, e.g., Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; and Pound, J. D. (1998) Immunochemical Protocols, Humana Press, Totowa N.J.)

A wide variety of labels and conjugation techniques are known by those skilled in the art and may be used in various nucleic acid and amino acid assays. Means for producing labeled hybridization or PCR probes for detecting sequences related to polynucleotides encoding HEM include oligolabeling, nick translation, end-labeling, or PCR amplification using a labeled nucleotide. Alternatively, the sequences encoding HEM, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Pharmacia Biotech, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding HEM may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode HEM may be designed to contain signal sequences which direct secretion of HEM through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant nucleic acid sequences encoding HEM may be ligated to a heterologous sequence resulting in translation of a fusion protein in any of the aforementioned host systems. For example, a chimeric HEM protein containing a heterologous moiety that can be recognized by a commercially available antibody may facilitate the screening of peptide libraries for inhibitors of HEM activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the HEM encoding sequence and the heterologous protein sequence, so that HEM may be cleaved away from the heterologous moiety following purification. Methods for fusion protein expression and purification are discussed in Ausubel (1995, supra, ch. 10). A variety of commercially available kits may also be used to facilitate expression and purification of fusion proteins.

In a further embodiment of the invention, synthesis of radiolabeled HEM may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

HEM of the present invention or fragments thereof may be used to screen for compounds that specifically bind to HEM. At least one and up to a plurality of test compounds may be screened for specific binding to HEM. Examples of test compounds include antibodies, oligonucleotides, proteins (e.g., receptors), or small molecules.

In one embodiment, the compound thus identified is closely related to the natural ligand of HEM, e.g., a ligand or fragment thereof, a natural substrate, a structural or functional mimetic, or a natural binding partner. (See, e.g., Coligan, J. E. et al. (1991) Current Protocols in Immunology 1(2): Chapter 5.) Similarly, the compound can be closely related to the natural receptor to which HEM binds, or to at least a fragment of the receptor, e.g., the ligand binding site. In either case, the compound can be rationally designed using known techniques. In one embodiment, screening for these compounds involves producing appropriate cells which express HEM, either as a secreted protein or on the cell membrane. Preferred cells include cells from mammals, yeast, Drosophila, or E. coli. Cells expressing HEM or cell membrane fractions which contain HEM are then contacted with a test compound and binding, stimulation, or inhibition of activity of either HEM or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with HEM, either in solution or affixed to a solid support, and detecting the binding of HEM to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

HEM of the present invention or fragments thereof may be used to screen for compounds that modulate the activity of HEM. Such compounds may include agonists, antagonists, or partial or inverse agonists. In one embodiment, an assay is performed under conditions permissive for HEM activity, wherein HEM is combined with at least one test compound, and the activity of HEM in the presence of a test compound is compared with the activity of HEM in the absence of the test compound. A change in the activity of HEM in the presence of the test compound is indicative of a compound that modulates the activity of HEM. Alternatively, a test compound is combined with an in vitro or cell-free system comprising HEM under conditions suitable for HEM activity, and the assay is performed. In either of these assays, a test compound which modulates the activity of HEM may do so indirectly and need not come in direct contact with the test compound. At least one and up to a plurality of test compounds may be screened.

In another embodiment, polynucleotides encoding HEM or their mammalian homologs may be “knocked out” in an animal model system using homologous recombination in embryonic stem (ES) cells. Such techniques are well known in the art and are useful for the generation of animal models of human disease. (See, e.g., U.S. Pat. No. 5,175,383 and U.S. Pat. No. 5,767,337.) For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding HEM may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding HEM can also be used to create “knockin” humanized animals (pigs) or transgenic animals (mice or rats) to model human disease. With knockin technology, a region of a polynucleotide encoding HEM is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress HEM, e.g., by secreting HEM in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of HEM and human enzyme molecules. In addition, the expression of HEM is closely associated with pancreatic, urogenital and cancerous tissues. Therefore, HEM appears to play a role in autoimmune/inflammation disorders, genetic disorders, neurological disorders, and cell proliferative disorders including cancer. In the treatment of disorders associated with increased HEM expression or activity, it is desirable to decrease the expression or activity of HEM. In the treatment of disorders associated with decreased HEM expression or activity, it is desirable to increase the expression or activity of HEM.

Therefore, in one embodiment, HEM or a fragment or derivative thereof may be administered to a subject to treat or prevent a disorder associated with decreased expression or activity of HEM. Examples of such disorders include, but are not limited to, an autoimmune/inflammation disorder such as acquired immunodeficiency syndrome (AIDS), actinic keratosis, Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, bursitis, cholecystitis, cirrhosis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, paroxysmal nocturnal hemoglobinuria, hepatitis, hypereosinophilia, irritable bowel syndrome, episodic lymphopenia with lymphocytotoxins, mixed connective tissue disease (MCTD), multiple sclerosis, myasthenia gravis, myocardial or pericardial inflammation, myelofibrosis, osteoarthritis, osteoporosis, pancreatitis, polycythemia vera, polymyositis, psoriasis, Reiter's syndrome, rheumatoid arthritis, scleroderma, Sjogren's syndrome, systemic anaphylaxis, systemic lupus erythematosus, systemic sclerosis, primary thrombocythemia, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, trauma and hematopoietic cancer including lymphoma, leukemia, and myeloma; a genetic disorder such as adrenoleukodystrophy, Alport's syndrome, choroideremia, Duchenne and Becker muscular dystrophy, Down's syndrome, cystic fibrosis, chronic granulomatous disease, Gaucher's disease, Huntington's chorea, Marfan's syndrome, muscular dystrophy, myotonic dystrophy, pycnodysostosis, Refsum's syndrome, retinoblastoma, sickle cell anemia, thalassemia, Werner syndrome, von Willebrand's disease, Wilms' tumor, Zellweger syndrome, peroxisomal acyl-CoA oxidase deficiency, peroxisomal thiolase deficiency, peroxisomal bifunctional protein deficiency, mitochondrial carnitine palmitoyl transferase and carnitine deficiency, mitochondrial very-long-chain acyl-CoA dehydrogenase deficiency, mitochondrial medium-chain acyl-CoA dehydrogenase deficiency, mitochondrial short-chain acyl-CoA dehydrogenase deficiency, mitochondrial electron transport flavoprotein and electron transport flavoprotein:ubiquinone oxidoreductase deficiency, mitochondrial trifunctional protein deficiency, and mitochondrial short-chain 3-hydroxyacyl-CoA dehydrogenase deficiency; a neurological disorder such as neonatal seizures, epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia; and cancers including adenocarcinoma, leukemia, lymphoma, melanoma, myeloma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus.

In another embodiment, a vector capable of expressing HEM or a fragment or derivative thereof may be administered to a subject to treat or prevent a disorder associated with decreased expression or activity of HEM including, but not limited to, those described above.

In a further embodiment, a composition comprising a substantially purified HEM in conjunction with a suitable pharmaceutical carrier may be administered to a subject to treat or prevent a disorder associated with decreased expression or activity of HEM including, but not limited to, those provided above.

In still another embodiment, an agonist which modulates the activity of HEM may be administered to a subject to treat or prevent a disorder associated with decreased expression or activity of HEM including, but not limited to, those listed above.

In a further embodiment, an antagonist of HEM may be administered to a subject to treat or prevent a disorder associated with increased expression or activity of HEM. Examples of such disorders include, but are not limited to, those autoimmune/inflammation disorders, genetic disorders, neurological disorders, and cell proliferative disorders including cancer described above. In one aspect, an antibody which specifically binds HEM may be used directly as an antagonist or indirectly as a targeting or delivery mechanism for bringing a pharmaceutical agent to cells or tissues which express HEM.

In an additional embodiment, a vector expressing the complement of the polynucleotide encoding HEM may be administered to a subject to treat or prevent a disorder associated with increased expression or activity of HEM including, but not limited to, those described above.

In other embodiments, any of the proteins, antagonists, antibodies, agonists, complementary sequences, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of HEM may be produced using methods which are generally known in the art. In particular, purified HEM may be used to produce antibodies or to screen libraries of pharmaceutical agents to identify those which specifically bind HEM. Antibodies to HEM may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are generally preferred for therapeutic use.

For the production of antibodies, various hosts including goats, rabbits, rats, mice, humans, and others may be immunized by injection with HEM or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to HEM have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of HEM amino acids may be fused with those of another protein, such as KLH, and antibodies to the chimeric molecule may be produced.

Monoclonal antibodies to HEM may be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:3142; Cote, R. J. et al. (1983) Proc. Natl. Acad. Sci. USA 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452-454.) Alternatively, techniques described for the production of single chain antibodies may be adapted, using methods known in the art, to produce HEM-specific single chain antibodies. Antibodies with related specificity, but of distinct idiotypic composition, may be generated by chain shuffling from random combinatorial immunoglobulin libraries. (See, e.g., Burton, D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments which contain specific binding sites for HEM may also be generated. For example, such fragments include, but are not limited to, F(ab′)₂ fragments produced by pepsin digestion of the antibody molecule and Fab fragments generated by reducing the disulfide bridges of the F(ab′)₂ fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays may be used for screening to identify antibodies having the desired specificity. Numerous protocols for competitive binding or imrnunoradiometric assays using either polyclonal or monoclonal antibodies with established specificities are well known in the art. Such immunoassays typically involve the measurement of complex formation between HEM and its specific antibody. A two-site, monoclonal-based immunoassay utilizing monoclonal antibodies reactive to two non-interfering HEM epitopes is generally used, but a competitive binding assay may also be employed (Pound, supra).

Various methods such as Scatchard analysis in conjunction with radioimmunoassay techniques may be used to assess the affinity of antibodies for HEM. Affinity is expressed as an association constant, Kₐ, which is defined as the molar concentration of HEM-antibody complex divided by the molar concentrations of free antigen and free antibody under equilibrium conditions. The Kₐ determined for a preparation of polyclonal antibodies, which are heterogeneous in their affinities for multiple HEM epitopes, represents the average affinity, or avidity, of the antibodies for HEM. The Kₐ determined for a preparation of monoclonal antibodies, which are monospecific for a particular HEM epitope, represents a true measure of affinity. High-affinity antibody preparations with Kₐ ranging from about 10⁹ to 10¹² L/mole are preferred for use in immunoassays in which the HEM-antibody complex must withstand rigorous manipulations. Low-affinity antibody preparations with Kₐ ranging from about 10⁶ to 10⁷ L/mole are preferred for use in immunopurification and similar procedures which ultimately require dissociation of HEM, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of HEM-antibody complexes. Procedures for evaluating antibody specificity, titer, and avidity, and guidelines for antibody quality and usage in various applications, are generally available. (See, e.g., Catty, supra, and Coligan et al. supra.)

In another embodiment of the invention, the polynucleotides encoding HEM, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding HEM. Such technology is well known in the art, and antisense oligonucleotides or larger fragments can be designed from various locations along the coding or control regions of sequences encoding HEM. (See, e.g., Agrawal, S., ed. (1996) Antisense Therapeutics, Humana Press Inc., Totawa N.J.)

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein. (See, e.g., Slater, J. E. et al. (1998) J. Allergy Cli. Immunol. 102(3):469-475; and Scanlon, K. J. et al. (1995) 9(13): 1288-1296.) Antisense sequences can also be introduced intracellularly through the use of viral vectors, such as retrovirus and adeno-associated virus vectors. (See, e.g., Miller, A. D. (1990) Blood 76:271; Ausubel, supra; Uckert, W. and W. Walther (1994) Pharmacol. Ther. 63(3):323-347.) Other gene delivery mechanisms include liposome-derived systems, artificial viral envelopes, and other systems known in the art. (See, e.g., Rossi, J. J. (1995) Br. Med. Bull. 51(1):217-225; Boado, R. J. et al. (1998) J. Pharm. Sci. 87(11):1308-1315; and Morris, M. C. et al. (1997) Nucleic Acids Res. 25(14):2730-2736.)

In another embodiment of the invention, polynucleotides encoding HEM may be used for somatic or germline gene therapy. Gene therapy may be performed to (i) correct a genetic deficiency (e.g., in the cases of severe combined immunodeficiency (SCID)—X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270;475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6;643-666; Crystal, R. G. et al. (1995) Hum. Gene Therapy 6:667-703), thalassamias, familial hypercholesterolemia, and hemophilia resulting from Factor VIII or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA. 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in HEM expression or regulation causes disease, the expression of HEM from an appropriate population of transduced cells may alleviate the clinical manifestations caused by the genetic deficiency.

In a further embodiment of the invention, diseases or disorders caused by deficiencies in HEM are treated by constructing mammalian expression vectors encoding HEM and introducing these vectors by mechanical means into HEM-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J-L. and H. Recipon (1998) Curr. Opin. Biotechnol. 9:445-450).

Expression vectors that may be effective for the expression of HEM include, but are not limited to, the PcDNA 3.1, EPITAG, PRCCMV2, PREP, PVAX vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). HEM may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and Blau, H. M. supra)), or (iii) a tissue-specific promoter or the native promoter of the endogenous gene encoding HEM from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to HEM expression are treated by constructing a retrovirus vector consisting of (i) the polynucleotide encoding HEM under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In the alternative, an adenovirus-based gene therapy delivery system is used to deliver polynucleotides encoding HEM to cells which have one or more genetic abnormalities with respect to the expression of HEM. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999) Annu. Rev. Nutr. 19:511-544 and Verma, I. M. and N. Somia (1997) Nature 18:389:239-242, both incorporated by reference herein.

In another alternative, a herpes-based, gene therapy delivery system is used to deliver polynucleotides encoding HEM to target cells which have one or more genetic abnormalities with respect to the expression of HEM. The use of herpes simplex virus (HSV)-based vectors may be especially valuable for introducing HEM to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999) J. Virol. 73:519-532 and Xu, H. et al. (1994) Dev. Biol. 163:152-161, hereby incorporated by reference. The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another alternative, an alphavirus (positive, single-stranded RNA virus) vector is used to deliver polynucleotides encoding HEM to target cells. The biology of the prototypic alphavirus, Semliki Forest Virus (SFV), has been studied extensively and gene transfer vectors have been based on the SFV genome (Garoff, H. and K.-J. Li (1998) Curr. Opin. Biotechnol. 9:464-469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for HEM into the alphavirus genome in place of the capsid-coding region results in the production of a large number of HEM-coding RNAs and the synthesis of high levels of HEM in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of HEM into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee, J. E. et al. (1994) in Huber, B. E. and B. I. Carr, Molecular and Immunologic Approaches, Futura Publishing, Mt. Kisco N.Y., pp. 163-177.) A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of sequences encoding HEM.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding HEM. Such DNA sequences may be incorporated into a wide variety of vectors with suitable RNA polymerase promoters such as 17 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

An additional embodiment of the invention encompasses a method for screening for a compound which is effective in altering expression of a polynucleotide encoding HEM. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences Effective compounds may alter polynucleotide expression by acting as either inhibitors or promoters of polynucleotide expression. Thus, in the treatment of disorders associated with increased HEM expression or activity, a compound which specifically inhibits expression of the polynucleotide encoding HEM may be therapeutically useful, and in the treament of disorders associated with decreased HEM expression or activity, a compound which specifically promotes expression of the polynucleotide encoding HEM may be therapeutically useful.

At least one, and up to a plurality, of test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding HEM is exposed to at least one test compound thus obtained. The sample may comprise, for example, an intact or permeabilized cell, or an in vitro cell-free or reconstituted biochemical system. Alterations in the expression of a polynucleotide encoding HEM are assayed by any method commonly known in the art. Typically, the expression of a specific nucleotide is detected by hybridization with a probe having a nucleotide sequence complementary to the sequence of the polynucleotide encoding HEM. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (See, e.g., Goldman, C. K. et al. (1997) Nat. Biotechnol. 15:462-466.)

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of HEM, antibodies to HEM, and mimetics, agonists, antagonists, or inhibitors of HEM.

The compositions utilized in this invention may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery has the advantage of administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising HEM or fragments thereof. For example, liposome preparations containing a cell-impermeable macromolecule may promote cell fusion and intracellular delivery of the macromolecule. Alternatively, HEM or a fragment thereof may be joined to a short cationic N-terminal portion from the HIV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example HEM or fragments thereof, antibodies of HEM, and agonists, antagonists or inhibitors of HEM, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅O/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind HEM may be used for the diagnosis of disorders characterized by expression of HEM, or in assays to monitor patients being treated with HEM or agonists, antagonists, or inhibitors of HEM. Antibodies useful for diagnostic purposes may be prepared in the same manner as described above for therapeutics. Diagnostic assays for HEM include methods which utilize the antibody and a label to detect HEM in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring HEM, including ELISAs, RIAs, and FACS, are known in the art and provide a basis for diagnosing altered or abnormal levels of HEM expression. Normal or standard values for HEM expression are established by combining body fluids or cell extracts taken from normal mammalian subjects, for example, human subjects, with antibodies to HEM under conditions suitable for complex formation. The amount of standard complex formation may be quantitated by various methods, such as photometric means. Quantities of HEM expressed in subject, control, and disease samples from biopsied tissues are compared with the standard values. Deviation between standard and subject values establishes the parameters for diagnosing disease.

In another embodiment of the invention, the polynucleotides encoding HEM may be used for diagnostic purposes. The polynucleotides which may be used include oligonucleotide sequences, complementary RNA and DNA molecules, and PNAs. The polynucleotides may be used to detect and quantify gene expression in biopsied tissues in which expression of HEM may be correlated with disease. The diagnostic assay may be used to determine absence, presence, and excess expression of HEM, and to monitor regulation of HEM levels during therapeutic intervention.

In one aspect, hybridization with PCR probes which are capable of detecting polynucleotide sequences, including genomic sequences, encoding HEM or closely related molecules may be used to identify nucleic acid sequences which encode HEM. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding HEM, allelic variants, or related sequences.

Probes may also be used for the detection of related sequences, and may have at least 50% sequence identity to any of the HEM encoding sequences. The hybridization probes of the subject invention may be DNA or RNA and may be derived from the sequence of SEQ ID NO:27-52 or from genomic sequences including promoters, enhancers, and introns of the HEM gene.

Means for producing specific hybridization probes for DNAs encoding HEM include the cloning of polynucleotide sequences encoding HEM or HEM derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotide sequences encoding HEM may be used for the diagnosis of disorders associated with expression of HEM. Examples of such disorders include, but are not limited to, an autoimmune/inflammation disorder such as acquired immunodeficiency syndrome (AIDS), actinic keratosis, Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, bursitis, cholecystitis, cirrhosis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, paroxysmal nocturnal hemoglobinuria, hepatitis, hypereosinophilia, irritable bowel syndrome, episodic lymphopenia with lymphocytotoxins, mixed connective tissue disease (MCTD), multiple sclerosis, myasthenia gravis, myocardial or pericardial inflammation, myelofibrosis, osteoarthritis, osteoporosis, pancreatitis, polycythemia vera, polymyositis, psoriasis, Reiter's syndrome, rheumatoid arthritis, scleroderma, Sjögren's syndrome, systemic anaphylaxis, systemic lupus erythematosus, systemic sclerosis, primary thrombocythemia, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, trauma and hematopoietic cancer including lymphoma, leukemia, and myeloma; a genetic disorder such as adrenoleukodystrophy, Alport's syndrome, choroideremia, Duchenne and Becker muscular dystrophy, Down's syndrome, cystic fibrosis, chronic granulomatous disease, Gaucher's disease, Huntington's chorea, Marfan's syndrome, muscular dystrophy, myotonic dystrophy, pycnodysostosis, Refsum's syndrome, retinoblastoma, sickle cell anemia, thalassemia, Werner syndrome, von Willebrand's disease, Wilms' tumor, Zellweger syndrome, peroxisomal acyl-CoA oxidase deficiency, peroxisomal thiolase deficiency, peroxisomal bifunctional protein deficiency, mitochondrial carnitine palmitoyl transferase and carnitine deficiency, mitochondrial very-long-chain acyl-CoA dehydrogenase deficiency, mitochondrial medium-chain acyl-CoA dehydrogenase deficiency, mitochondrial short-chain acyl-CoA dehydrogenase deficiency, mitochondrial electron transport flavoprotein and electron transport flavoprotein:ubiquinone oxidoreductase deficiency, mitochondrial trifunctional protein deficiency, and mitochondrial short-chain 3-hydroxyacyl-CoA dehydrogenase deficiency; a neurological disorder such as neonatal seizures, epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia; and cancers including adenocarcinoma, leukemia, lymphoma, melanoma, myeloma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. The polynucleotide sequences encoding HEM may be used in Southern or northern analysis, dot blot, or other membrane-based technologies; in PCR technologies; in dipstick, pin, and multiformat ELISA-like assays; and in microarrays utilizing fluids or tissues from patients to detect altered HEM expression. Such qualitative or quantitative methods are well known in the art.

In a particular aspect, the nucleotide sequences encoding HEM may be useful in assays that detect the presence of associated disorders, particularly those mentioned above. The nucleotide sequences encoding HEM may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of nucleotide sequences encoding HEM in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of HEM, a normal or standard profile for expression is established. This may be accomplished by combining body fluids or cell extracts taken from normal subjects, either animal or human, with a sequence, or a fragment thereof, encoding HEM, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Additional diagnostic uses for oligonucleotides designed from the sequences encoding HEM may involve the use of PCR. These oligomers may be chemically synthesized, generated enzymatically, or produced in vitro. Oligomers will preferably contain a fragment of a polynucleotide encoding HEM, or a fragment of a polynucleotide complementary to the polynucleotide encoding HEM, and will be employed under optimized conditions for identification of a specific gene or condition. Oligomers may also be employed under less stringent conditions for detection or quantification of closely related DNA or RNA sequences.

In a particular aspect, oligonucleotide primers derived from the polynucleotide sequences encoding HEM may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from the polynucleotide sequences encoding HEM are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (is SNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

Methods which may also be used to quantify the expression of HEM include radiolabeling or biotinylating nucleotides, coamplification of a control nucleic acid, and interpolating results from standard curves. (See, e.g., Melby, P. C. et al. (1993) J. Immunol. Methods 159:235-244; Duplaa, C. et al. (1993) Anal. Biochem. 212:229-236.) The speed of quantitation of multiple samples may be accelerated by running the assay in a high-throughput format where the oligomer or polynucleotide of interest is presented in various dilutions and a spectrophotometric or calorimetric response gives rapid quantitation.

In further embodiments, oligonucleotides or longer fragments derived from any of the polynucleotide sequences described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, HEM, fragments of HEM, or antibodies specific for HEM may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time. (See Seilhamer et al., “Comparative Gene Transcript Analysis,” U.S. Pat. No. 5,840,484, expressly incorporated by reference herein.) Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471, expressly incorporated by reference herein). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity. (See, for example, Press Release 00-02 from the National Institute of Environmental Health Sciences, released Feb. 29, 2000, available at http://www.niehs.nih.gov/oc/news/toxchip.htm.) Therefore, it is important and desirable in toxicological screening using toxicant signatures to include all expressed gene sequences.

In one embodiment, the toxicity of a test compound is assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another particular embodiment relates to the use of the polypeptide sequences of the present invention to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of the present invention. In some cases, further sequence data may be obtained for definitive protein identification.

A proteomic profile may also be generated using antibodies specific for HEM to quantify the levels of HEM expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art. (See, e.g., Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.) Various types of microarrays are well known and thoroughly described in DNA Microarrays: A Practical Approach, M. Schena, ed. (1999) Oxford University Press, London, hereby expressly incorporated by reference.

In another embodiment of the invention, nucleic acid sequences encoding HEM may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries. (See, e.g., Harrington, J. J. et al. (1997) Nat. Genet. 15:345-355; Price, C. M. (1993) Blood Rev. 7:127-134; and Trask, B. J. (1991) Trends Genet. 7:149-154.) Once mapped, the nucleic acid sequences of the invention may be used to develop genetic linkage maps, for example, which correlate the inheritance of a disease state with the inheritance of a particular chromosome region or restriction fragment length polymorphism (RFLP). (See, for example, Lander, E. S. and D. Botstein (1986) Proc. Natl. Acad. Sci. USA 83:7353-7357.)

Fluorescent in situ hybridization (FISH) may be correlated with other physical and genetic map data. (See, e.g., Heinz-Ulrich, et al. (1995) in Meyers, supra, pp. 965-968.) Examples of genetic map data can be found in various scientific journals or at the Online Mendelian Inheritance in Man (OMIM) World Wide Web site. Correlation between the location of the gene encoding HEM on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation. (See, e.g., Gatti, R. A. et al. (1988) Nature 336:577-580.) The nucleotide sequence of the instant invention may also be used to detect differences in the chromosomal location due to translocation, inversion, etc., among normal, carrier, or affected individuals.

In another embodiment of the invention, HEM, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between HEM and the agent being tested may be measured.

Another technique for drug screening provides for high throughput screening of compounds having suitable binding affinity to the protein of interest. (See, e.g., Geysen, et al. (1984) PCT application WO84/03564.) In this method, large numbers of different small test compounds are synthesized on a solid substrate. The test compounds are reacted with HEM, or fragments thereof, and washed. Bound HEM is then detected by methods well known in the art. Purified HEM can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding HEM specifically compete with a test compound for binding HEM. In this manner, antibodies can be used to detect the presence of any peptide which shares one or more antigenic determinants with HEM.

In additional embodiments, the nucleotide sequences which encode HEM may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following embodiments are, therefore, to be construed as merely illustrative, and not limitative of the remainder of the disclosure in any way whatsoever.

The disclosures of all patents, applications and publications, mentioned above and below, in particular U.S. Ser. No. 60/186,307, U.S. Ser. No. 60/193,578, and U.S. Ser. No. 60/192,532, are expressly incorporated by reference herein.

### EXAMPLES

I. Construction of cDNA Libraries

Incyte cDNAs were derived from cDNA libraries described in the LIFESEQ GOLD database (Incyte Genomics, Palo Alto Calif.) and shown in Table 4, column 5. Some tissues were homogenized and lysed in guanidinium isothiocyanate, while others were homogenized and lysed in phenol or in a suitable mixture of denaturants, such as TRIZOL (Life Technologies), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+ RNA was isolated using oligo d(T)-coupled paramagnetic particles (Promega), OLIGOTEX latex particles (QIAGEN, Chatsworth Calif.), or an OLIGOTEX rRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Life Technologies), using the recommended procedures or similar methods known in the art. (See, e.g., Ausubel, 1997, supra, units 5.1-6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Pharmacia Biotech) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Life Technologies), PcDNA2.1 plasmid (Invitrogen, Carlsbad Calif.), PBK-CMV plasmid (Stratagene), or pINCY (Incyte Genomics, Palo Alto Calif.), or derivatives thereof. Recombinant plasmids were transformed into competent E. coli cells including XL1-Blue, XLI-BlueMRF, or SOLR from Stratagene or DH5α, DH10B, or ElectroMAX DH10B from Life Technologies.

II. Isolation of cDNA Clones

Plasmids obtained as described in Example I were recovered from host cells by in vivo excision using the UNIZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Pharmacia Biotech or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Molecular Dynamics); the ABI PRISM 373 or 377 sequencing system (Applied Biosystems) in conjunction with standard ABI protocols and base calling software; or other sequence analysis systems known in the art. Reading frames within the cDNA sequences were identified using standard methods (reviewed in Ausubel, 1997, supra, unit 7.7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM, and hidden Markov model (HMM)-based protein family databases such as PFAM. (HMM is a probabilistic approach which analyzes consensus primary structures of gene families. See, for example, Eddy, S. R. (1996) Curr. Opin. Struct. Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide of the invention may begin at any of the methionine residues of the full length translated polypeptide. Full length polypeptide sequences were subsequently analyzed by querying against databases such as the GenBank protein databases (genpept), SwissProt, BLOCKS, PRINTS, DOMO, PRODOM, Prosite, and hidden Markov model (HMM)-based protein family databases such as PFAM. Full length polynucleotide sequences are also analyzed using MACDNASIS PRO software (Hitachi Software Engineering, South San Francisco Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:27-52. Fragments from about 20 to about 4000 nucleotides which are useful in hybridization and amplification technologies are described in Table 4, column 4.

IV. Identification and Editing of Coding Sequences from Genomic DNA

Putative human enzyme molecules were initially identified by running the Genscan gene identification program against public genomic sequence databases (e.g., gbpri and gbhtg). Genscan is a general-purpose gene identification program which analyzes genomic DNA sequences from a variety of organisms (See Burge, C. and S. Karlin (1997) J. Mol. Biol. 268:78-94, and Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once was set to 30 kb. To determine which of these Genscan predicted cDNA sequences encode human enzyme molecules, the encoded polypeptides were analyzed by querying against PFAM models for human enzyme molecules. Potential human enzyme molecules were also identified by homology to Incyte cDNA sequences that had been annotated as human enzyme molecules. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example m. Alternatively, full length polynucleotide sequences were derived entirely from edited or unedited Genscan-predicted coding sequences.

V. Assembly of Genomic Sequence Data With cDNA Sequence Data

“Stitched” Sequences

Partial cDNA sequences were extended with exons predicted by the Genscan gene identification program described in Example IV. Partial cDNAs assembled as described in Example III were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example III were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of HEM Encoding Polynucleotides

The sequences which were used to assemble SEQ ID NO:27-52 were compared with sequences from the Incyte LIFESEQ database and public domain databases using BLAST and other implementations of the Smith-Waterman algorithm. Sequences from these databases that matched SEQ ID NO:27-52 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Genethon were used to determine if any of the clustered sequences had been previously mapped. Inclusion of a mapped sequence in a cluster resulted in the assignment of all sequences of that cluster, including its particular SEQ ID NO: to that map location.

Map locations are represented by ranges, or intervals, or human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Genethon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

In this manner, SEQ ID NO:32 was mapped to chromosome 20 within the interval from 11.0 to 20.9 centimorgans. SEQ ID NO:35 was mapped to chromosome 2 within the interval from 175.0 to 180.6 centiMorgans and within the interval from 190.8 to 196.8 centiMorgans. SEQ ID NO:41 was mapped to chromosome 1 within the interval from 235.8 to 237.2 centimorgans and within the interval from 243.3 to 245.2 centiMorgans. SEQ ID NO:47 was mapped to chromosome 2 within the interval from 118.0 to 127.4 centiMorgans. More than one map location is reported for SEQ ID NO:35 and SEQ ID NO:41, indicating that sequences having different map locations were assembled into a single cluster. This situation occurs, for example, when sequences having strong similarity, but not complete identity, are assembled into a single cluster.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; Ausubel (1995) supra, ch. 4 and 16.)

Analogous computer techniques applying BLAST were used to search for identical or related molecules in cDNA databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotide sequences encoding HEM are analyzed with respect to the tissue sources from which they were derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding HEM. cDNA sequences and cDNA library/tissue information are found in the LIFESEQ GOLD database (Incyte Genomics, Palo Alto Calif.).

VIII. Extension of HEM Encoding Polynucleotides

Full length polynucleotide sequences were also produced by extension of an appropriate fragment of the full length molecule using oligonucleotide primers designed from this fragment. One primer. was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 mmol of each primer, reaction buffer containing Mg²⁺, (NH₄)₂SO₄, and 2-mercaptoethanol, Taq DNA polymerase (Amersham Pharmacia Biotech), ELONGASE enzyme (Life Technologies), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1×TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Pharmacia Biotech). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Pharmacia Biotech), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2×carb liquid media.

The cells were lysed, and DNA was amplified by PCR using Taq DNA polymerase (Amersham Pharmacia Biotech) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Pharmacia Biotech) or the ABI PRISM BIGDYE Terminator cycle sequencing ready reaction kit (Applied Biosystems).

In like manner, full length polynucleotide sequences are verified using the above procedure or are used to obtain 5′ regulatory sequences using the above procedure along with oligonucleotides designed for such extension, and an appropriate genomic library.

IX. Labeling and Use of Individual Hybridization Probes

Hybridization probes derived from SEQ ID NO:27-52 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Pharmacia Biotech), and T4 polynucleotide kinase (DuPont NEN, Boston Mass.). The labeled oligonucleotides are substantially purified using a SEPHADEX G-25 superfine size exclusion dextran bead column (Amersham Pharmacia Biotech). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1×saline sodium citrate and 0.5% sodium dodecyl sulfate. Hybridization patterns are visualized using autoradiography or an alternative imaging means and compared.

X. Microarrays

The linkage or synthesis of array elements upon a microarray can be achieved utilizing photolithography, piezoelectric printing (inkjet printing, See, e.g., Baldeschweiler, supra.), mechanical microspotting technologies, and derivatives thereof. The substrate in each of the aforementioned technologies should be uniform and solid with a non-porous surface (Schena (1999), supra). Suggested substrates include silicon, silica, glass slides, glass chips, and silicon wafers. Alternatively, a procedure analogous to a dot or slot blot may also be used to arrange and link elements to the surface of a substrate using thermal, V, chemical, or mechanical bonding procedures. A typical array may be produced using available methods and machines well known to those of ordinary skill in the art and may contain any appropriate number of elements. (See, e.g., Schena, M. et al. (1995) Science 270:467-470; Shalon, D. et al. (1996) Genome Res. 6:639-645; Marshall, A. and J. Hodgson (1998) Nat. Biotechnol. 16:27-31.)

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation Total RNA is isolated from tissue samples using the guanidinium thiocyanate method and poly(A)⁺ RNA is purified using the oligo-(dT) cellulose method. Each poly(A)+ RNA sample is reverse transcribed using MMLV reverse-transcriptase, 0.05 pg/μl oligo-(dT) primer (21mer), 1× first strand buffer, 0.03 units/μl RNase inhibitor, 500 μM dATP, 500 μM dGTP, 500 μM dTTP, 40 μM dCTP, 40 μM dCTP-Cy3 (BDS) or dCTP-Cy5 (Amersham Pharmacia Biotech). The reverse transcription reaction is performed in a 25 ml volume containing 200 ng poly(A)⁺ RNA with GEMBRIGHT kits (Incyte). Specific control poly(A)+ RNAs are synthesized by in vitro transcription from non-coding yeast genomic DNA. After incubation at 370 C for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (CLONTECH Laboratories, Inc. (CLONTECH), Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Pharmacia Biotech).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 10° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20×microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte).

XI. Complementary Polynucleotides

Sequences complementary to the HEM-encoding sequences, or any parts thereof, are used to detect, decrease, or inhibit expression of naturally occurring HEM. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of HEM. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the HEM-encoding transcript.

XII. Expression of HEM

Expression and purification of HEM is achieved using bacterial or virus-based expression systems. For expression of HEM in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express HEM upon induction with isopropyl beta-D-thiogalactopyranoside (IPTG). Expression of HEM in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding HEM by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus. (See Engelhard, E. K. et al. (1994) Proc. Natl. Acad. Sci. USA 91:3224-3227; Sandig, V. et al. (1996) Hum. Gene Ther. 7:1937-1945.)

In most expression systems, HEM is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Pharmacia Biotech). Following purification, the GST moiety can be proteolytically cleaved from HEM at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel (1995, supra, ch. 10 and 16). Purified HEM obtained by these methods can be used directly in the assays shown in Examples XVI and XVII, where applicable.

XIII. Functional Assays

HEM function is assessed by expressing the sequences encoding HEM at physiologically elevated levels in mammalian cell culture systems. cDNA is subcloned into a mammalian expression vector containing a strong promoter that drives high levels of cDNA expression. Vectors of choice include PCMV SPORT (Life Technologies) and PCR3.1 (Invitrogen, Carlsbad Calif.), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York N.Y.

The influence of HEM on gene expression can be assessed using highly purified populations of cells transfected with sequences encoding HEM and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding HEM and other genes of interest can be analyzed by northern analysis or microarray techniques.

XIV. Production of HEM Specific Antibodies

HEM substantially purified using polyacrylamide gel electrophoresis (PAGE; see, e.g., Harrington, M. G. (1990) Methods Enzymol. 182:488495), or other purification techniques, is used to immunize rabbits and to produce antibodies using standard protocols.

Alternatively, the HEM amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art. (See, e.g., Ausubel, 1995, supra, ch. 11.)

Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity. (See, e.g., Ausubel, 1995, supra.) Rabbits are immunized with the oligopeptide-KLH complex in complete Freund's adjuvant. Resulting antisera are tested for antipeptide and anti-HEM activity by, for example, binding the peptide or HEM to a substrate, blocking with 1% BSA, reacting with rabbit antisera, washing, and reacting with radio-iodinated goat anti-rabbit IgG.

XV. Purification of Naturally Occurring HEM Using Specific Antibodies

Naturally occurring or recombinant HEM is substantially purified by immunoaffinity chromatography using antibodies specific for HEM. An immunoaffinity column is constructed by covalently coupling anti-HEM antibody to an activated chromatographic resin, such as CNBr-activated SEPHAROSE (Amersham Pharmacia Biotech). After the coupling, the resin is blocked and washed according to the manufacturer's instructions.

Media containing HEM are passed over the immunoaffinity column, and the column is washed under conditions that allow the preferential absorbance of HEM (e.g., high ionic strength buffers in the presence of detergent). The column is eluted under conditions that disrupt antibody/HEM binding (e.g., a buffer of pH 2 to pH 3, or a high concentration of a chaotrope, such as urea or thiocyanate ion), and HEM is collected.

XVI. Identification of Molecules Which Interact With HEM

HEM, or biologically active fragments thereof, are labeled with ¹²⁵I Bolton-Hunter reagent. (See, e.g., Bolton A. E. and W. M. Hunter (1973) Biochem. J. 133:529-539.) Candidate molecules previously arrayed in the wells of a multi-well plate are incubated with the labeled HEM, washed, and any wells with labeled HEM complex are assayed. Data obtained using different concentrations of HEM are used to calculate values for the number, affinity, and association of HEM with the candidate molecules.

Alternatively, molecules interacting with HEM are analyzed using the yeast two-hybrid system as described in Fields, S. and O. Song (1989) Nature 340:245-246, or using commercially available kits based on the two-hybrid system, such as the MATCHMAKER system (Clontech).

HEM may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVII. Demonstration of HEM Activity

Cofactor Biosynthetic Enzyme Activity

The molybdopterin synthase sulfurylase activity of HEM is measured, for example, by transforming an Aspergillus nidulans cizxF mutant with a suitable expression vector containing the polynucleotide sequence of HEM and observing whether the A. nidulans cells are able to grow on nitrate or hypoxanthine-containing media (see Appleyard, M. et al., supra). Since molybdopterin synthase sulfurylase activity is absent in A. nidulans cnxF mutants, and this activity is necessary for utilization of nitrate or hypoxanthine, the rescue of these mutants with an expression vector containing the polynucleotide sequence of HEM indicates that HEM has molybdopterin synthase sulfurylase activity. Also, the levels of molybdopterin and intermediates in the biosynthetic pathway are measured using HPLC analysis (see Appleyard, M. et al, supra).

Isomerase Activity

Isomerase activity of HEM is demonstrated through a variety of specific enzyme assays, some of which are outlined below.

Peptidyl Prolyl cis-trans Isomerase Activity

HEM peptidyl prolyl cis-trans isomerase activity can be assayed as described (Rahfeld, J. U. et al. (1994) FEBS Lett. 352:180-184). The assay is performed at 10° C. in 35 mM HEPES buffer, pH 7.8, containing chymotrypsin (0.5 mg/ml) and HEM at a variety of concentrations. In this assay, the substrate is a peptide containing four hydrophobic residues. The peptide contains a succinate group at the N-terminus and a nitroanilide group at the C-terminus. The substrate is in equilibrium with respect to the prolyl bond, with 80-95% in trans and 5-20% in cis conformation. An aliquot (2 μl) of the substrate dissolved in dimethyl sulfoxide (10 mg/ml) is added to the reaction mixture described above. Only the cis isomer of the substrate is a substrate for cleavage by chymotrypsin. Thus, as the substrate is isomerized by HEM, the product is cleaved by chymotrypsin to produce 4-nitroanilide, which is detected by its absorbance at 390 nm. 4-Nitroanilide appears in a time-dependent and an HEM concentration-dependent manner.

Alternatively, peptidyl prolyl cis-trans isomerase activity of HEM can be assayed using a chromogenic peptide in a coupled assay with chymotrypsin (Fischer, G. et al. (1984) Biomed. Biochim. Acta 43:1101-1111).

Thioredoxin Activity

HEM thioredoxin activity is assayed as described (Luthman, M. (1982) Biochemistry 21:6628-6633). Thioredoxins catalyze the formation of disulfide bonds and regulate the redox environment in cells to enable the necessary thiol:disulfide exchanges. One way to measure the thiol:disulfide exchange is by measuring the reduction of insulin in a mixture containing 0.1M potassium phosphate, pH 7.0, 2 mM EDTA, 0.16 μM insulin, 0.33 mM DTT, and 0.48 mM NADPH. Different concentrations of HEM are added to the mixture, and the reaction rate is followed by monitoring the oxidation of NADPH at 340 nM.

Transferase Activity

HEM transferase activity is measured through a methyl transferase assay in which the transfer of radiolabeled methyl groups between a donor substrate and an acceptor substrate is measured (Bokar, J. A. et al. (1994) J. Biol. Chem. 269:17697-17704). Reaction mixtures (50 μl final volume) contain 15 mM HEPES, pH 7.9, 1.5 mM MgCl₂, 10 mM dithiothreitol, 3% polyvinylalcohol, donor substrate (1.5 μCi [methyl-³H]AdoMet (0.375 μM AdoMet) (DuPont-NEN)), 0.6 μg HEM, and acceptor substrate (0.4 μg [³⁵S]RNA or 6-mercaptopurine (6-MP) to 1 mM final concentration). Reaction mixtures are incubated at 30° C. for 30 minutes, then 65° C. for 5 minutes. The products are separated by chromatography or electrophoresis and the level of methyl transferase activity is determined by quantification of methyl-3H—RNA or methyl-3H-6-MP recovery.

Hydrolase Activity

For purposes of example, an assay measuring the β-glucosidase activity of an HEM molecule is described. Varying amounts of HEM are incubated with 1 mM 4-nitrophenyl β-D-glycopyranoside (a substrate) in 50 mM sodium acetate buffer, pH 5.0, for various times (typically 1-5 minutes) at 37° C. The reaction is terminated by heating to 100° C. for 2 minutes. The absorbance is measured spectrophotometrically at 410 nm, and the change in absorbance is proportional to the activity of HEM in the sample. (Hrmova, M. et al. (1998) J. Biol. Chem. 273:11134-11143.)

Various modifications and variations of the described methods and systems of the invention will be apparent to those skilled in the art without departing from the scope and spirit of the invention. Although the invention has been described in connection with certain embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

TABLE 1

| | | Incyte | | Incyte |
|---|---|---|---|---|
| Incyte | Polypeptide | Polypeptide | Polynucleotide | Polynucleotide |
| Project ID | SEQ ID NO: | ID | SEQ ID NO: | ID |
| 2783442 | 1 | 2783442CD1 | 27 | 2783442CB1 |
| 2116390 | 2 | 2116390CD1 | 28 | 2116390CB1 |
| 5785224 | 3 | 5785224CD1 | 29 | 5785224CB1 |
| 1870996 | 4 | 1870996CD1 | 30 | 1870996CB1 |
| 138841 | 5 | 138841CD1 | 31 | 138841CB1 |
| 1485405 | 6 | 1485405CD1 | 32 | 1485405CB1 |
| 2024617 | 7 | 2024617CD1 | 33 | 2024617CB1 |
| 4721827 | 8 | 4721827CD1 | 34 | 4721827CB1 |
| 5406614 | 9 | 5406614CD1 | 35 | 5406614CB1 |
| 1252792 | 10 | 1252792CD1 | 36 | 1252792CB1 |
| 1299305 | 11 | 1299305CD1 | 37 | 1299305CB1 |
| 1385190 | 12 | 1385190CD1 | 38 | 1385190CB1 |
| 2133162 | 13 | 2133162CD1 | 39 | 2133162CB1 |
| 254567 | 14 | 254567CD1 | 40 | 254567CB1 |
| 3903488 | 15 | 3903488CD1 | 41 | 3903488CB1 |
| 5391816 | 16 | 5391816CD1 | 42 | 5391816CB1 |
| 5886989 | 17 | 5886989CD1 | 43 | 5886989CB1 |
| 723432 | 18 | 723432CD1 | 44 | 723432CB1 |
| 1332963 | 19 | 1332963CD1 | 45 | 1332963CB1 |
| 1559410 | 20 | 1559410CD1 | 46 | 1559410CB1 |
| 1752587 | 21 | 1752587CD1 | 47 | 1752587CB1 |
| 1911509 | 22 | 1911509CD1 | 48 | 1911509CB1 |
| 2210170 | 23 | 2210170CD1 | 49 | 2210170CB1 |
| 234664 | 24 | 234664CD1 | 50 | 234664CB1 |
| 2884114 | 25 | 2884114CD1 | 51 | 2884114CB1 |
| 4103559 | 26 | 4103559CD1 | 52 | 4103559CB1 |

TABLE 2

| Polypeptide | Incyte | GenBank | Probability | GenBank |
|---|---|---|---|---|
| SEQ ID NO: | Polypeptide ID | ID NO: | score | Homolog |
| 1 | 2783442CD1 | g1825606 | 1.00E−130 | similar to molybdopterin |
| | | | | biosynthesis MOEB proteins |
| | | | | [Caenorhabditis elegans] |
| | | | | Wilson, R. et al. (1994) Nature |
| | | | | 368:32-38 |
| 2 | 2116390CD1 | g2984222 | 1.2e−12 | Fuculose-1-phosphate aldolase |
| | | | | [Aquifex aeolicus] |
| | | | | Deckert, G. et al. (1998) Nature |
| | | | | 392:353-358 |
| | | g10177247 | 1.00E−55 | contains similarity to enolase- |
| | | | | phosphatase˜gene_id:K19P17.1 |
| | | | | [Arabidopsis thaliana] |
| | | | | Nakamura, Y. et al. (1997) DNA Res. |
| | | | | 4:401-414 |
| 3 | 5785224CD1 | g1799782 | 2e−61 | Bile acid-inducible operon Protein |
| | | | | F [Escherichia coli] |
| | | | | Yamamoto, Y. et al. (1997) DNA Res. |
| | | | | 4:91-113 |
| 4 | 1870996CD1 | g1788642 | 1.00E−19 | putative sugar nucleotide epimerase |
| | | | | [Escherichia coli K12] |
| | | | | Blattner, F. R. et al. (1997) |
| | | | | Science 277:1453-1474 |
| 5 | 138841CD1 | g203106 | 0 | beta-alanine synthase [Rattus |
| | | | | norvegicus] |
| | | | | Kvalnes-Krick, K. L. et al. (1993) |
| | | | | J. Biol. Chem. 268:5686-5693 |
| 6 | 1485405CD1 | g19137 | 6.4e−33 | Asparaginase [Lupinus arboreus] |
| | | | | Lough, T. J. et al. (1992) Plant Mol |
| | | | | Biol 19:391-399 |
| 7 | 2024617CD1 | g1196814 | 2.70E−278 | L-glutamine amidohydrolase [Rattus |
| | | | | norvegicus] |
| | | | | Smith, E. M. et al. (1990) J. Biol. |
| | | | | Chem. 265:10631-10636 |
| 8 | 4721827CD1 | g11546051 | 1.00E−115 | dJ1049G16.1.1 (KIAA1247 (similar to |
| | | | | glucosainine-6-sulfatases and |
| | | | | KIAA1077)) [Homo sapiens] |
| 9 | 5406614CD1 | g5524927 | 1.30E−161 | deoxyribonuclease III (DNase III) |
| | | | | [Homo sapiens] |
| | | | | Hoss, M. et al. (1999) EMBO J. |
| | | | | 18:3868-3875 |
| 10 | 1252792CD1 | g7576450 | 0 | ALEX1 [Homo sapiens] |
| | | | | Kurochkin, I. V. et al. (2001) |
| | | | | Biochem. Biophys. Res. Commun. |
| | | | | 280:340-347 |
| 11 | 1299305CD1 | g11545670 | 0 | serine beta lactamase-like protein |
| | | | | LACT-1 [Mus musculus] |
| 15 | 3903488CD1 | g4103135 | 6.00E−66 | putative transcription factor Fet5 |
| | | | | [Schizosaccharomyces pombe] |
| | | | | Shpakovskii, G. V. et a1. (1997) |
| | | | | Bioorg. Khim. 23:234-237 |
| | | g2330815 | 1.7e−86 | Purine nucleotide binding protein |
| | | | | fet5 [Schizosacoharomyces pombe] |
| 16 | 5391816CD1 | g3822036 | 1.40E−15 | “endo-1,3-1,4-beta-D-glucanase [Zea |
| | | | | mays]” |
| | | | | Thomas, B. R. et al. (1998) Plant |
| | | | | Physiol. 117:1525 |
| 18 | 723432CD1 | g6459813 | 2.20E−61 | ArgE/DapE/Acy1 family aminoacylase |
| | | | | protein [Deinococcus radiodurans] |
| | | | | White, O. et al. (1999) Science |
| | | | | 286:1571-1577 |
| 19 | 1332963CD1 | g10173877 | 3.00E−82 | (5-methylaminomethyl-2- |
| | | | | thiouridylate)-methyl transferase |
| | | | | [Bacillus halodurans] |
| | | | | Takami, H. et al. (1999) |
| | | | | Extremophiles 3:21-28 |
| | | g3328701 | 2.70E−69 | PP-loop superfamily ATPase |
| | | | | [Chlamydia trachomatis] |
| | | | | Stephens, R. S. et al. (1998) |
| | | | | Science 282:754-759 |
| 20 | 15594100D1 | g1943807 | 8.00E−57 | weak similarity to ATPases |
| | | | | [Caenorhabditis elegans] |
| | | | | The C. elegans Genome Sequencing |
| | | | | Consortium (1996) Science 282:2012- |
| | | | | 2018 |
| 21 | 1752587CD1 | g2225906 | 2.20E−68 | PIG-L deacetylase [Rattus |
| | | | | norvegicus] |
| | | | | Nakamura, N. et al. (1997) J. Biol. |
| | | | | Chem. 272:15834-45840 |
| 22 | 1911509CD1 | g1825778 | 1.10E−57 | similar to acetyltransferases |
| | | | | [Caenorhabditis elegans] |
| | | | | Wilson, R. et al. (1994) Nature |
| | | | | 368:32-38 |
| 23 | 2210170CD1 | g2853081 | 5.00E−58 | ATP binding protein-like |
| | | | | [Arabidopsis thaliana] |
| 24 | 234664CD1 | g1101009 | 5.00E−26 | endothelin converting enzyme-2 “[Bos |
| | | | | taurus]” |
| | | | | Emoto, N. et al. (1995) J. Biol. |
| | | | | Chem. 270:15262-15268 |
| 25 | 2884114CD1 | g2318131 | 3.00E−90 | histone deacetylase [Arabidopsis |
| | | | | thaliana] |
| | | g8132878 | 0 | histone deacetylase 8; HDAC8 [Homo |
| | | | | sapiens] |
| 26 | 4103559CD1 | g3098603 | 1.30E−53 | katanin p60 subunit |
| | | | | [Strongylocentrotus purpuratus] |

TABLE 3

| SEQ | Incyte | Amino | Potential | Potential | | Analytical |
|---|---|---|---|---|---|---|
| ID | Polypeptide | Acid | Phosphorylation | Glycosylation | Signature Sequences, | Methods and |
| NO: | ID | Residues | Sites | Sites | Domains and Motifs | Databases |
| 1 | 2783442CD1 | 404 | S66 T152 S180 | N150 N356 | ThiF_family (repeated domain in | HMMER-PFAM |
| | | | T201 S350 S381 | | bacterial MoeB/ThiF/HesA family and | |
| | | | T386 S393 T134 | | ubiquitin-activating enzyme E1: R72- | |
| | | | T243 S296 | | P221 | |
| | | | | | Molybdopterin biosynthesis (MOEB | BLAST-PRODOM |
| | | | | | proteins): PD035666: G222-I368 | |
| | | | | | Ubiquitin-activating enzyme: | BLAST-DOMO |
| | | | | | DM00412\|A49890\|1-238: Y53-P232 | |
| 2 | 2116390CD1 | 242 | Y57 Y216 S13 | | Class II Aldolase and Adducin N- | HMMER-PFAM |
| | | | S49 S89 T106 | | terminal domain Aldolase II: R25-V242 | |
| | | | S223 T148 S2 | | Class II Aldolase and Adducin N- | BLIMPS-PFAM |
| | | | T136 T167 T209 | | terminal domain PF00596: T44-L50, I56- | |
| | | | | | D77, K93-S118, V190-A207 | |
| | | | | | PROTEIN ISOMERASE ZINC PROBABLE SUGAR | BLAST-PRODOM |
| | | | | | ADDUCIN CYTOSKELETON MEMBRANE | |
| | | | | | CALMODULIN-BINDING ALTERNATIVE 1390: | |
| | | | | | E30-A207 | |
| 3 | 5785224CD1 | 404 | T155 S136 T192 | | PROTEIN RACEMASE BILE ACID-INDUCIBLE | BLAST-PRODOM |
| | | | S293 T301 | | OPERON F ISOMERASE TRANSFERASE | |
| | | | | | ALPHAMETHYLACYL COA L-CARNITINE | |
| | | | | | PD002990: P6-T127 | |
| | | | | | TRANSFERASE OF SECTION PROTEIN FORMYL | |
| | | | | | COA PD150845: K262-E380 | |
| | | | | | PROTEIN RACEMASE ISOMERASE BILE ACID- | |
| | | | | | INDUCIBLE OPERON F TRANSFERASE | |
| | | | | | ALPHAMETHYLACYL COA L-CARNITINE | |
| | | | | | PD003472: G128-C261 | |
| 4 | 1870996CD1 | 126 | T57 S113 | | Signal peptide: M1-A59 | SPSCAN |
| | | | | | PROTEIN CELL DIVISION OF INHIBITOR | BLAST-PRODOM |
| | | | | | SECTION YFHF SULA PD011289: M1-V125 | |
| | | | | | CELL DIVISION INHIBITOR | BLAST-DOMO |
| | | | | | DM05984\|A64110\|1-296: L5-V125 | |
| 5 | 138841CD1 | 384 | T147 T194 S324 | N206 | Carbon-nitrogen hydrolase: I71-Y365 | HMMER-PFAM |
| | | | T359 S8 S48 | | | |
| | | | S141 T299 Y57 | | Nitrilases/Cyanide hydrolase: R80- | BLIMPS-BLOCKS |
| | | | Y312 | | A126, H198-I252 | |
| | | | | | Nitrilase aliphatic amidase: V89-A258 | BLAST-PRODOM |
| | | | | | N-CARBAMOYL AMIDO HYDROLASE: M1-I71 | |
| 6 | 1485405CD1 | 164 | T141 S43 T71 | | Asparaginase: M1-K164 | HMMER-PFAM |
| | | | S80 | | HYDROLASE N4- PRECURSOR: I81-A126 | BLIMPS_PRODOM |
| | | | | | Precursor hydrolase signal: V5-H114 | BLAST-PRODOM |
| | | | | | GLYCOSYL ASPARAGINASE CHAIN: L38-K152 | BLAST-DOMO |
| 7 | 2024617CD1 | 602 | S3 T50 S313 | N308 N484 | Ankyrin repeat: D518-R551, W552-Q585 | HMMER-PFAM |
| | | | S444 T79 S189 | | Ankyrin repeat proteins: L523-L538, | BLIMPS_PFAM |
| | | | S255 S313 S508 | | G553-Q562 | |
| | | | T580 T584 Y78 | | Hydrolase glutaminase: N283-L490 | BLAST-PRODOM |
| | | | Y322 | | Glutaminase isoform; liver; kidney: M68- | BLAST-DOMO |
| | | | | | L502 | |
| | | | | | Ankyrin repeat: R504-E540 | |
| 8 | 4721827CD1 | 434 | S68 T344 S31 | N2 N186 N336 | Glycosyl Hydrolase Family: D417-L423 | BLIMPS-PRINTS |
| | | | T47 S51 S68 S79 | N346 | N-ACETYLGLUCOSAMINE-6-SULFATASE | BLAST-PRODOM |
| | | | S83 T93 T174 | | PRECURSOR: C329-G400 | |
| | | | T178 S198 Y208 | | | |
| 9 | 5406614CD1 | 369 | S200 S222 S224 | | Exonuclease: I70-G78, V126-A145 | BLIMPS_PFAM |
| | | | S245 S318 T366 | | | |
| | | | T283 S293 T311 | | | |
| 10 | 1252792CD1 | 453 | T114 S203 S328 | N135 N273 | ATP/GTP binding site motif (P loop): | MOTIFS |
| | | | T391 S408 T54 | N314 N353 | G162-K168 | |
| | | | T114 S120 S166 | | Signal cleavage: M1-A20 | SPSCAN |
| | | | S177 S366 S407 | | | |
| | | | S408 | | | |
| 11 | 1299305CD1 | 511 | T436 S74 T231 | N495 | Leucine zipper: L365-L386 | MOTIFS |
| | | | T323 T367 Y200 | | Beta-lactamases classes-A, -C, and-D | PROFILESCAN |
| | | | T9 S74 T173 | | active site: G106-V153 | |
| | | | S268 S356 S439 | | PROTEIN PRECURSOR HYDROLASE: E73-A211 | BLAST-PRODOM |
| 12 | 1385190CD1 | 520 | S510 S180 T215 | N30 N251 | ATP/GTP binding site motif (P loop): | MOTIFS |
| | | | T491 S117 S193 | | G211-K217 | |
| | | | T228 S254 S277 | | | |
| | | | T298 Y299 S355 | | | |
| | | | S436 | | | |
| 13 | 2133162CD1 | 687 | S257 T632 S2 | N88 N173 N283 | ATP/GTP binding site motif (P loop): | MOTIFS |
| | | | T242 T252 S256 | | A615-K621 | |
| | | | T383 T402 T422 | | | |
| | | | S431 S527 T547 | | | |
| | | | S595 S608 T622 | | | |
| | | | T641 S667 S40 | | | |
| | | | T90 S117 S232 | | | |
| | | | S234 S236 S501 | | | |
| | | | S503 S518 S519 | | | |
| | | | T552 S585 | | | |
| 14 | 254567CD1 | 190 | S187 S188 T86 | N168 | ATP/GTP binding site motif (P loop): | MOTIFS |
| | | | | | G9-T16 | |
| | | | | | CBXX/CFQX SUPERFAMILY SIGNATURE: H4-I19 | BLIMPS-PRINTS |
| | | | | | ATP-BINDING PROTEIN: V5-Q186 | BLAST-PRODOM |
| 15 | 3903488CD1 | 284 | Y196 S14 S68 | N31 N47 | ATP/GTP binding site motif (P loop): | MOTIFS |
| | | | S148 S180 S208 | | G10-S17 | |
| | | | T215 T174 S197 | | Signal cleavage: M1-A29 | SPSCAN |
| | | | S202 S236 S272 | | ATP-BINDING PROTEIN: L7-E269 | BLAST-PRODOM |
| | | | | | ATP NP BIND: M1-D268 | BLAST_DOMO |
| 16 | 5391816CD1 | 245 | T191 Y165 S37 | | Epoxide hydrolase signature: I128-L141 | BLIMPS-PRINTS |
| | | | S223 | | | |
| 17 | 5886989CD1 | 231 | S12 T99 T120 | | ATP/GTP binding site motif (P loop): | MOTIFS |
| | | | T166 T185 T87 | | G8-K14 | |
| | | | T194 S149 | | Signal cleavage: M1-G13 | SPSCAN |
| | | | | | Uridine kinase signature: M1-I18 | BLIMPS-PRINTS |
| | | | | | ATP-BINDING 3′REGION: F2-G180 | BLAST-PRODOM |
| | | | | | ATP NP BIND: M1-G180 | BLAST-DOMO |
| 18 | 723432CD1 | 475 | T117 S130 T150 | | INTERGENIC REGION CONTAINING TRPASP | BLAST-PRODOM |
| | | | S168 S223 S235 | | REPEATS: N157-Y456 | |
| | | | S249 T270 S370 | | A Beta-transducin family trp-asp | BLAST-DOMO |
| | | | T423 S439 S446 | | repeats: A3-Q472 | |
| | | | Y377 S223 S299 | | | |
| | | | S341 T362 T428 | | | |
| | | | T183 | | | |
| 19 | 1332963CD1 | 421 | S41 T131 S132 | N153 N269 | Signal peptide: M1-A20 | SIGPEPT |
| | | | S169 S246 S406 | | PROTEIN TRANSFERASE: R130-K215 | BLAST-PRODOM |
| | | | S410 S169 T255 | | | |
| | | | S284 T307 S380 | | | |
| | | | Y58 S218 | | | |
| 20 | 1559410CD1 | 310 | S178 S20 | N300 | ATP/GTP binding site motif (P loop): | MOTIFS |
| | | | | | G16-T23 | |
| | | | | | Leucine zipper: L81-L102 | |
| | | | | | Adenylate kinase signature: A13-C26 | BLIMPS-PRINTS |
| | | | | | ATP-BINDING HAM1-PEM2 INTERGENIC | BLAST-PRODOM |
| | | | | | REGION: I15-L182 | |
| | | | | | ATP NP BIND: G11-R269 | BLAST-DOMO |
| 21 | 1752587CD1 | 194 | S25 T87 | | Signal peptide: M1-W20 | SIGPEPT |
| | | | | | Signal cleavage: M1-G17 | SPSCAN |
| | | | | | Transmembrane domain: M4-D23 | TMAP |
| 22 | 1911509CD1 | 349 | S122 S134 S238 | N303 N71 N265 | Abeta hydrolase: R102-C345 Alpha/beta | HMMER-PFAM |
| | | | T244 S298 T49 | | hydrolase fold | |
| | | | T65 S73 T100 | | Alpha/beta hydrolase fold signature: | BLIMPS-PRINTS |
| | | | S115 S163 S310 | | N101-S116, L149-162, S163-V176 | |
| | | | S237 | | HYDROLASE: K52-F181 | BLAST-PRODOM |
| | | | | | Hydrolase: H70-P182 | BLAST-DOMO |
| 23 | 2210170CD1 | 245 | S14 T44 S74 S97 | N95 | ATP/GTP binding site motif (P loop): | MOTIFS |
| | | | T238 S163 T209 | | G75-K81 | |
| | | | | | Signal peptide: M1-A23 | SIGPEPT |
| | | | | | Mrp (ATP-binding) family proteins: V66- | BLIMPS-BLOCKS |
| | | | | | L92, I101-V113, V154-V195 | |
| | | | | | HYPOTHETICAL ATP-BINDING PROTEIN: I165- | BLAST-PRODOM |
| | | | | | V234 | |
| | | | | | NIFH/FRXC FAMILY: V66-M224 | BLAST-DOMO |
| 24 | 234664CD1 | 255 | S47 S194 T85 | N83 | Hydrogen uptake protein: I62-Y74 | BLIMPS-PRINTS |
| | | | S244 | | | |
| | | | | | ENDOTHELIN-CONVERTING HYDROLASE: M97- | BLAST-PRODOM |
| | | | | | E160 | |
| 25 | 2884114CD1 | 377 | S26 S43 S63 | N136 | Histone deacetylase family: V17-G324 | HMMER-PFAM |
| | | | Y174 T192 T311 | | | |
| | | | S351 S39 | | Histone deacetylase family: E65-L79, | BLIMPS-PFAM |
| | | | | | D101-G117, G139-I162, R171-M196, F208- | |
| | | | | | Y240, I243-I284, I300-R313 | |
| | | | | | HISTONE DEACETYLASE: R37-G324 | BLAST-PRODOM |
| | | | | | RPD3 histone deacetylase/ACUC acetoin | BLAST-DOMO |
| | | | | | utilization: P35-V321 | |
| 26 | 4103559CD1 | 403 | T7 T108 S215 | N113 N316 | ATP/GTP binding site motif (P loop): | MOTIFS |
| | | | T297 S330 S359 | | G293-K299 | |
| | | | S372 S93 S94 | | AAA:Atpases associated with various | HMMER-PFAM |
| | | | T155 S173 S365 | | cellular activities: G288-W403 | |
| | | | Y343 S174 | | ENDOPEPTIDASE LA (LON) S: G293-T312 | BLIMPS-PRINTS |
| | | | | | ATP-dependent CLP protease ATP-binding | |
| | | | | | subunit: L289-A307 | |
| | | | | | AAA-protein family protein: W252-P272, | BLIMPS-BLOCKS |
| | | | | | W186-A307, A319-R361 | |
| | | | | | ATP-BINDING PROTEASE SUBUNIT HOMOLOG: | BLAST-PRODOM |
| | | | | | T300-P402 | |
| | | | | | AAA-PROTEIN FAMILY: P248-W403 | BLAST-DOMO |

TABLE 4

| | Incyte | | | | | |
|---|---|---|---|---|---|---|
| Polynucleotide | Polynucleotide | Sequence | Selected | | 5′ | 3′ |
| SEQ ID NO: | ID | Length | Fragment(s) | Sequence Fragments | Position | Position |
| 27 | 2783442CB1 | 1478 | 1-537 | 3540084H1 (SEMVNOT04) | 1 | 290 |
| | | | | 1619664T6 (BRAITUT13) | 807 | 1478 |
| | | | | 2084618R6 (PANCNOT04) | 255 | 882 |
| | | | | 897793R1 (BRSTNOT05) | 531 | 1142 |
| 28 | 2116390CB1 | 1183 | 1-62 | 190070F1 (SYNORAB01) | 404 | 1183 |
| | | | | 776806R1 (COLNNOT05) | 1 | 565 |
| 29 | 5785224CB1 | 1572 | 1-656 | 644802R6 (BRSTTUT02) | 401 | 855 |
| | | | | 5785224H1 (FIBRTXS07) | 699 | 994 |
| | | | | 2506827F6 (CONUTUT01) | 1 | 516 |
| | | | | 1212335T6 (BRSTTUT01) | 929 | 1572 |
| 30 | 1870996CB1 | 1335 | 1-170, 255-332 | 1795181R6 (PROSTUT05) | 1331 | 1335 |
| | | | | 1398049F1 (BRAITUT08) | 802 | 1335 |
| | | | | SXAD90083V1 | 1 | 463 |
| | | | | 2182959H1 (SININOT01) | 378 | 770 |
| | | | | SXAF00571V1 | 651 | 1335 |
| | | | | 3507430H1 (CONCNOT01) | 464 | 790 |
| 31 | 138841CB1 | 1341 | 1-46 | 138841R6 (LIVRNOT01) | 1 | 480 |
| | | | | 167939R6 (LIVRNOT01) | 820 | 1341 |
| | | | | SCFA05578V1 | 515 | 1094 |
| | | | | SCFA04593D1 | 392 | 1061 |
| 32 | 1485405CB1 | 755 | | 831524H1 (PROSTUT04) | 538 | 755 |
| | | | | 1971451F6 (UCMCL5T01) | 370 | 747 |
| | | | | 3075952H1 (BONEUNT01) | 1 | 292 |
| | | | | 1970651X301D1 (UCMCL5T01) | 213 | 684 |
| 33 | 2024617CB1 | 2390 | 1-1688 | 1444713T6 (THYRNOT03) | 1785 | 2368 |
| | | | | g2795888 | 1619 | 2375 |
| | | | | 4144157F6 (BRSTTMT01) | 983 | 1524 |
| | | | | g872924 | 1948 | 2390 |
| | | | | 2026172X303D1 (KERANOT02) | 705 | 1218 |
| | | | | 2024617X304B1 (KERANOT02) | 1794 | 2372 |
| | | | | 2024617X302D1 (KERANOT02) | 48 | 567 |
| | | | | 2026172X308D1 (KERANOT02) | 1 | 481 |
| | | | | 2024617R6 (KERANOT02) | 1356 | 1770 |
| | | | | 1444713T2 (THYRNOT03) | 1746 | 2364 |
| | | | | g3431607 | 1942 | 2378 |
| 34 | 4721827CB1 | 2940 | 1-265, 856-2240 | 1730047T6 (BRSTTUT08) | 1747 | 2273 |
| | | | | 5202590T6 (STOMNOT08) | 200 | 835 |
| | | | | 1730047F6 (BRSTTUT08) | 777 | 1222 |
| | | | | 4935430H1 (BRSTTUT20) | 942 | 1231 |
| | | | | 827193H1 (PROSNOT06) | 574 | 846 |
| | | | | 2107214R6 (BRAITUT03) | 2558 | 2940 |
| | | | | 1819064F6 (PROSNOT20) | 1238 | 1703 |
| | | | | 2844607T6 (DRGLNOT01) | 1686 | 2268 |
| | | | | 1538938T1 (SINTTUT01) | 2257 | 2916 |
| | | | | 5515657H1 (LIVRDIR01) | 1 | 257 |
| | | | | 2844607F6 (DRGLNOT01) | 1174 | 1686 |
| 35 | 5406614CB1 | 1417 | 1-840 | 1636138F6 (UTRSNOT06) | 930 | 1417 |
| | | | | 1291844F1 (PGANNOT03) | 265 | 863 |
| | | | | 875716R1 (LUNGAST01) | 557 | 1173 |
| | | | | SBVA00533V1 | 1 | 455 |
| 36 | 1252792CB1 | 2133 | 1-863, | 1252792F6 (LUNGFET03) | 1599 | 2133 |
| | | | 2111-2133 | 618039R6 (PGANNOT01) | 854 | 1401 |
| | | | | 1255536F2 (MENITUT03) | 210 | 769 |
| | | | | 1571325F6 (UTRSNOT05) | 1017 | 1624 |
| | | | | 2586114H1 (BRAITUT22) | 1 | 243 |
| | | | | 2688507H1 (LUNGNOT23) | 718 | 961 |
| 37 | 1299305CB1 | 1829 | 1-750 | 1299305F6 (BRSTNOT07) | 783 | 1306 |
| | | | | SXYA00951V1 | 249 | 772 |
| | | | | 1299305X349V1 (BRSTNOT07) | 1297 | 1828 |
| | | | | 218730F1 (STOMNOT01) | 1256 | 1826 |
| | | | | 1299305X352V1 (BRSTNOT07) | 1309 | 1829 |
| | | | | SXYA02187V1 | 624 | 1046 |
| | | | | SXYA01827V1 | 298 | 798 |
| | | | | 3692261H1 (HEAANOT01) | 1 | 301 |
| 38 | 1385190CB1 | 2323 | 1-1607 | 4560082H1 (KERATXT01) | 605 | 847 |
| | | | | 1464459R1 (PANCNOT04) | 771 | 1300 |
| | | | | 866833R1 (BRAITUT03) | 1746 | 2323 |
| | | | | 817831H1 (OVARTUT01) | 492 | 737 |
| | | | | 4621649H1 (ENDVNOT01) | 1421 | 1691 |
| | | | | 1436421F1 (PANCNOT08) | 1280 | 1645 |
| | | | | 140593F1 (TLYMNOR01) | 1688 | 2310 |
| | | | | 4384773H1 (BRAVUTT02) | 1 | 247 |
| | | | | 476121R6 (MMLR2DT01) | 107 | 614 |
| | | | | 2124443T6 (BRSTNOT07) | 1660 | 2308 |
| 39 | 2133162CB1 | 2992 | 1-107, 901-1515, | 2622173H1 (KERANOT02) | 2267 | 2543 |
| | | | 2398-2441 | 3605342H1 (LUNGNOT30) | 727 | 1042 |
| | | | | 040203R6 (TBLYNOT01) | 2018 | 2480 |
| | | | | 1573212F6 (LNODNOT03) | 839 | 1282 |
| | | | | 1829765F6 (THP1AZT01) | 2448 | 2981 |
| | | | | 960713H1 (BRSTTUT03) | 2751 | 2992 |
| | | | | 2539058F6 (BONRTUT01) | 1 | 766 |
| | | | | 2174569F6 (ENDCNOT03) | 1216 | 1726 |
| | | | | SASA00952F1 | 1378 | 1897 |
| | | | | 1573212T6 (LNODNOT03) | 1733 | 2260 |
| | | | | 3878345H1 (SPLNNOT11) | 1979 | 2268 |
| 40 | 254567CB1 | 850 | | 3142967H1 (HNT2AZS07) | 1 | 312 |
| | | | | 2192041H1 (THYRTUT03) | 595 | 850 |
| | | | | 1397586T1 (BRAITUT08) | 405 | 847 |
| | | | | 1859156T6 (PROSNOT18) | 190 | 835 |
| 41 | 3903488CB1 | 1472 | 1-58, 1387-1450 | 3240016H1 (COLAUCT01) | 1336 | 1472 |
| | | | | 1353041T6 (LATRTUT02) | 480 | 1019 |
| | | | | 1216572R1 (BRSTTUT01) | 1 | 586 |
| | | | | 2286017T6 (BRAINON01) | 675 | 1375 |
| 42 | 5391816CB1 | 1561 | | 827147R1 (PROSNOT06) | 988 | 1561 |
| | | | | 1283691F6 (COLNNOT16) | 904 | 1522 |
| | | | | 118023R1 (MUSCNOT01) | 416 | 1342 |
| | | | | 2608576F6 (BONTNOT01) | 1 | 516 |
| 43 | 5886989CB1 | 1949 | 566-754, 1-245 | 1963212R6 (BRSTNOT04) | 1319 | 1749 |
| | | | | 1504160F1 (BRAITUT07) | 541 | 1107 |
| | | | | 931121H1 (CERVNOT01) | 1714 | 1949 |
| | | | | SBRA05080D1 | 655 | 1287 |
| | | | | 3335260F6 (BRAIFET01) | 1 | 646 |
| | | | | 870085R6 (LUNGAST01) | 1128 | 1726 |
| 44 | 723432CB1 | 2221 | 28-117 | 1985045R6 (LUNGAST01) | 1629 | 2221 |
| | | | | 723432R1 (SYNOOAT01) | 816 | 1394 |
| | | | | 5637189H1 (UTRSTMR01) | 1 | 262 |
| | | | | 1426405X13F1 (SINTBST01) | 275 | 842 |
| | | | | 2072529X11C1 (ISLTNOT01) | 142 | 687 |
| | | | | SAFC01647F1 | 1344 | 1917 |
| | | | | SAFC01486F1 | 885 | 1418 |
| 45 | 1332963CB1 | 1619 | 1-24, 735-1025 | SBMA02284F1 | 255 | 924 |
| | | | | 859640R1 (BRAITUT03) | 553 | 1133 |
| | | | | 2955002F6 (KIDNFET01) | 1 | 518 |
| | | | | 1576265T1 (LNODNOT03) | 981 | 1619 |
| 46 | 1559410CB1 | 1448 | 1-227 | 1811269F6 (PROSTUT12) | 617 | 1147 |
| | | | | 4637615H1 (MYEPTXT01) | 500 | 740 |
| | | | | 769274H1 (COLNCRT01) | 1333 | 1448 |
| | | | | 1798960F6 (COLNNOT27) | 960 | 1447 |
| | | | | 3520838H1 (LUNGNON03) | 33 | 364 |
| | | | | 4062190H1 (BRAINOT21) | 358 | 529 |
| | | | | 3593730H1 (FIBPNOT01) | 1 | 207 |
| 47 | 1752587CB1 | 2225 | 1-44, 562-1515 | SCMA05260V1 | 870 | 1460 |
| | | | | 098895H1 (PITUNOR01) | 1 | 234 |
| | | | | 1500773F6 (SINTBST01) | 1665 | 2225 |
| | | | | SCMA05704V1 | 237 | 719 |
| | | | | SCMA00926V1 | 1317 | 1802 |
| | | | | 4864824T6 (PROSTUT09) | 1575 | 2203 |
| | | | | SCMA03952V1 | 45 | 497 |
| | | | | SCMA04987V1 | 607 | 1211 |
| 48 | 1911509CB1 | 1400 | 1-28 | 2148490F6 (LUNGNOT18) | 448 | 919 |
| | | | | 1404974F6 (LATRTUT02) | 1197 | 1400 |
| | | | | 1963768T6 (BRSTNOT04) | 726 | 1384 |
| | | | | 2149013H1 (BRAINOT09) | 1131 | 1393 |
| | | | | 4897260H1 (LIVRTUT12) | 380 | 689 |
| | | | | 1911509F6 (CONNTUT01) | 1 | 445 |
| 49 | 2210170CB1 | 1361 | 739-1103 | SBUA01233D1 | 342 | 840 |
| | | | | 2210170F6 (SINTFET03) | 1 | 372 |
| | | | | SBUA00454D1 | 500 | 993 |
| | | | | 2210170T6 (SINTFET03) | 895 | 1361 |
| 50 | 234664CB1 | 993 | 1-34 | SXCA00325V1 | 1 | 497 |
| | | | | SCLA05768V1 | 611 | 993 |
| | | | | SCLA00227V1 | 525 | 984 |
| | | | | 2172996T6 (ENDCNOT03) | 433 | 979 |
| 51 | 2884114CB1 | 1367 | 774-1367 | 2047322F6 (THP1T7T01) | 166 | 652 |
| | | | | 4288169X302D1 (LIVRDIR01) | 1 | 381 |
| | | | | 1446241F6 (PLACNOT02) | 979 | 1367 |
| | | | | 5616107H1 (THYMNOR02) | 949 | 1268 |
| | | | | 4794983H1 (LIVRTUT09) | 611 | 870 |
| | | | | 4288169H1 (LIVRDIR01) | 800 | 1108 |
| 52 | 4103559CB1 | 1317 | 1-33, 76-355 | g1894065 | 356 | 735 |
| | | | | 3356420H1 (PROSTUT16) | 1 | 270 |
| | | | | 3356420T6 (PROSTUT16) | 587 | 1274 |
| | | | | g2784609 | 868 | 1317 |
| | | | | 3356420F6 (PROSTUT16) | 2 | 555 |

TABLE 5

| Polynucleotide | Incyte | |
|---|---|---|
| SEQ ID NO: | Project ID | Representative Library |
| 27 | 2783442CB1 | PANCTUT02 |
| 28 | 2116390CB1 | BLADTUT04 |
| 29 | 5785224CB1 | CONUTUT01 |
| 30 | 1870996C31 | PROSTUT05 |
| 31 | 138841CB1 | LIVRNOT01 |
| 32 | 1485405CB1 | UCMCL5T01 |
| 33 | 2024617CB2 | LIVRTUT04 |
| 34 | 4721827CB1 | BRSTTUT08 |
| 35 | 5406614CB1 | LUNGAST01 |
| 36 | 1252792CB1 | BRAITUT01 |
| 37 | 1299305CB1 | MMLR1DT01 |
| 38 | 1385190CB1 | LUNGAST01 |
| 39 | 2133162CB1 | TLYMNOT06 |
| 40 | 254567CB1 | PROSNOT13 |
| 41 | 3903488CB1 | LUNGNOT09 |
| 42 | 5391816CB2 | LUNGFET03 |
| 43 | 5886989CB1 | CORPNOT02 |
| 44 | 723432CB1 | BRAITUT02 |
| 45 | 1332963CB1 | BRAITUT03 |
| 46 | 15594100B1 | PROSTUT12 |
| 47 | 1752587CB1 | SINTBST01 |
| 48 | 1911509CB1 | SINTNOT02 |
| 49 | 2210170CB2 | SINTFET03 |
| 50 | 234664CB1 | KIDNTUT13 |
| 51 | 2884114CB1 | THP1T7T01 |
| 52 | 4103559CB1 | PROSTUT16 |

TABLE 6

| Library | Vector | Library Description |
|---|---|---|
| BLADTUT04 | pINCY | Library was constructed using RNA isolated from bladder tumor tissue removed from a |
| | | 60-year-old Caucasian male during a radical cystectomy, prostatectomy, and |
| | | vasectomy. Pathology indicated grade 3 transitional cell carcinoma in the left |
| | | bladder wall. Carcinoma in-situ was identified in the dome and trigone. Patient |
| | | history included tobacco use. Family history included type I diabetes, malignant |
| | | neoplasm of the stomach, atherosclerotic coronary artery disease, and acute |
| | | myocardial infarction. |
| BRAITUT01 | PSPORT1 | Library was constructed using RNA isolated from brain tumor tissue removed from a |
| | | 50-year-old Caucasian female during a frontal lobectomy. Pathology indicated |
| | | recurrent grade 3 oligoastrocytoma with focal necrosis and extensive calcification. |
| | | Patient history included a speech disturbance and epilepsy. The patient's brain |
| | | had also been irradiated with a total dose of 5,082 cyg (Fraction 8). Family |
| | | history included a brain tumor. |
| BRAITUT02 | PSPORT1 | Library was constructed using RNA isolated from brain tumor tissue removed from the |
| | | frontal lobe of a 58-year-old Caucasian male during excision of a cerebral |
| | | meningeal lesion. Pathology indicated a grade 2 metastatic hypernephroma. Patient |
| | | history included a grade 2 renal cell carcinoma, insomnia, and chronic airway |
| | | obstruction. Family history included a malignant neoplasm of the kidney. |
| BRAITUT03 | PSPORT1 | Library was constructed using RNA isolated from brain tumor tissue removed from the |
| | | left frontal lobe of a 17-year-old Caucasian female during excision of a cerebral |
| | | meningeal lesion. Pathology indicated a grade 4 fibrillary giant and small-cell |
| | | astrocytoma. Family history included benign hypertension and cerebrovascular |
| | | disease. |
| BRSTTUT08 | pINCY | Library was constructed using RNA isolated from breast tumor tissue removed from a |
| | | 45-year-old Caucasian female during unilateral extended simple mastectomy. |
| | | Pathology indicated invasive nuclear grade 2-3 adenocarcinoma, ductal type, with 3 |
| | | of 23 lymph nodes postive for metastatic disease. Greater than 50% of the tumor |
| | | volume was in situ, both comedo and non-comedo types. Immunostains were positive |
| | | for estrogen/progesterone receptors, and uninvolved tissue showed proliferative |
| | | changes. The patient concurrently underwent a total abdominal hysterectomy. |
| | | Patient history included valvuloplasty of mitral valve without replacement, |
| | | rheumatic mitral insufficiency, and rheumatic heart disease. Family history |
| | | included acute myocardial infarction, atherosclerotic coronary artery disease, and |
| | | type II diabetes. |
| CONUTUT01 | pINCY | Library was constructed using RNA isolated from sigmoid mesentery tumor tissue |
| | | obtained from a 61-year-old female during a total abdominal hysterectomy and |
| | | bilateral salpingo-oophorectomy with regional lymph node excision. Pathology |
| | | indicated a metastatic grade 4 malignant mixed mullerian tumor present in the |
| | | sigmoid mesentery at two sites. |
| CORPNOT02 | pINCY | Library was constructed using RNA isolated from diseased corpus callosum tissue |
| | | removed from the brain of a 74-year-old Caucasian male who died from Alzheimer's |
| | | disease. |
| KIDNTUT13 | pINCY | Library was constructed using RNA isolated from kidney tumor tissue removed from a |
| | | 51-year-old Caucasian female during a nephroureterectomy. Pathology indicated a |
| | | grade 3 renal cell carcinoma. Patient history included depressive disorder, |
| | | hypoglycemia, and uterine endometriosis. Family history included calculus of the |
| | | kidney, colon cancer, and type II diabetes. |
| LIVRNOT01 | PBLUESCRIPT | Library was constructed at Stratagene, using RNA isolated from the liver tissue of |
| | | a 49-year-old male. |
| LIVRTUT04 | pINCY | Library was constructed using RNA isolated from liver tumor tissue removed from a |
| | | 50-year-old Caucasian male during a partial hepatectomy. Pathology indicated a |
| | | grade 3-4 hepatoma, forming a mass. Patient history included benign hypertension |
| | | and hepatitis. Hepatitis B core antigen and hepatitis B surface antigen was present |
| | | in the patient. |
| LUNGAST01 | PSPORT1 | Library was constructed using RNA isolated from the lung tissue of a 17-year-old |
| | | Caucasian male, who died from head trauma. Patient history included asthma. |
| LUNGFET03 | pINCY | Library was constructed using RNA isolated from lung tissue removed from a |
| | | Caucasian female fetus, who died at 20 weeks' gestation. |
| LUNGNOT09 | pINCY | Library was constructed using RNA isolated from the lung tissue of a 23-week-old |
| | | Caucasian male fetus. The pregnancy was terminated following a diagnosis by |
| | | ultrasound of infantile polycystic kidney disease. |
| MMLR1DT01 | PSPORT1 | Library was constructed using RNA isolated from adherent mononuclear cells, which |
| | | came from a pool of male and female donors. The cells were cultured for 24 hours |
| | | following Ficoll Hypaque centrifugation. |
| PANCTUT02 | pINCY | Library was constructed using RNA isolated from pancreatic tumor tissue removed |
| | | from a 45-year-old Caucasian female during radical pancreaticoduodenectomy. |
| | | Pathology indicated a grade 4 anaplastic carcinoma. Family history included benign |
| | | hypertension, hyperlipidemia and atherosclerotic coronary artery disease. |
| PROSNOT18 | pINCY | Library was constructed using RNA isolated from diseased prostate tissue removed |
| | | from a 58-year-old Caucasian male during a radical cystectomy, radical |
| | | prostatectomy, and gastrostomy. Pathology indicated adenofibromatous hyperplasia; |
| | | this tissue was associated with a grade 3 transitional cell carcinoma. Patient |
| | | history included angina and emphysema. Family history included acute myocardial |
| | | infarction, atherosclerotic coronary artery disease, and type II diabetes. |
| PROSTUT05 | PSPORT1 | Library was constructed using RNA isolated from prostate tumor tissue removed from |
| | | a 69-year-old Caucasian male during a radical prostatectomy. Pathology indicated |
| | | adenocarcinoma (Gleason grade 3 + 4). Adenofibromatous hyperplasia was also present. |
| | | Family history included congestive heart failure, multiple myeloma, hyperlipidemia, |
| | | and rheumatoid arthritis. |
| PROSTUT12 | pINCY | Library was constructed using RNA isolated from prostate tumor tissue removed from |
| | | a 65-year-old Caucasian male during a radical prostatectomy. Pathology indicated |
| | | an adenocarcinoma (Gleason grade 2 + 2). Adenofibromatous hyperplasia was also |
| | | present. The patient presented with elevated prostate specific antigen (PSA). |
| PROSTUT16 | pINCY | Library was constructed using RNA isolated from prostate tumor tissue removed from |
| | | a 55-year-old Caucasian male. Pathology indicated adenocarcinoma, Gleason grade |
| | | 5 + 4. Adenofibromatous hyperplasia was also present. The patient presented with |
| | | elevated prostate specific antigen (PSA) . Patient history included calculus of the |
| | | kidney. Family history included lung cancer and breast cancer. |
| SINTBST01 | pINCY | Library was constructed using RNA isolated from ileum tissue obtained from an 18- |
| | | year-old Caucasian female during bowel anastomosis. Pathology indicated Crohn's |
| | | disease of the ileum, involving 15 cm of the small bowel. Family history included |
| | | cerebrovascular disease and atherosclerotic coronary artery disease. |
| SINTFET03 | pINCY | Library was constructed using RNA isolated from small intestine tissue removed from |
| | | a Caucasian female fetus, who died at 20 weeks' gestation. |
| SINTNOT02 | PBLUESCRIPT | Library was constructed using RNA isolated from the small intestine of a 55-year- |
| | | old Caucasian female, who died from a subarachnoid hemorrhage. Serologies were |
| | | positive for cytomegalovirus (CMV). Previous surgeries included a hysterectomy. |
| THP1T7T01 | pINCY | Library was constructed using RNA isolated from 50,000 cultured THP-1 cells, which |
| | | was amplified using a proprietary T7 amplification method developed at Incyte. |
| | | THP-1 (ATCC TIB 202) is a human promonocyte line derived from the peripheral blood |
| | | of a 1-year-old caucasian male with acute monocytic leukemia (ref: Int. J. Cancer |
| | | (1980) 26:171). Antisense RNA (aRNA) was generated using T7 RNA polymerase |
| | | following the first round of cDNA synthesis. aRNA was then random-primed to |
| | | generate double-stranded cDNA, ligated to EcoR1 adaptors, and cloned non- |
| | | directionally into the pINCY vector (Incyte). The amplification technique used for |
| | | construction of this library does not significantly skew sequence abundance and can |
| | | be used for Transcript Imaging purposes. |
| TLYMNOT06 | pINCY | Library was constructed using 0.5 micrograms of polyA RNA isolated from activated |
| | | Th2 cells. These cells were differentiated from umbilical cord CD4 T cells with |
| | | IL-4 in the presence of anti-IL-12 antibodies and B7-transfected COS cells, and |
| | | then activated for six hours with anti-CD3 and anti-CD28 antibodies. cDNA |
| | | synthesis was initiated using a NotI-oligo(dT) primer. Double-stranded cDNA was |
| | | blunted, ligated to EcoRI adaptors, digested with NotI, size-selected, and cloned |
| | | into the NotI and EcoRI sites of the pINCY vector (Incyte). |
| UCMCL5T01 | PBLUESCRIPT | Library was constructed using RNA isolated from mononuclear cells obtained from the |
| | | umbilical cord blood of 12 individuals. The cells were cultured for 12 days with |
| | | IL-5 before RNA was obtained from the pooled lysates. |

TABLE 7

| Program | Description | Reference | Parameter Threshold |
|---|---|---|---|
| ABIFACTURA | A program that removes vector | Applied Biosystems, Foster City, CA. | |
| | sequences and masks ambiguous | | |
| | bases in nucleic acid sequences. | | |
| ABI/PARACELFDF | A Fast Data Finder useful in | Applied Biosystems, Foster City, CA; | Mismatch <50% |
| | comparing and annotating amino | Paracel Inc., Pasadena, CA. | |
| | acid or nucleic acid sequences. | | |
| ABI AutoAssembler | A program that assembles | Applied Biosystems, Foster City, CA. | |
| | nucleic acid sequences. | | |
| BLAST | A Basic Local Alignment Search | Altschul, S. F. et al. (1990) J. Mol. Biol. | ESTs: Probability value = 1.0E − 8 |
| | Tool useful in sequence simil- | 215:403-410; Altschul, S. F. et al. (1997) | or less |
| | arity search for amino acid and | Nucleic Acids Res. 25:3389-3402. | Full Length sequences: Probability |
| | nucleic acid sequences. BLAST | | value = 1.0E − 10 or less |
| | includes five functions: blastp, | | |
| | blastn, blastx, tblastn, and | | |
| | tblastx. | | |
| FASTA | A Pearson and Lipman | Pearson, W. R. and D. J. Lipman (1988) Proc. | ESTs: fasta E value = 1.06E − 6 |
| | algorithm that searches for | Natl. Acad Sci. USA 85:2444-2448; Pearson, | Assembled ESTs: fasta Identity = |
| | similarity between a query | W. R. (1990) Methods Enzymol. 183:63-98; | 95% or greater and |
| | sequence and a group of se- | and Smith, T. F. and M. S. Waterman (1981) | Match length = 200 bases or greater; |
| | quences of the same type. | Adv. Appl. Math. 2:482-489. | fastx E value = 1.0E− 8 or less |
| | FASTA comprises as least five | | Full Length sequences: |
| | functions: fasta, tfasta, fastx, | | fastx score = 100 or greater |
| | tfastx, and ssearch | | |
| BLIMPS | A BLocks IMProved Searcher | Henikoff, S. and J. G. Henikoff (1991) Nucleic | Probability value = 1.0E − 3 or less |
| | that matches a sequence against | Acids Res. 19:6565-6572; Henikoff, J. G. and | |
| | those in BLOCKS, PRINTS, | S. Henikoff (1996) Methods Enzymol. | |
| | DOMO, PRODOM, and PFAM | 266:88-105; and Attwood, T. K. et al. (1997) J. | |
| | databases to search for gene | Chem. Inf. Comput. Sci. 37:417-424. | |
| | families, sequence homology, | | |
| | and structural fingerprint | | |
| | regions. | | |
| HMMER | An algorithm for searching a | Krogh, A. et al. (1994) J. Mol. Biol. | PFAM hits: Probability value = |
| | query sequence against hidden | 235:1501-1531; Sonnhammer, E. L. L. et al. | 1.0E − 3 or less |
| | Markov model (HMM)-based | (1988) Nucleic Acids Res. 26:320-322; | Signal peptide hits: Score = 0 or |
| | databases of protein family | Durbin, R. et al. (1998) Our World View, in a | greater |
| | consensus sequences, such as | Nutshell, Cambridge Univ. Press, pp. 1-350. | |
| | PFAM. | | |
| ProfileScan | An algorithm that searches for | Gribskov, M. et al. (1988) CABIOS 4:61-66; | Normalized quality score ≧ GCG- |
| | structural and sequence motifs in | Gribskov, M. et al. (1989) Methods Enzymol. | specified “HIGH” value for that |
| | protein sequences that match | 183:146-159; Bairoch, A. et al. (1997) | particular Prosite motif. |
| | sequence patterns defined in | Nucleic Acids Res. 25:217-221. | Generally, score = 1.4 − 2.1. |
| | Prosite. | | |
| Phred | A base-calling algorithm that | Ewing, B. et al. (1998) Genome Res. | |
| | examines automated sequencer | 8:175-185; Ewing, B. and P. Green | |
| | traces with high sensitivity | (1998) GenomeRes. 8:186-194. | |
| | and probability. | | |
| Phrap | A Phils Revised Assembly | Smith, T. F. and M. S. Waterman (1981) Adv. | Score = 120 or greater; |
| | Program including SWAT and | Appl. Math. 2:482-489; Smith, T. F. and M. S. | Match length = 56 or greater |
| | CrossMatch, programs based on | Waterman (1981) J. Mol. Biol. 147:195-197; | |
| | efficient implementation of the | and Green, P., University of Washington, | |
| | Smith-Waterman algorithm, use- | Seattle, WA. | |
| | ful in searching sequence homo- | | |
| | logy and assembling DNA | | |
| | sequences. | | |
| Consed | A graphical tool for viewing and | Gordon, D. et al. (1998) Genome Res. 8:195-202. | |
| | editing Phrap assemblies. | | |
| SPScan | A weight matrix analysis | Nielson, H. et al. (1997) Protein Engineering | Score = 3.5 or greater |
| | program that scans protein | 10:1-6; Claverie, J. M. and S. Audic (1997) | |
| | sequences for the presence of | CABIOS 12:431-439. | |
| | secretory signal peptides. | | |
| TMAP | A program that uses weight | Persson, B. and P. Argos (1994) J. Mol. Biol. | |
| | matrices to delineate trans- | 237:182-192; Persson, B. and P. Argos (1996) | |
| | membrane segments on protein | Protein Sci. 5:363-371. | |
| | sequences and determine | | |
| | orientation. | | |
| TMHMMER | A program that uses a hidden | Sonnhammer, E. L. et al. (1998) Proc. Sixth Intl. | |
| | Markov model (HMM) to de- | Conf. on Intelligent Systems for Mol. Biol., | |
| | lineate transmembrane segments | Glasgow et al., eds., The Am. Assoc. for Artificial | |
| | on protein sequences and | Intelligence Press, Menlo Park, CA, pp. 175-182. | |
| | determine orientation. | | |
| Motifs | A program that searches amino | Bairoch, A. et al. (1997) Nucleic Acids Res. 25:217-221; | |
| | acid sequences for patterns that | Wisconsin Package Program Manual, version 9, page | |
| | matched those defined in | M51-59, Genetics Computer Group, Madison, WI. | |
| | Prosite. | | |

What is claimed is:

1. An isolated polypeptide comprising an amino acid sequence selected from the group consisting of:
a) an amino acid sequence selected from the group consisting of SEQ ID NO:1-26,
b) a naturally occurring amino acid sequence having at least 90% sequence identity to an amino acid sequence selected from the group consisting of SEQ ID NO:1-26,
c) a biologically active fragment of an amino acid sequence selected from the group consisting of SEQ ID NO:1-26, and
d) an immunogenic fragment of an amino acid sequence selected from the group consisting of SEQ ID NO:1-26.

2. An isolated polypeptide of claim 1 selected from the group consisting of SEQ ID NO:1-26.

3. An isolated polynucleotide encoding a polypeptide of claim 1.

4. An isolated polynucleotide encoding a polypeptide of claim 2.

5. An isolated polynucleotide of claim 4 selected from the group consisting of SEQ ID NO:27-52.

6. A recombinant polynucleotide comprising a promoter sequence operably linked to a polynucleotide of claim 3.

7. A cell transformed with a recombinant polynucleotide of claim 6.

8. A transgenic organism comprising a recombinant polynucleotide of claim 6.

9. A method for producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. An isolated antibody which specifically binds to a polypeptide of claim 1.

11. An isolated polynucleotide comprising a polynucleotide sequence selected from the group consisting of:
a) a polynucleotide sequence selected from the group consisting of SEQ ID NO:27-52,
b) a naturally occurring polynucleotide sequence having at least 90% sequence identity to a polynucleotide sequence selected from the group consisting of SEQ ID NO:27-52,
c) a polynucleotide sequence complementary to a),
d) a polynucleotide sequence complementary to b), and
e) an RNA equivalent of a)-d).

12. An isolated polynucleotide comprising at least 60 contiguous nucleotides of a polynucleotide of claim 11.

13. A method for detecting a target polynucleotide in a sample, said target polynucleotide having a sequence of a polynucleotide of claim 11, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

14. A method of claim 13, wherein the probe comprises at least 60 contiguous nucleotides.

15. A method for detecting a target polynucleotide in a sample, said target polynucleotide having a sequence of a polynucleotide of claim 11, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

16. A composition comprising an effective amount of a polypeptide of claim 1 and a pharmaceutically acceptable excipient.

17. A composition of claim 16, wherein the polypeptide comprises an amino acid sequence selected from the group consisting of SEQ ID NO:1-26.

18. A method for treating a disease or condition associated with decreased expression of functional HEM, comprising administering to a patient in need of such treatment the composition of claim 16.

19. A method for screening a compound for effectiveness as an agonist of a polypeptide of claim 1, the method comprising:
a) exposing a sample comprising a polypeptide of claim 1 to a compound, and
b) detecting agonist activity in the sample.

20. A composition comprising an agonist compound identified by a method of claim 19 and a pharmaceutically acceptable excipient.

21. A method for treating a disease or condition associated with decreased expression of functional HEM, comprising administering to a patient in need of such treatment a composition of claim 20.

22. A method for screening a compound for effectiveness as an antagonist of a polypeptide of claim 1, the method comprising:
a) exposing a sample comprising a polypeptide of claim 1 to a compound, and
b) detecting antagonist activity in the sample.

23. A composition comprising an antagonist compound identified by a method of claim 22 and a pharmaceutically acceptable excipient.

24. A method for treating a disease or condition associated with overexpression of functional HEM, comprising administering to a patient in need of such treatment a composition of claim 23.

25. A method of screening for a compound that specifically binds to the polypeptide of claim 1, said method comprising the steps of:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

26. A method of screening for a compound that modulates the activity of the polypeptide of claim 1, said method comprising:
a) combining the polypeptide of claim 1 with at least one test compound under conditions permissive for the activity of the polypeptide of claim 1,
b) assessing the activity of the polypeptide of claim 1 in the presence of the test compound, and
c) comparing the activity of the polypeptide of claim 1 in the presence of the test compound with the activity of the polypeptide of claim 1 in the absence of the test compound, wherein a change in the activity of the polypeptide of claim 1 in the presence of the test compound is indicative of a compound that modulates the activity of the polypeptide of claim 1.

27. A method for screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

28. A method for assessing toxicity of a test compound, said method comprising:
a) treating a biological sample containing nucleic acids with the test compound;
b) hybridizing the nucleic acids of the treated biological sample with a probe comprising at least 20 contiguous nucleotides of a polynucleotide of claim 11 under conditions whereby a specific hybridization complex is formed between said probe and a target polynucleotide in the biological sample, said target polynucleotide comprising a polynucleotide sequence of a polynucleotide of claim 11 or fragment thereof;
c) quantifying the amount of hybridization complex; and
d) comparing the amount of hybridization complex in the treated biological sample with the amount of hybridization complex in an untreated biological sample, wherein a difference in the amount of hybridization complex in the treated biological sample is indicative of toxicity of the test compound.

29. A method of claim 9, wherein the polypeptide has the sequence of SEQ ID NO:1.

30. A method of claim 9, wherein the polypeptide has the sequence of SEQ ID NO:2.

31. A method of claim 9, wherein the polypeptide has the sequence of SEQ ID NO:3.

32. A method of claim 9, wherein the polypeptide has the sequence of SEQ ID NO:4.

33. A method of claim 9, wherein the polypeptide has the sequence of SEQ ID NO:5.

34. A method of claim 9, wherein the polypeptide has the sequence of SEQ ID NO:6.

35. A method of claim 9, wherein the polypeptide has the sequence of SEQ ID NO:7.

36. A method of claim 9, wherein the polypeptide has the sequence of SEQ ID NO:8.

37. A method of claim 9, wherein the polypeptide has the sequence of SEQ ID NO:9.

38. A method of claim 9, wherein the polypeptide has the sequence of SEQ ID NO:10.

39. A method of claim 9, wherein the polypeptide has the sequence of SEQ ID NO:11.

40. A method of claim 9, wherein the polypeptide has the sequence of SEQ ID NO:12.

41. A method of claim 9, wherein the polypeptide has the sequence of SEQ ID NO:13.

42. A method of claim 9, wherein the polypeptide has the sequence of SEQ ID NO:14.

43. A method of claim 9, wherein the polypeptide has the sequence of SEQ ID NO:15.

44. A method of claim 9, wherein the polypeptide has the sequence of SEQ ID NO:16.

45. A method of claim 9, wherein the polypeptide has the sequence of SEQ ID NO:17.

46. A method of claim 9, wherein the polypeptide has the sequence of SEQ ID NO:18.

47. A method of claim 9, wherein the polypeptide has the sequence of SEQ ID NO:19.

48. A method of claim 9, wherein the polypeptide has the sequence of SEQ ID NO:20.

49. A method of claim 9, wherein the polypeptide has the sequence of SEQ ID NO:21.

50. A method of claim 9, wherein the polypeptide has the sequence of SEQ ID NO:22.

51. A method of claim 9, wherein the polypeptide has the sequence of SEQ ID NO:23.

52. A method of claim 9, wherein the polypeptide has the sequence of SEQ ID NO:24.

53. A method of claim 9, wherein the polypeptide has the sequence of SEQ ID NO:25.

54. A method of claim 9, wherein the polypeptide has the sequence of SEQ ID NO:26.

55. A diagnostic test for a condition or disease associated with the expression of human enzyme molecules (HEM) in a biological sample comprising the steps of:
a) combining the biological sample with an antibody of claim 10, under conditions suitable for the antibody to bind the polypeptide and form an antibody:polypeptide complex; and
b) detecting the complex, wherein the presence of the complex correlates with the presence of the polypeptide in the biological sample.

56. The antibody of claim 10, wherein the antibody is:
a) a chimeric antibody,
b) a single chain antibody,
c) a Fab fragment,
d) a F(ab′)₂ fragment, or
e) a humanized antibody.

57. A composition comprising an antibody of claim 10 and an acceptable excipient.

58. A method of diagnosing a condition or disease associated with the expression of human enzyme molecules (HEM) in a subject, comprising administering to said subject an effective amount of the composition of claim 57.

59. A composition of claim 57, wherein the antibody is labeled.

60. A method of [symbol] nosing a condition or disease associate [symbol] the expression of human enzyme molecules (HEM) in a subject, comprising administering to said subject an effective amount of the composition of claim 59.

61. A method of preparing a polyclonal antibody with the specificity of the antibody of claim 10 comprising:
a) immunizing an animal with a polypeptide having an amino acid sequence selected from the group consisting of SEQ ID NO:1-26, or an immunogenic fragment thereof, under conditions to elicit an antibody response;
b) isolating antibodies from said animal; and
c) screening the isolated antibodies with the polypeptide, thereby identifying a polyclonal antibody which binds specifically to a polypeptide having an amino acid sequence selected from the group consisting of SEQ ID NO:1-26.

62. An antibody produced by a method of claim 61.

63. A composition comprising the antibody of claim 62 and a suitable carrier.

64. A method of making a monoclonal antibody with the specificity of the antibody of claim 10 comprising:
a) immunizing an animal with a polypeptide having an amino acid sequence selected from the group consisting of SEQ ID NO:1-26, or an immunogenic fragment thereof, under conditions to elicit an antibody response;
b) isolating antibody producing cells from the animal;
c) fusing the antibody producing cells with immortalized cells to form monoclonal antibody-producing hybridoma cells;
d) culturing the hybridoma cells; and
e) isolating from the culture monoclonal antibody which binds specifically to a polypeptide having an amino acid sequence selected from the group consisting of SEQ ID NO:1-26.

65. A monoclonal antibody produced by a method of claim 64.

66. A composition comprising the antibody of claim 65 and a suitable carrier.

67. The antibody of claim 10, wherein the antibody is produced by screening a Fab expression library.

68. The antibody [symbol] aim 10, wherein the antibody is produce [symbol] y screening a recombinant immunoglobulin library.

69. A method for detecting a polypeptide having an amino acid sequence selected from the group consisting of SEQ ID NO:1-26 in a sample, comprising the steps of:
a) incubating the antibody of claim 10 with a sample under conditions to allow specific binding of the antibody and the polypeptide; and
b) detecting specific binding, wherein specific binding indicates the presence of a polypeptide having an amino acid sequence selected from the group consisting of SEQ ID NO:1-26 in the sample.

70. A method of purifying a polypeptide having an amino acid sequence selected from the group consisting of SEQ ID NO:1-26 from a sample, the method comprising:
a) incubating the antibody of claim 10 with a sample under conditions to allow specific binding of the antibody and the polypeptide; and
b) separating the antibody from the sample and obtaining the purified polypeptide having an amino acid sequence selected from the group consisting of SEQ ID NO:1-26.

71. A microarray wherein at least one element of the microarray is a polynucleotide of claim 12.

72. A method for generating a transcript image of a sample which contains polynucleotides, the method comprising the steps of:
a) labeling the polynucleotides of the sample,
b) contacting the elements of the microarray of claim 71 with the labeled polynucleotides of the sample under conditions suitable for the formation of a hybridization complex, and
c) quantifying the expression of the polynucleotides in the sample.

73. An array comprising different nucleotide molecules affixed in distinct physical locations on a solid substrate, wherein at least one of said nucleotide molecules comprises a first oligonucleotide or polynucleotide sequence specifically hybridizable with at least 30 contiguous nucleotides of a target polynucleotide, said target polynucleotide having a sequence of claim 11.

74. An array of claim 73, wherein said first oligonucleotide or polynucleotide sequence is completely complementary to at least 30 contiguous nucleotides of said target polynucleotide.

75. An array of claim 73, wherein said first oligonucleotide of polynucleotide sequence is completely complementary to at least 60 contiguous nucleotides of said target polynucleotide.

76. An array of claim 73, which is a microarray.

77. An array of claim 73, further comprising said target polynucleotide hybridized to said first oligonucleotide or polynucleotide.

78. An array of claim 73, wherein a linker joins at least one of said nucleotide molecules to said solid substrate.

79. An array of claim 73, wherein each distinct physical location on the substrate contains multiple nucleotide molecules having the same sequence, and each distinct physical location on the substrate contains nucleotide molecules having a sequence which differs from the sequence of nucleotide molecules at another physical location on the substrate.

80. A polypeptide of claim 1, comprising the amino acid sequence of SEQ ID NO:1.

81. A polypeptide of claim 1, comprising the amino acid sequence of SEQ ID NO:2.

82. A polypeptide of claim 1, comprising the amino acid sequence of SEQ ID NO:3.

83. A polypeptide of claim 1, comprising the amino acid sequence of SEQ ID NO:4.

84. A polypeptide of claim 1, comprising the amino acid sequence of SEQ ID NO:5.

85. A polypeptide of claim 1, comprising the amino acid sequence of SEQ ID NO:6.

86. A polypeptide of claim 1, comprising the amino acid sequence of SEQ ID NO:7.

87. A polypeptide of claim 1, comprising the amino acid sequence of SEQ ID NO:8.

88. A polypeptide of claim 1, comprising the amino acid sequence of SEQ ID NO:9.

89. A polypeptide of claim 1, comprising the amino acid sequence of SEQ ID NO:10.

90. A polypeptide of claim 1, comprising the amino acid sequence of SEQ ID NO:11.

91. A polypeptide of claim 1, comprising the amino acid sequence of SEQ ID NO:12.

92. A polypeptide of claim 1, comprising the amino acid sequence of SEQ ID NO:13.

93. A polypeptide of claim 1, comprising the amino acid sequence of SEQ ID NO:14.

94. A polypeptide of claim 1, comprising the amino acid sequence of SEQ ID NO:15.

95. A polypeptide of claim 1, comprising the amino acid sequence of SEQ ID NO:16.

96. A polypeptide of claim 1, comprising the amino acid sequence of SEQ ID NO:17.

97. A polypeptide of claim 1, comprising the amino acid sequence of SEQ ID NO:18.

98. A polypeptide of claim 1, comprising the amino acid sequence of SEQ ID NO:19.

99. A polypeptide of claim 1, comprising the amino acid sequence of SEQ ID NO:20.

100. A polypeptide of claim 1, comprising the amino acid sequence of SEQ ID NO:21.

101. A polypeptide of claim 1, comprising the amino acid sequence of SEQ ID NO:22.

102. A polypeptide of claim 1, comprising the amino acid sequence of SEQ ID NO:23.

103. A polypeptide of claim 1, comprising the amino acid sequence of SEQ ID NO:24.

104. A polypeptide of claim 1, comprising the amino acid sequence of SEQ ID NO:25.

105. A polypeptide of claim 1, comprising the amino acid sequence of SEQ ID NO:26.

106. A polynucleotide of claim 11, comprising the polynucleotide sequence of SEQ ID NO:27.

107. A polynucleotide of claim 11, comprising the polynucleotide sequence of SEQ ID NO:28.

108. A polynucleotide of claim 11, comprising the polynucleotide sequence of SEQ ID NO:29.

109. A polynucleotide of claim 11, comprising the polynucleotide sequence of SEQ ID NO:30.

110. A polynucleotide of claim 11, comprising the polynucleotide sequence of SEQ ID NO:31.

111. A polynucleotide of claim 11, comprising the polynucleotide sequence of SEQ ID NO:32.

112. A polynucleotide of claim 11, comprising the polynucleotide sequence of SEQ ID NO:33.

113. A polynucleotide of claim 11, comprising the polynucleotide sequence of SEQ ID NO:34.

114. A polynucleotide of claim 11, comprising the polynucleotide sequence of SEQ ID NO:35.

115. A polynucleotide of claim 11, comprising the polynucleotide sequence of SEQ ID NO:36.

116. A polynucleotide of claim 11, comprising the polynucleotide sequence of SEQ ID NO:37.

117. A polynucleotide of claim 11, comprising the polynucleotide sequence of SEQ ID NO:38.

118. A polynucleotide of claim 11, comprising the polynucleotide sequence of SEQ ID NO:39.

119. A polynucleotide of claim 1 1, comprising the polynucleotide sequence of SEQ ID NO:40.

120. A polynucleotide of claim 11, comprising the polynucleotide sequence of SEQ ID NO:41.

121. A polynucleotide of claim 11, comprising the polynucleotide sequence of SEQ ID NO:42.

122. A polynucleotide of claim 11, comprising the polynucleotide sequence of SEQ ID NO:43.

123. A polynucleotide of claim 11, comprising the polynucleotide sequence of SEQ ID NO:44.

124. A polynucleotide of claim 11, comprising the polynucleotide sequence of SEQ ID NO:45.

125. A polynucleotide of claim 11, comprising the polynucleotide sequence of SEQ ID NO:46.

126. A polynucleotide of claim 11, comprising the polynucleotide sequence of SEQ ID NO:47.

127. A polynucleotide of claim 11, comprising the polynucleotide sequence of SEQ ID NO:48.

128. A polynucleotide of claim 11, comprising the polynucleotide sequence of SEQ ID NO:49.

129. A polynucleotide of claim 11, comprising the polynucleotide sequence of SEQ ID NO:50.

130. A polynucleotide of claim 11, comprising the polynucleotide sequence of SEQ ID NO:51.

131. A polynucleotide of claim 11, comprising the polynucleotide sequence of SEQ ID NO:52.


SEQUENCE LISTING (52 sequences)

SEQ ID NO: 1 - protein, 404 aa, Homo sapiens
  misc_feature: Incyte ID No 2783442CD1
MAESVERLQQRVQELERELAQERSLQVPRSGDGGGGRVRIEKMSSEVVDSNPYSRLMALKRMGIVSDYEKIRTFAVAIVGVGGVGSVTAEMLTRCGIGKLLLFDYDKVELANMNRLFFQPHQAGLSKVQAAEHTLRNINPDVLFEVHNYNITTVENFQHFMDRISNGGLEEGKPVDLVLSCVDNFEARMTINTACNELGQTWMESGVSENAVSGHIQLIIPGESACFACAPPLVVAANIDEKTLKREGVCAASLPTTMGVVAGILVQNVLKFLLNFGTVSFYLGYNAMQDFFPTMSMKPNPQCDDRNCRKQQEEYKKKVAALPKQEVIQEEEEIIHEDNEWGIELVSEVSEEELKNFSGPVPDLPEGITVAYTIPKKQEDSVTELTVEDSGESLEDLMAKMKNM

SEQ ID NO: 2 - protein, 242 aa, Homo sapiens
  misc_feature: Incyte ID No 2116390CD1
MSGCDAREGDCCSRRCGAQDKEHPRYLIPELCKQFYHLGWVTGTGGGISLKHGDEIYIAPSGVQKERIQPEDMFVCDINEKDISGPSPSKKLKKSQCTPLFMNAYTMRGAGAVIHTHSKAAVMATLLFPGREFKITHQEMIKGIKKCTSGGYYRYDDMLVVPIIENTPEEKDLKDRMAHAMNEYPDSCAVLVRRHGVYVWGETWEKAKTMCECYDYLFDIAVSMKKVGLDPSQLPVGENGIV

SEQ ID NO: 3 - protein, 404 aa, Homo sapiens
  misc_feature: Incyte ID No 5785224CD1
MNNIKPLEGVKILDLTRVLAGPFATMNLGDLGAEVIKVERPGAGDDTRTWGPPFVGTESTYYLSVNRNKKSIAVNIKDPKGVKIIKELAAVCDVFVENYVPGKLSAMGLGYEDIDEIAPHIIYCSITGYGQTGPISQRAGYDAVASAVSGLMHITGPENGDPVRPGVAMTDLATGLYAYGAIMAGLIQKYKTGKGLFIDCNLLSSQVACLSHIAANYLIGQKEAKRWGTAHGSIVPYQAFKTKDGYIVVGAGNNQQFATVCKILDLPELIDNSKYKTNHLRVHNRKELIKILSERFEEELTSKWLYLFEGSGVPYGPINNMKNVFAEPQVLHNGLVMEMEHPTVGKISVPGPAVRYSKFKMSEARPPPLLGQHTTHILKEVLRYDDRAIGELLSAGVVDQHETH

SEQ ID NO: 4 - protein, 126 aa, Homo sapiens
  misc_feature: Incyte ID No 1870996CD1
MGHMLLPFRLGLGGPIGSGHQFFPWIHIGDLAGILTHALEANHVHGVLNGVAPSSATNAEFAQTFGAALGRRAFIPLPSAVVQAVFGRQRAIMLLEGQKVIPRRTLATGYQYSFPELGAALKEIVA

SEQ ID NO: 5 - protein, 384 aa, Homo sapiens
  misc_feature: Incyte ID No 138841CD1
MAGAEWKSLEECLEKHLPLPDLQEVKRVLYGKELRKLDLPREAFEAASREDFELQGYAFEAAEEQLRRPRIVHVGLVQNRIPLPANAPVAEQVSALHRRIKAIVEVAAMCGVNIICFQEAWTMPFAFCTREKPPWTEFAESAEDGPTTRFCQKLAKNHDMVVVSPILERDSEHGDVLWNTAVVISNSGAVLGKTRKNHIPRVGDFNESTYYMEGNLGHPVFQTQFGRIAVNICYGRHHPLNWLMYSINGAEIIFNPSATIGALSESLWPIEARNAAIANHCFTCAINRVGTEHFPNEFTSGDGKKAHQDFGYFYGSSYVAAPDSSRTPGLSRSRDGLLVAKLDLNLCQQVNDVWNFKMTGRYEMYARELAEAVKSNYSPTIVKE

SEQ ID NO: 6 - protein, 164 aa, Homo sapiens
  misc_feature: Incyte ID No 1485405CD1
MNPIVVVHGGGAGPISKDRKERVHQGMVRAATVGYGILREGGSAVDAVEGAVVALEDDPEFNAGCGSVLNTNGEVEMDASIMDGKDLSAGAVSAVQCIANPIKLARLVMEKTPHCFLTDQGAAQFAAAMGVPEIPGEKLVTERNKKRLEKEKHEKGAQKTDCQK

SEQ ID NO: 7 - protein, 602 aa, Homo sapiens
  misc_feature: Incyte ID No 2024617CD1
MRSMKALQKALSRAGSHCGRGGWGHPSRSPLLGGGVRHHLSEAAAQGRETPRSHQPQHQDHDSSESGMLSRLGDLLFYTIAEGQERIPIHKFTTALKATGLQTSDPRLRDCMSEMHRVVQESSSGGLLDRDLFRKCVSSNIVLLTQAFRKKFVIPDFEEFTGHVDRIFEDVKELTGGKVAAYIPQLAKSNPDLWGVSLCTVDGQRHSVGHTKIPFCLQSCVKPLTYAISISTLGTDYVHKFVGKEPSGLRYNKLSLNEEGIPHNPMVNAGAIVVSSLIKMDCNKAEKFDFVLQYLNKMAGNEYMGFSNATFQSEKETGDRNYAIGYYLKEKKCFPKGVDMMAALDLYFQLCSVEVTCESGSVMAATLANGGICPITGESVLSAEAVRNTLSLMHSCGMYDFSGQFAFHVGLPAKSAVSGAILLVVPNVMGMMCLSPPLDKLGNSHRGTSFCQKLVSLFNFHNYDNLRHCARKLDPRREGAEIRNKTVVNLLFAAYSGDVSALRRFALSAMDMEQKDYDSRTALHVAAAEGHIEVVKFLIEACKVNPFAKDRWGNIPLDDAVQFNHLEVVKLLQDYQDSYTLSETQAEAAAEALSKENLESMV

SEQ ID NO: 8 - protein, 434 aa, Homo sapiens
  misc_feature: Incyte ID No 4721827CD1
MNGSKNYASRPGTRQPVNNRGRSGNALQDTSGKLRIHKCKGPSDLLTVRQSTRNLYARGFHDKDKECSCRESGYRASRSQRKSQRQFLRNQGTPKYKPRFVHTRQTRSLSVEFEGEIYDINLEEEEELQVLQPRNIAKRHDEGHKGPRDLQASSGGNRGRMLADSSNAVGPPTTVRVTHKCFILPNDSIHCERELYQSARAWKDHKAYIDKEIEALQDKIKNLREVRGHLKRRKPEECSCSKQSYYNKEKGVKKQEKLKSHLHPFKEAAQEVDSKLQLFKENNRRRKKERKEKRRQRKGEECSLPGLTCFTHDNNHWQTAPFWNLGSFCACTSSNNNTYWCLRTVNETHNFLFCEFATGFLEYFDMNTDPYQLTNTVHTVERGILNQLHVQLMELRSCQGYKQCNPRPKNLDVGNKDGGSYDLHRGQLWDGWEG

SEQ ID NO: 9 - protein, 369 aa, Homo sapiens
  misc_feature: Incyte ID No 5406614CD1
MGPGARRQGRIVQGRPEMCFCPPPTPLPPLRILTLGTHTPTPCSSPGSAAGTYPTMGSQALPPGPMQTLIFFDMEATGLPFSQPKVTELCLLAVHRCALESPPTSQGPPPTVPPPPRVVDKLSLCVAPGKACSPAASEITGLSTAVLAAHGRQCFDDNLANLLLAFLRRQPQPWCLVAHNGDRYDFPLLQAELAMLGLTSALDGAFCVDSITALKALERASSPSEHGPRKSYSLGSIYTRLYGQSPPDSHTAEGDVLALLSICQWRPQALLRWVDAHARPFGTIRPMYGVTASARTKPRPSAVTTTAHLATTRNTSPSLGESRGTKDLPPVKDPGALSREGLLAPLGLLAILTLAVATLYGLSLATPGE

SEQ ID NO: 10 - protein, 453 aa, Homo sapiens
  misc_feature: Incyte ID No 1252792CD1
MGRTREAGCVAAGVVIGAGACYCVYRLAWGRDENEKIWDEDEESTDTSEIGVETVKGAKTNAGAGSGAKLQGDSEVKPEVSLGLEDCPGVKEKAHSGSHSGGGLEAKAKALFNTLKEQASAKAGKGARVGTISGNRTLAPSLPCPGGRGGGCHPTRSGSRAGGRASGKSKGKARSKSTRAPATTWPVRRGKFNFPYKIDDILSAPDLQKVLNILERTNDPFIQEVALVTLGNNAAYSFNQNAIRELGGVPIIAKLIKTKDPIIREKTYNALNNLSVNAENQGKIKTYISQVCDDTMVCRLDSAVQMAGLRLLTNMTVTNHYQHLLSYSFPDFFALLFLGNHFTKIQIMKLIINFTENPAMTRELVSCKVPSELISLFNKEWDREILLNILTLFENINDNIKNEGLASSRKEFSRSSLFFLFKESGVCVKKIKALANHNDLVVKVKVLKVLTKL

SEQ ID NO: 11 - protein, 511 aa, Homo sapiens
  misc_feature: Incyte ID No 1299305CD1
MYRLMSAVTARAAAPGGLASSCGRRGVHQRAGLPPPDPEASPLAEPPQEQSLAPWSPQTPAPPCSRCFARAIESSRDLLHRIEDEVGAPGIVVGVSVDGKEVWSEGLGYADVENRVPCKPETVMRIASISKSLTMVALAKLWEAGKLDLDIPVQHYVPEFPEKEYEGEKVSVTTRLLISHLSGIRHYEKDIKKVKEEKAYKALKMMKENVAFEQEKEGKSNEKNDFTKFKTEQENEAKCRNSKPGKKKNDFEQGELYLREKFENSIESLRLFKNDPLFFKPGSQFLYSTFGYTLLAAIVERASGCKYLDYMQKIFHDLDMLTTVQEENEPVIYNRARFYVYNKKKRLVNTPYVDNSYKWAGGGFLSTVGDLLKFGNAMLYGYQVGLFKNSNENLLPGYLKPETMVMMWTPVPNTEMSWDKEGKYAMAWGVVERKQTYGSCRKQRHYASHTGGAVGASSVLLVLPEELDTETINNKVPPRGIIVSIICNMQSVGLNSTALKIALEFDKDRSD

SEQ ID NO: 12 - protein, 520 aa, Homo sapiens
  misc_feature: Incyte ID No 1385190CD1
MSESGHSQPGLYGIERRRRWKEPGSGGPQNLSGPGGRERDYIAPWERERRDASEETSTSVMQKTPIILSKPPAERSKQPPPPTAPAAPPAPAPLEKPIVLMKPREEGKGPVAVTGASTPEGTDPPPPAAPAPPKGEKEGQRPTQPVYQIQNRGMGTAAPAAMDPVVGQAKLLPPERMKHSIKLVDDQMNWCDSAIEYLLDQTDVLVVGVLGLQGTGKSMVMSLLSANTPEEDQRTYVFRAQSAEMKERGGNQTSGIDFFITQERIVFLDTQPILSPSILDHLINNDRKLPPEYNLPHTYVEMQSLQIAAFLFTVCHVVIVVQDWFTDLSLYRFLQTAEMVKPSTPSPSHESSSSSGSDEGTEYYPHLVFLQNKARREDFCPRKLRQMHLMIDQLMAHSHLRYKGTLSMLQCNVFPGLPPDFLDSEVNLFLVPFMDSEAESENPPRAGPGSSPLFSLLPGYRGHPSFQSLVSKLRSQVMSMARPQLSHTILTEKNWFHYAARIWDGVRKSSALAEYSRLLA

SEQ ID NO: 13 - protein, 687 aa, Homo sapiens [sequence omitted]

SEQ ID NO: 14 - protein, 190 aa, Homo sapiens
  misc_feature: Incyte ID No 254567CD1
MARHVFLTGPPGVGKTTLIHKASEVLKSSGVPVDGFYTEEVRQGGRRIGFDVVTLSGTRGPLSRVGLEPPPGKRECRVGQYVVDLTSFEQLALPVLRNADCSSGPGQRVCVIDEIGKMELFSQLFIQAVRQTLSTPGTIILGTIPVPKGKPLALVEEIRNRKDVKVFNVTKENRNHLLPDIVTCVQSSRK

SEQ ID NO: 15 - protein, 284 aa, Homo sapiens
  misc_feature: Incyte ID No 3903488CD1
MPRYAQLVMGPAGSGKSTYCATMVQHCEALNRSVQVVNLDPAAEHFNYSVMADIRELIEVDDVMEDDSLRFGPNGGLVFCMEYFANNFDWLENCLGHVEDDYILFDCPGQIELYTHLPVMKQLVQQLEQWEFRVCGVFLVDSQFMVESFKFISGILAALSAMISLEIPQVNIMTKMDLLSKKAKKEIEKFLDPDMYSLLEDSTSDLRSKKFKKLTKAICGLIDDYSMVRFLPYDQSDEESMNIVLQHIDFAIQYGEDLEFKEPKEREDESSSMFDEYFQECQDE

SEQ ID NO: 16 - protein, 245 aa, Homo sapiens
  misc_feature: Incyte ID No 5391816CD1
MANEAYPCPCDIGHRLEYGGLGREVQVEHIKAYVTKSPVDAGKAVIVIQDIFGWQLPNTRYIADMISGNGYTTIVPDFFVGQEPWDPSGDWSIFPEWLKTRNAQKIDREISAILKYLKQQCHAQKIGIVGFCWGGTAVHHLMMKYSEFRAGVSVYGIVKDSEDIYNLKNPTLFIFAENDVVIPLKDVSLLTQKLKEHCKVEYQIKTFSGQTHGFVHRKREDCSPADKPYIDEARRNLIEWLNKYM

SEQ ID NO: 17 - protein, 231 aa, Homo sapiens
  misc_feature: Incyte ID No 5886989CD1
MFLVGLTGGIASGKSSVIQVFQQLGCAVIDVDVMARHVVQPGYPAHRRIVEVFGTEVLLENGDINRKVLGDLIFNQPDRRQLLNAITHPEIRKEMMKETFKYFLRGYRYVILDIPLLFETKKLLKYMKHTVVVYCDRDTQLARLMRRNSLNRKDAEARINAQLPLTDKARMARHVLDNSGEWSVTKRQVILLHTELERSLEYLPLRFGVLTGLAAIASLLYLLTHYLLPYA

SEQ ID NO: 18 - protein, 475 aa, Homo sapiens [sequence omitted]

SEQ ID NO: 19 - protein, 421 aa, Homo sapiens
  misc_feature: Incyte ID No 1332963CD1
MQALRHVVCALSGGVDSAVAALLLRRRGYQVTGVFMKNWDSLDEHGVCTADKDCEDAYRVCQILDIPFHQVSYVKEYWNDVFSDFLNEYEKGRTPNPDIVCNKHIKFSCFFHYAVDNLGADAIATGHYARTSLEDEEVFEQKHVKKPEGLFRNRFEVRNAVKLLQAADSFKDQTFFLSQVSQDALRRTIFPLGGLTKEFVKKIAAENRLHHVLQKKESMGMCFIGKRNFEHFLLQYLQPRPGHFISIEDNKVLGTHKGWFLYTLGQRANIGGLREPWYVVEKDSVKGDVFVAPRTDHPALYRDLLRTSRVHWIAEEPPAALVRDKMMECHFRFRHQMALVPCVLTLNQDGTVWVTAVQAVRALATGQFAVFYKGDECLGSGKILRLGPSAYTLQKGQRRAGMATESPSDSPEDGPGLSPLL

SEQ ID NO: 20 - protein, 310 aa, Homo sapiens
  misc_feature: Incyte ID No 1559410CD1
MAGAAPTTAFGQAVIGPPGSGKTTYCLGMSEFLRALGRRVAVVNLDPANEGLPYECAVDVGELVGLGDVMDALRLGPNGGLLYCMEYLEANLDWLRAKLDPLRGHYFLFDCPGQVELCTHHGALRSIFSQMAQWDLRLTAVHLVDSHYCTDPAKFISVLCTSLATMLHVELPHINLLSKMDLIEHYGKLAFNLDYYTEVLDLSYLLDHLASDPFFRHYRQLNEKLVQLIEDYSLVSFIPLNIQDKESIQRVLQAVDKANGYCFGAQEQRSLEAMMSAAMGADFHFSSTLGIQEKYLAPSNQSVEQEAMQL

SEQ ID NO: 21 - protein, 194 aa, Homo sapiens
  misc_feature: Incyte ID No 1752587CD1
MEAMWLLCVALAVLAWGFLWVWDSSERMKSREQGGRLGAESRTLLVIAHPDDEAMFFAPTVLGLARLRHWVYLLCFSAGNYYNQGETRKKELLQSCDVLGIPLSSVMIIDNRDFPDDPGMQWDTEHVARVLLQHIEVNGINLVVTFDAGGVSGHSNHIALYAAVRALHSEGKLPKGKACSFCKGPQDTVPLRNL

SEQ ID NO: 22 - protein, 349 aa, Homo sapiens
  misc_feature: Incyte ID No 1911509CD1
MAAEEEEVDSADTGERSGWLTGWLPTWCPTSISHLKEAEEKMLKCVPCTYKKEPVRISNGNKIWTLKFSHNISNKTPLVLLHGFGGGLGLWALNFGDLCTNRPVYAFDLLGFGRSSRPRFDSDAEEVENQFVESIEEWRCALGLDKMILLGHNLGGFLAAAYSLKYPSRVNHLILVEPWGFPERPDLADQDRPIPVWIRALGAALTPFNPLAGLRIAGPFGLSLVQRLRPDFKRKYSSMFEDDTVTEYIYHCNVQTPSGETAFKNMTIPYGWAKRPMLQRIGKMHPDIPVSVIFGARSCIDGNSGTSIQSLRPHSYVKTIAILGAGHYVYADQPEEFNQKVKEICDTVD

SEQ ID NO: 23 - protein, 245 aa, Homo sapiens
  misc_feature: Incyte ID No 2210170CD1
MGIWQRLLLFGGVSLRAGGGATAPLGGSRAMVCGRQLSGAGSETLKQRRTQIMSRGLPKQKPIEGVKQVIVVASGKGGVGKSTTAVNLALALAANDSSKAIGLLDVDVYGPSVPKMMNLKGNPELSQSNLMRPLLNYGIACMSMGFLVEESEPVVWRGLMVMSAIEKLLRQVDWGQLDYLVVDMPPGTGDVQLSVSQNIPITGAVIVSTPQDIALMDAHKGAEMFRRVHVPVSVYSFTVKNIKLF

SEQ ID NO: 24 - protein, 255 aa, Homo sapiens [sequence omitted]

SEQ ID NO: 25 - protein, 377 aa, Homo sapiens
  misc_feature: Incyte ID No 2884114CD1
MEEPEEPADSGQSLVPVYIYSPEYVSMCDSLAKIPKRASMVHSLIEAYALHKQMRIVKPKVASMEEMATFHTDAYLQHLQKVSQEGDDDHPDSIEYGLGYDCPATEGIFDYAAAIGGATITAAQCLIDGMCKVAINWSGGWHHAKKDEASGFCYLNDAVLGILRLRRKFERILYVDLDLHHGDGVEDAFSFTSKVMTVSLHKFSPGFFPGTGDVSDVGLGKGRYYSVNVPIQDGIQDEKYYQICESVLKEVYQAFNPKAVVLQLGADTIAGDPMCSFNMTPVGIGKCLKYILQWQLATLILGGGGYNLANTARCWTYLTGVILGKTLSSEIPDHEFFTAYGPDYVLEITPSCRPDRNEPHRIQQILNYIKGNLKHVV

SEQ ID NO: 26 - protein, 403 aa, Homo sapiens
  misc_feature: Incyte ID No 4103559CD1
MELSYQTLKFTHQAREACEMRTEARRKNLLILISHYLTQEGYIDTANALEQETKLGLRRFEVCDNIDLETILMEYESYYFVKFQKYPKIVKKSSDTENNLPQRCRGKTRRMMNDSCQNLPKINQQRPRSKTTAGKTGDTKSLNKEHPNQEVVDNTRLESANFGLHISRIRKDSGEENAHPRRGQIIDFQGLLTDAIKGATSELALNTFDHNPDPSERLLKPLSAFIGMNSEMRELAAVVSRDIYLHNPNIKWNDIIGLDAAKQLVKEAVVYPIRYPQLFTGILSPWKGLLLYGPPGTGKTLLAKAVATECKTTFFNISASTIVSKWRGDSEKLVRVLFELARYHAPSTIFLDELESVMSQRGTASGGEHEGSLRMKTELLVQMDGLARSEDLVFVLAASNLPW

SEQ ID NO: 27 - DNA, 1478 bp, Homo sapiens
  misc_feature: Incyte ID No 2783442CB1
ctccgaggaaggcctgtgggagtctcggagacgtgtctgtctgtgaggcgctgggtgcacgtccccagggctctgggctaggaaggcagcggcgaggtgcctccccacgtacccctcgcgggcccagccgagcaacgtggggcgaaggcggcggcgaaggcccgggctgggagcgttggcggccggagtcccagccatggcggagtctgtggagcgcctgcagcagcgggtccaggagctggagcgggaacttgcccaggagaggagtctgcaggtcccgaggagcggcgacggagggggcggccgggtccgcatcgagaagatgagctcagaggtggtggattcgaatccctacagccgcttgatggcattgaaacgaatgggaattgtaagcgactatgagaaaatccgtacctttgccgtagcaatagtaggtgttggtggagtaggtagtgtgactgctgaaatgctgacaagatgtggcattggtaagttgctactctttgattatgacaaggtggaactagccaatatgaatagacttttcttccaacctcatcaagcaggattaagtaaagttcaagcagcagaacatactctgaggaacattaatcctgatgttctttttgaagtacacaactataatataaccacagtggaaaactttcaacatttcatggatagaataagtaatggtgggttagaagaaggaaaacctgttgatctagttcttagctgtgtggacaattttgaagctcgaatgacaataaatacagcttgtaatgaacttggacaaacatggatggaatctggggtcagtgaaaatgcagtttcagggcatatacagcttataattcctggagaatctgcttgttttgcgtgtgctccaccacttgtagttgctgcaaatattgatgaaaagactctgaaacgagaaggtgtttgtgcagccagtcttcctaccactatgggtgtggttgctgggatcttagtacaaaacgtgttaaagtttctgttaaattttggtactgttagtttttaccttggatacaatgcaatgcaggatttttttcctactatgtccatgaagccaaatcctcagtgtgatgacagaaattgcaggaagcagcaggaggaatataagaaaaaggtagcagcactgcctaaacaagaggttatacaagaagaggaagagataatccatgaagataatgaatggggtattgagctggtatctgaggtttcagaagaggaactgaaaaatttttcaggtccagttccagacttacctgaaggaattacagtggcatacacaattccaaaaaagcaagaagattctgtcactgagttaacagtggaagattctggtgaaagcttggaagacctcatggccaaaatgaagaatatgtagataatggactgggatatattgtatttctcatgttaaagcctctgcccttgaaattaaaaaagaatta

SEQ ID NO: 28 - DNA, 1183 bp, Homo sapiens
  misc_feature: Incyte ID No 2116390CB1
cccacgcgtccgcccacgcgtccgatcccaggctaagcgccgcgcgcaaagccgtgcggagattggaggccgcgcgggtccctggtctgggccatgtctggctgtgatgctcgggagggagactgttgttcccggagatgcggcgcgcaggacaaggagcatccaagatacctgatcccagaactttgcaaacagttttaccatttaggctgggtcactgggactggaggaggaattagcttgaagcatggcgatgaaatctacattgctccttcaggagtgcaaaaggaacgaattcagcctgaagacatgtttgtttgtgatataaatgaaaaggacataagtggaccttcgccatcgaagaagctaaaaaaaagccagtgtactcctcttttcatgaatgcttacacaatgagaggagcaggtgcagtgattcatacccactctaaagctgctgtgatggccacccttctctttccaggacgggagtttaaaattacacatcaagagatgataaaaggaataaagaaatgtacttccggagggtattatagatatgatgatatgttagtggtacccattattgagaatacacctgaggagaaagacctcaaagatagaatggctcatgcaatgaatgaatacccagactcctgtgcagtactggtcagacgtcatggagtatatgtgtggggggaaacatgggagaaggccaaaaccatgtgtgagtgttatgactatttatttgatattgccgtatcaatgaagaaagtaggacttgatccttcacagctcccagttggagaaaatggaattgtctaagccaaaagaaagtctaattatatacagagataaagctaaacgtaattattatttaaatgaaagctatttttttaaatgaattgaaatttttcatgatgctactaatttgccactaaatactgcaaatggtcaccctgaatctcttctgacattggatgttatttgcttatattcttataattttaaatgagggcacagtgaaatgaaaattttatactctatgtttctgtttatttttaaatccttaacagcaaaatatttgcctttaatttcttttttatatatactctcagagaattcctcttaatttttaaagatgctggtgataataaaattcattagaaaatttaaaaaaaaaaaa

SEQ ID NO: 29 - DNA, 1572 bp, Homo sapiens
  misc_feature: Incyte ID No 5785224CB1
ggcgacgctggcgagggtggcagctctgcgcagaacctgcctcttctccggccggggcggcgggagggggctgtggactggccgcccgcagtcagatatgaacaatataaagccattggaaggggtaaaaattctggatctaacaagagtcctggcgggaccttttgctactatgaatttaggagatcttggagcagaagttataaaagtggagagaccaggagctggtgatgatacacgaacttgggggccaccttttgttgggacagaaagtacatattatctcagtgttaaccgaaataaaaaaagtattgctgttaatatcaaggatccaaaaggggtgaaaatcatcaaagagcttgcagctgtttgtgatgtgtttgtggaaaactatgtccctggcaaactgtctgcaatgggcctgggatatgaagatatagacgagattgctcctcacatcatctattgttccatcacagggtatggtcagacaggtccaatttctcagcgagctggttatgatgctgttgcctcggctgtttctggtctgatgcacatcacagggcctgagaatggagatccagttcgcccaggagtagctatgactgatcttgccactggcctgtatgcatatggagctattatggctggattgatacaaaaatacaaaactgggaaaggactgttcattgattgtaacctactgtcatcccaggtggcgtgtttgtctcacatagctgcaaattatcttattggtcaaaaggaagcaaaacgttggggtacagctcatggcagtatcgttccttaccaggcttttaaaaccaaggatggctatattgtagttggagcaggaaataaccagcagtttgccaccgtctgcaagatcttggatttgcctgagttgattgataattccaagtataaaactaaccaccttcgggtacacaatagaaaagagcttattaaaatattatctgaacggtttgaagaagaactgaccagcaagtggttatatctttttgaaggcagtggagtcccgtatggcccaatcaacaacatgaagaatgtatttgcagaacctcaggtattacacaatggcctcgttatggagatggagcatccaactgtggggaagatttccgtcccaggcccagctgtgagatacagtaagttcaagatgtcagaggccaggccgcccccgctgctcgggcagcacacaacgcacatcctgaaggaggtcctgagatacgatgacagggccatcggggagctgctcagcgctggagtggtggaccaacatgaaactcactgacaaaggaaaagggctcttcctcataacctcgatccgaatacactggcaaaggcaacactttgcttggacccttctccccagttctgataccactaaaaagaagatttagagtaactccagatttcttacatggcatctccagaatggctctggtattaatgaatctagtgccttttaaatgtatcccacgttttgttccctaccatcttttttttcagatgatgatttcattatggatttgtgggatttttaaaaataaa

SEQ ID NO: 30 - DNA, 1335 bp, Homo sapiens
  misc_feature: Incyte ID No 1870996CB1
ggacggaaggcgcccccaccccaactccacgggaatataaacaatttgtattttccgatcaggtggcgggacaggcttcattgggacagccctaacccagctgctgaatgccagaggccacgaagtgacgttggtctcccgaaagcccgggcccggccggatcacgtgggatgagctcgctgcatcggggctgccgagctgcgatgccgccgtcaacctggccggagagaacatcctcaaccctctccgaaggttcagcccgggccctaaagctgatacccactagagcacagggaggacagtgccccactgatgagaacctgtgagctatggatggaatgaaaccttccaaaaagaggttctcggcagccgcctagagaccacccaattgctggctaaagccatcaccaaagccccacaaccccccaaggcctgggtcttagtcacaggtgtagcttactaccagcccagtctgactgcggagtatgatgaagacagcccaggaggggactttgactttttctccaacctcgtaaccaaatgggaagctgcagccaggcttcctggagattctacacgccaggtggtggtgcgctcaggggttgtgctgggccgtgggggtggtgccatgggccacatgctgctgccctttcgcctgggcctggggggccccatcggctcaggccaccaattcttcccctggatacacatcggggacctggcaggaatcctgacccatgcccttgaagcaaaccacgtgcacggggtcctgaatggagtggctccatcctccgccactaatgctgagtttgcccagaccttcggtgctgccctgggccgccgagccttcatccctctccccagcgctgtggtgcaagctgtctttgggcgacagcgtgccatcatgctgctggagggccagaaggtgatcccacggcgaacactggccactggctaccagtattccttcccagagctaggggctgccttaaaggaaattgtagcctaagtaggtcatggcaagggcctgaggcctgttcctcacaggcttccaggttaggcactgtgaataggctcagctcctctagagagctgaagccatctggttcttagattcctctcccagtcctctttcccattgttctgttgctccaccttattgtctcaaggccgtaatctcatcaggttgggacattaatcttttcaactccttgtaagatttcccggtttggtttctctacatgtcctgcagctgccccacttctcctttacgctgtgtagagaatgctctgcagtttaggcaataaaaataaattgtctcactaaaaaaaaaaa

SEQ ID NO: 31 - DNA, 1341 bp, Homo sapiens
  misc_feature: Incyte ID No 138841CB1
gcaaagggcaggcagttcgtgcgcggacacaagcactggcggaccgtggccatggcgggcgctgagtggaagtcgctggaggaatgcttggagaagcacctgccgctccccgacttgcaggaagtgaagcgcgttctctatggcaaggaactcaggaagcttgatctgcccagggaagctttcgaagctgcctccagagaagactttgaactgcagggatatgcctttgaagcagcggaggagcagctgagacgaccccgcattgtgcacgtggggctggttcagaacagaatccccctccccgcaaatgcccctgtggcagaacaggtctctgcccttcatagacgcataaaggctatcgtagaggtggctgcaatgtgtggagtcaacatcatctgtttccaggaagcatggactatgccctttgccttctgtacgagagagaagcctccttggacagaatttgctgagtcagcagaggatgggcccaccaccagattctgtcagaagctggcgaagaaccatgacatggtggtggtgtctcccatcctggaacgagacagcgagcatggggatgttttgtggaatacagccgtggtgatctccaattccggagcagtcctgggaaagaccaggaaaaaccacatccccagagtgggtgatttcaacgagtcaacttactacatggagggaaacctgggccaccccgtgttccagacgcagttcggaaggatcgcggtgaacatttgctacgggcggcaccaccccctcaactggcttatgtacagcatcaacggggctgagatcatcttcaacccctcggccacgataggagcactcagcgagtccctgtggcccatcgaggccagaaacgcagccattgccaatcactgcttcacctgcgccatcaatcgagtgggcaccgagcacttcccgaacgagtttacctcgggagatggaaagaaagctcaccaggactttggctacttttatggctcgagctatgtggcagcccctgacagcagccggactcctgggctgtcccgtagccgggatggactgctagttgctaagctcgacctaaacctctgccagcaggtgaatgatgtctggaacttcaagatgacgggcaggtatgagatgtacgcacgggagctcgccgaagctgtcaagtccaactacagccccaccatcgtgaaagagtagccggcttcagtgcctgccttggggtgaggaagacacctctgccccagtggattagcaagtgtggcaggcttaacatgtccaggttctccccaataacattgtccaggttggttttaaaattcccaggcaagggga

SEQ ID NO: 32 - DNA, 755 bp, Homo sapiens
  misc_feature: Incyte ID No 1485405CB1
ggctgagcggtttcgagccggcgtcggggagcggcggtaccgggcggctgcggggctggctcgacccagcttgaggtctcggcgtccgcgtcctgcggtgccctggggtctcccgaggaccttgtacccgcgcggtttccttgggctggctttggacgacgctttcgccttcctgctgcctaggatccgccgacatgaatcccatcgtagtggtccacggcggcggagccggtcccatctccaaggatcggaaggagcgagtgcaccagggcatggtcagagccgccaccgtgggctacggcatcctccgggagggcgggagcgccgtggatgccgtagagggagctgtcgtcgccctggaagacgatcccgagttcaacgcaggttgtgggtctgtcttgaacacaaatggtgaggttgaaatggatgctagtatcatggatggaaaagacctgtctgcaggagcagtgtccgcagtccagtgtatagcaaatcccattaaacttgctcggcttgtcatggaaaagacacctcattgctttctgactgaccaaggcgcagcgcagtttgcagcagctatgggggttccagagattcctggagaaaaactggtgacagagagaaacaaaaagcgcctggaaaaagagaagcatgaaaaaggtgctcagaaaacagattgtcaaaagtaagtcttacctgtggctcgcattatttgggagttattaaaatatgaaagtttggcaaataaaaaaaaa

SEQ ID NO: 33 - DNA, 2390 bp, Homo sapiens
  misc_feature: Incyte ID No 2024617CB1
ggcgcacctgcagagccggaggttgctggggcatgcgctccatgaaggctctgcagaaggccctgagccgggctggcagtcactgcgggcgaggaggctggggtcacccgagccggagccccctccttggcgggggcgtccggcaccacctcagtgaggccgcggcgcagggcagagagacgccacgcagccaccagccgcagcaccaggatcatgattcatcagaaagtggcatgctgtcccgcctgggtgatttgctcttttacactattgctgaaggacaggaacgaatccctatccacaagttcaccactgcactaaaggccactggactgcagacatcagatcctcggctccgagactgcatgagcgagatgcaccgcgtggtccaagaatccagtagtggtggcctcttggaccgagatctcttccgaaagtgtgtgagcagcaacattgtgctcctgacccaggcattccgaaagaagtttgtcattcctgattttgaggagttcacgggccatgtggatcgcatctttgaggatgtcaaagagctcactggaggcaaagtggcagcctacatccctcagctggccaagtcaaacccagacctgtggggtgtctccctgtgcactgtggatggtcaacggcactctgtgggccacacaaagatccccttctgcctgcagtcctgtgtgaagcccctcacctatgccatctccataagcaccctaggcactgactacgtgcacaagtttgtgggcaaagagccaagtggcctgcgctacaacaagctctccctcaatgaggaaggaatcccccataaccccatggtcaatgctggtgccattgttgtcagctccctgatcaagatggactgtaacaaagcagagaagtttgattttgtgttgcagtatctcaacaaaatggctgggaatgaatacatgggtttcagcaatgccacattccagtcagagaaggaaacaggggatcggaattatgccatcggctattatctcaaggaaaagaagtgctttcctaagggggtggacatgatggctgcccttgatctctacttccagctgtgttctgtggaggtcacttgtgaatcaggcagtgtcatggcagccaccctcgccaacggtgggatctgccccatcacaggcgagagtgtgctgagtgctgaagcagtgcgcaacaccctcagcctcatgcattcctgcggcatgtatgacttctctggccagtttgccttccacgtgggcctgccagccaagtcagctgtatcaggagccatcctcctggtggtacccaatgtcatgggaatgatgtgcctgtcacccccattggacaagctggggaacagccatagggggaccagcttctgccagaagttggtgtctctcttcaatttccacaactatgacaacctgaggcactgtgctcggaagttagacccacggcgtgaaggggcagaaattcggaacaagactgtggtcaacctgttatttgctgcctatagtggcgatgtctcagctcttcgaaggtttgccttgtcagccatggatatggaacagaaagactatgactcgcgcacagctctgcatgttgctgcagctgaaggacacatcgaagttgttaaattcctgatcgaggcttgcaaagtgaatccttttgccaaggacaggtggggcaacattcccctggatgatgctgtgcagttcaaccatctggaggtggtcaaactgcttcaagattaccaggactcctacacactctctgaaactcaggctgaggcagcagctgaggccctgtccaaagagaacttagaaagcatggtatgagcacaggtcatggacagcccctgctcaagaaaaagcatgagctggccacacatgtaatccataaccaccaaaaatactatggagagctacactgcttcagtggggaccaagcagtcatttggtgacttaggctagtgctttctatgggagtcaaaataccccattccctcagcagacagagtacagagaagggcctcagaggacacctgcagtacagctatccagagagactgggcttcaaggtacagcctaatggcttgccccactcaaaaccatcccagctcttcacccaggtctcctcttcctctccctgaagaaaccatcatgagagagatactctggtggagggactctagctaccatgcacatgtacatatccacagaatatgggaagtgggaatggctatatacatggctttagtagtctggagaaatctactccccttggccaggacatgctgctgctactgctaacagccaattttatagacagagaaagtattttgtgttcaaataaactttaattaccaaatcaaaaaaaaaaaaaaa

SEQ ID NO: 34 - DNA, 2940 bp, Homo sapiens
  misc_feature: Incyte ID No 4721827CB1
caaaatttggcgtgatacattcctagtggaaagaggcaaatttctacgtaagaaggaagaatccagcaagaatatccaacagtcaaatcacttgcccaaatatgaacgggtcaaagaactatgccagcaggccaggtaccagacagcctgtgaacaaccggggcagaagtggcaatgcattgcaggatacatctggcaagcttcgaattcacaagtgtaaaggacccagtgacctgctcacagtccggcagagcacgcggaacctctacgctcgcggcttccatgacaaagacaaagagtgcagttgtagggagtctggttaccgtgccagcagaagccaaagaaagagtcaacggcaattcttgagaaaccaggggactccaaagtacaagcccagatttgtccatactcggcagacacgttccttgtccgtcgaatttgaaggtgaaatatatgacataaatctggaagaagaagaagaattgcaagtgttgcaaccaagaaacattgctaagcgtcatgatgaaggccacaaggggccaagagatctccaggcttccagtggtggcaacaggggcaggatgctggcagatagcagcaacgccgtgggcccacctaccactgtccgagtgacacacaagtgttttattcttcccaatgactctatccattgtgagagagaactgtaccaatcggccagagcgtggaaggaccataaggcatacattgacaaagagattgaagctctgcaagataaaattaagaatttaagagaagtgagaggacatctgaagagaaggaagcctgaggaatgtagctgcagtaaacaaagctattacaataaagagaaaggtgtaaaaaagcaagagaaattaaagagccatcttcacccattcaaggaggctgctcaggaagtagatagcaaactgcaacttttcaaggagaacaaccgtaggaggaagaaggagaggaaggagaagagacggcagaggaagggggaagagtgcagcctgcctggcctcacttgcttcacgcatgacaacaaccactggcagacagccccgttctggaacctgggatctttctgtgcttgcacgagttctaacaataacacctactggtgtttgcgtacagttaatgagacgcataattttcttttctgtgagtttgctactggctttttggagtattttgatatgaatacagatccttatcagctcacaaatacagtgcacacggtagaacgaggcattttgaatcagctacacgtacaactaatggagctcagaagctgtcaaggatataagcagtgcaacccaagacctaagaatcttgatgttggaaataaagatggaggaagctatgacctacacagaggacagttatgggatggatgggaaggttaatcagccccgtctcactgcagacatcaactggcaaggcctagaggagctacacagtgtgaatgaaaacatctatgagtacagacaaaactacagacttagtctggtggactggactaattacttgaaggatttagatagagtatttgcactgcctgaagagtcactatgagcaaaataaaacaaataagactcaaactgctcaaagtgacgggttcttggttgtctctgctgagcacgctgtgtcaatggagatggcctctgctgactcagatgaagacccaaggcataaggttgggaaaacacctcatttgaccttgccagctgaccttcaaaccctgcatttgaaccgaccaacattaagtccagagagtaaacttgaatggaataacgacattccagaagttaatcatttgaattctgaacactggagaaaaaccgaaaaatggacggggcatgaagagactaatcatctggaaaccgatttcagtggcgatggcatgacagagctagagctcgggcccagccccaggctgcagcccattcgcaggcacccgaaagaacttccccagtatggtggtcctggaaaggacatttttgaagatcaactatatcttcctgtgcattccgatggaatttcagttcatcagatgttcaccatggccaccgcagaacaccgaagtaattccagcatagcggggaagatgttgaccaaggtggagaagaatcacgaaaaggagaagtcacagcacctagaaggcagcgcctcctcttcactctcctctgattagatgaaactgttaccttaccctaaacacagtatttctttttaacttttttatttgtaaactaataaaggtaatcacagccaccaacattccaagctaccctgggtacctttgtgcagtagaagctagtgagcatgtgagcaagcggtgtgcacacggagactcatcgttataatttactatctgccaagagtagaaagaaaggctggggatatttgggttggcttggttttgattttttgcttgtttgtttgttttgtactaaaacagtattatcttttgaatatcgtagggacataagtatatacatgttatccaatcaagatggctagaatggtgcctttctgagtgtctaaaacttgacacccctggtaaatctttcaacacacttccactgcctgcgtaatgaagttttgattcatttttaaccactggaatttttcaatgccgtcattttcagttagatgattttgcactttgagattaaaatgccatgtctatttgattagtcttatttttttatttttacaggcttatcagtctcactgttggctgtcattgtgacaaagtcaaataaacccccaaggacgacacacagtatggatcacatattgtttgacattaagcttttgccagaaaatgttgcatgtgttttacctcgacttgctaaaatcgattagcagaaaggcatggctaataatgttggtggtgaaaataaataaataagtaaacaaaaaaaaaaaaaaaaa

SEQ ID NO: 35 - DNA, 1417 bp, Homo sapiens
  misc_feature: Incyte ID No 5406614CB1
tgctgccagcgagagccgcgggagagtgtgcagccgagtcactactgcctgcctgcctgcctgctacggtgagtgtggcccccacaatgggatggcgcagggcaggagggccatgggttcccccaccccagactaagggggcactaggggaggggccgagtcatgtgaagagggagaccctctcagacagtcgaatgtgctggtcccactaaggaaaccacctcaccctctccaacttcctgcctgaaaatgggccctggagctcgcagacagggcaggattgtgcagggaaggcctgagatgtgcttctgcccaccccctaccccactccctccccttcggatcttaacactgggcactcacacacccaccccatgctcctctccaggctcagcagcaggtacgtacccaaccatgggctcgcaggccctgcccccggggcccatgcagaccctcatctttttcgacatggaggccactggcttgcccttctcccagcccaaggtcacggagctgtgcctgctggctgtccacagatgtgccctggagagcccccccacctctcaggggccacctcccacagttcctccaccaccgcgtgtggtagacaagctctccctgtgtgtggctccggggaaggcctgcagccctgcagccagcgagatcacaggtctgagcacagctgtgctggcagcgcatgggcgtcaatgttttgatgacaacctggccaacctgctcctagccttcctgcggcgccagccacagccctggtgcctggtggcacacaatggtgaccgctacgacttccccctgctccaagcagagctggctatgctgggcctcaccagtgctctggatggtgccttctgtgtggatagcatcactgcgctgaaggccctggagcgagcaagcagcccctcagaacacggcccaaggaagagctacagcctaggcagcatctacactcgcctgtatgggcagtcccctccagactcgcacacggctgagggtgatgtcctggccctgctcagcatctgtcagtggagaccacaggccctgctgcggtgggtggatgctcacgccaggcctttcggcaccatcaggcccatgtatggggtcacagcctctgctaggaccaagccaagaccatctgctgtcacaaccactgcacacctggccacaaccaggaacactagtcccagccttggagagagcaggggtaccaaggatcttcctccagtgaaggaccctggagccctatccagggaggggctgctggccccactgggtctgctggccatcctgaccttggcagtagccacactgtatggactatccctggccacacctggggagtaggccaagaaggaaaatctgacgaataaagacccccgctgccccataaaaaaaaaaaaaa

SEQ ID NO: 36 - DNA, 2133 bp, Homo sapiens
  misc_feature: Incyte ID No 1252792CB1
cgtttggtccggttgcactcttcctatagcccagagggcgagagggcctgtggcctgggggaaggaggacgaggttctgcctggatcccagcagtaggacgctgtgccatttgggaacaaaggaatagtctgcctggaatccctgcagatcttggggccggaggccagtccaacccttggagcaggaagaaacgcaaagttgtcaagaaccaagtcgagctgcctcagagccggcccgcagtagctgcagactccgcccgcgacgtgtgcgcgcttctctgggccagagcgagcctgttttgtgctcgggttaagagatttgtcccagctataccatgggccgcactcgggaagctggctgcgtggccgctggtgtggttatcggggctggtgcctgctactgtgtatacagactggcttggggaagagacgagaacgagaaaatctgggacgaagacgaggagtctacggacacctcagagattggggttgagactgtgaaaggagctaaaactaacgctggggcagggtctggggccaaacttcagggtgattcagaggtcaagcctgaggtgagtttgggactcgaggattgtccgggtgtaaaagagaaggcccattcaggatcccacagcggaggtggcctagaggccaaggccaaggcccttttcaacacgctgaaggaacaggcaagtgcaaaggcaggcaaaggggctagggtgggtaccatctctgggaacaggacccttgcaccgagtttaccctgcccaggaggcaggggtggaggctgccaccccaccaggagtggatctagggccgggggcagggcaagtggaaaatccaagggaaaggcccgaagtaagagcaccagggctccagctacaacatggcctgtccggagaggcaagttcaactttccttataaaattgatgatattctgagtgctcccgacctccaaaaggtcctcaacatcctggagcgaacaaatgatccttttattcaagaagtagccttggtcactctgggtaacaatgcagcatattcatttaaccagaatgccatacgtgaattgggtggtgtcccaattattgcaaaactgataaaaacaaaagaccccataattagggaaaagacttacaatgcccttaataacttgagtgtgaacgcagaaaatcagggcaagattaagacgtacatcagtcaagtgtgtgatgacaccatggtctgtcgcttggactcagctgtgcagatggctgggctaagactgttaaccaacatgactgtgactaatcattaccaacatttgctttcctattcttttccagacttttttgctttgttattcctgggaaatcacttcaccaagatacagattatgaaactaattataaactttactgaaaatccagccatgacaagagagctggtcagttgtaaagtaccatcagaattgatttccctctttaataaagaatgggatagagagattcttcttaatatccttaccctatttgagaatataaatgacaacataaaaaatgaagggctcgcatcatccaggaaagaattcagcagaagttcactttttttcttattcaaagagtctggagtttgtgttaagaaaatcaaagcactagcaaatcacaatgatctggtggtgaaagtaaaagtcctgaaagtattaaccaaactctaatttggagtctgtcccaaacaatattgagatatttgcagttggtacgatgtgatttgtaaattctttgtttttcattgtgcgtatatggtaaagagatcttttcagctgctattttggaataatgactatcatatatcataacagtgactgatgttggttgtaatggttgggtttaggatgaaccattttaaggatgccaaatgaaatattagtatttgtacacagaaagaatttattgatttgatcttattacctagattgagattttttaatctttcctctacctaaactgacaatgaattggttatacatcatgcataagctacacttttatattagtttatatttgttattctaagacttgtgtttcatcaataaagttgtgttttaagcagcagaaaaaaaaaaaaaaagggcggccgctcgcgatctagaa

SEQ ID NO: 37 - DNA, 1829 bp, Homo sapiens
  misc_feature: Incyte ID No 1299305CB1
gcggccggctgtgcagagacgccatgtaccggctcatgtcagcagtgactgcccgggctgccgcccccgggggcttggcctcaagctgcggacgacgcggggtccatcagcgcgccgggctgccgcctcccgaccctgaggcgtcgcctctggccgagccgccacaggagcagtccctcgccccgtggtctccgcagaccccggcgccgccctgctccaggtgcttcgccagagccatcgagagcagccgcgacctgctgcacaggatcgaggatgaggtgggcgcaccgggcatagtggttggagtttctgtagatggaaaagaagtctggtcagaaggtttaggttatgctgatgttgagaaccgtgtaccatgtaaaccagagacagttatgcgaattgctagcatcagcaaaagtctcaccatggttgctcttgccaaattgtgggaagcagggaaactggatcttgatattccagtacaacattatgttcccgaattcccagaaaaagaatatgaaggtgaaaaggtttctgtcacaacaagattactgatttcccatttaagtggaattcgtcattatgaaaaggacataaaaaaggtgaaagaagagaaagcttataaagccttgaagatgatgaaagagaatgttgcatttgagcaagaaaaagaaggcaaaagtaatgaaaagaatgattttactaaatttaaaacagagcaggagaatgaagccaaatgccggaattcaaaacctggcaagaaaaagaatgattttgaacaaggcgaattatatttgagagaaaagtttgaaaattcaattgaatccctaagattatttaaaaatgatcctttgttcttcaaacctggtagtcagtttttgtattcaacttttggctataccctactggcagccatagtagagagagcttcaggatgtaaatatttggactatatgcagaaaatattccatgacttggatatgctgacgactgtgcaggaagaaaacgagccagtgatttacaatagagcaagattttatgtttacaataaaaagaaacgtcttgtcaacacaccttacgtggataactcctataaatgggctggtggtggatttctgtctacagtgggtgaccttctgaaatttgggaatgcaatgctttatggttaccaagttgggctgtttaagaactcaaatgaaaatcttttacctggatacctcaaaccagaaacaatggttatgatgtggaccccagtccctaacacagagatgtcttgggataaagagggtaaatatgcaatggcgtggggtgttgtggaaaggaaacaaacgtatggttcgtgtagaaagcaacggcattatgcttcacatactggaggggcagtgggtgccagtagtgtcctgctggtccttcctgaagaactggatacagagactataaataacaaggttcccccaagaggaatcattgtttctatcatatgtaacatgcaatctgttggcctcaatagcaccgctttgaagattgcccttgaatttgataaagacagatcagactgataaccttaacaccataggtgcaaaatgagttgttctgaggtttttttgaaacattaaagttccaaaacatgacatttttaagaataaatttgaaatagagtataattgaatgcagagaattatgtacctctaattgcttaattttgtaatggtcttttattgtagaattggttctttatactcagggaagtaattatattgtttttactttttgaaaaaagtgttaactcttgaaataaaatattctgataaaataaaaaaaaaaaaaaa

SEQ ID NO: 38 - DNA, 2323 bp, Homo sapiens
  misc_feature: Incyte ID No 1385190CB1
cgagctccagccctcagcgcatgcgcaagacgagtcgcctgagggaactgatctcagctcgggcccgcgttacatcctcctcctcttcttccttcggcccagctttccttaggggctgcaacccggacgccgaggccggtttcggagtggggagtgcccattttctctccttcccacgttcctggcccccagacgccatttgcaggcgggtggcttgggtcagcctccccgcccccacccgactcccgtcacgggagagcgcacaccgcgccccgagaaccaatcagcagccgcgttaggtaaccatgtctgagtctggacacagtcagcctggactctatgggatagagcggcggcgacggtggaaggagcctggctctggtggcccccagaatctctctgggcctggtggtcgggagagggactacattgcaccatgggaaagagagagaagggatgccagcgaagagacaagcacttccgtcatgcagaaaacccccatcatcctctcaaaacctccagcagagcggtcaaaacagccaccacctccaacagcccctgctgccccgcctgctccagcccctctggagaagcccatcgttctcatgaagccacgggaggaggggaaggggcctgtggccgtgacaggtgcctctacccctgagggcaccgacccaccaccccctgcagcccctgcgccacccaagggggagaaggaggggcagagacccacacagcctgtgtaccagatccagaaccggggcatgggcactgccgcaccagcagccatggaccctgtcgtgggtcaggccaaactactgcccccagagcgcatgaagcacagcatcaagttggtggatgaccagatgaattggtgtgacagtgccatcgagtacctgttggatcagactgatgtgttggtggttggtgtcctgggcctccaggggacaggcaagtccatggtcatgtcattgttgtcagccaacactccagaggaggaccagaggacttatgttttccgggcccagagcgctgaaatgaaggaacgagggggcaaccagaccagtggcatcgacttctttattacccaagaacggattgttttcctggacacacagcccatcctgagcccttctatcctagaccatctcatcaataatgaccgcaaactgcctccagagtacaaccttccccacacttacgttgaaatgcagtcactccagattgctgccttccttttcacggtctgccatgtggtgattgttgtccaggactggttcacagacctcagtctctacaggttcctgcagacagcagagatggtgaagccctccaccccatcccccagccacgagtccagcagctcatcgggctccgatgaaggcaccgagtactacccccacctagtcttcttgcagaacaaagctcgccgagaggacttctgtcctcggaagctgcggcagatgcacctgatgattgaccagctcatggcccactcccacctgcgttacaagggaactctgtccatgttacaatgcaatgtcttcccggggcttccacctgacttcctggactctgaggtcaacttattcctggtacccttcatggacagtgaagcagagagtgaaaacccaccaagagcaggacctggttccagcccactcttctccctgctgcctgggtatcgtggccaccccagtttccagtccttggtgagcaagctccggagccaagtgatgtccatggcccggccacagctgtcacacacgatcctcaccgagaagaactggttccactacgctgcccggatctgggatggggtgagaaagtcctctgctctggcagagtacagccgcctgctggcctgaggccaaggagaggaatgtcatgcaggggacctcctgggtccgcagtgtactgcgagggagcacagatgtccatcccccgctggggtggagagcggcagcaggcctgatggatgagggatcgtggcttcccggcccagagacatgaggtgtccagggccaggccccccaccctcagttggggctgttccgggggtgactgtgagcgatcccactccaaacctgagatggggcagcccgtcctgtgtcctccacagggacaagcagtgggaggagtctgaatggtcaccaggaagcccgggctccatcttgacctcctttttcagggacaggagcaacaggcccctcttccctgactctaagcccttccctgtaaggtgaggcagggtctggagagctctttattggaacagatctggtggttcaaataaacacagtcatgcaaaataaaaaaaaaa

SEQ ID NO: 39 - DNA, 2992 bp, Homo sapiens
  misc_feature: Incyte ID No 2133162CB1
gctgcagctggcagggattgcggggtgccggccgtctgagtttttttaaaactgctcgccgcgaagtctgtctgcagccaaaatgtccaacagaaacaacaacaagcttcccagcaacctgccgcagttacagaatctaatcaagcgagacccgccggcctacatcgaggagtttctacagcagtataatcactacaaatccaatgtggagattttcaaattgcaaccaaataaacccagcaaagaactagcagagctggtgatgtttatggcacagattagtcactgctacccagagtacctaagtaattttcctcaagaggtgaaagatcttctctcctgcaatcataccgtattggatccagatctgcgaatgacattttgcaaagctttgatcttgctgagaaataagaatctcatcaatccatcaagcctgctagaactcttctttgaactttttcgttgccatgataaacttctgcgaaagactttatacacacatattgtgactgatatcaagaatataaatgcaaaacacaagaacaataaagtgaatgtagtattgcaaaatttcatgtacaccatgttaagagatagcaatgcaaccgcagccaagatgtctttagatgtaatgattgaactctacagaaggaacatctggaatgatgcaaaaactgtcaatgttatcacaactgcatgtttctctaaggtcaccaagatattagttgccgctttgacattctttcttgggaaagatgaagatgaaaaacaggacagtgactccgaatctgaggatgatggaccaacagcaagagacctgctagtacaatatgctacagggaagaaaagttccaaaaacaagaaaaagttggaaaaggcaatgaaagtgctcaagaaacaaaaaaagaagaaaaaaccagaggtgtttaacttttcagccattcacttgattcatgatccccaagattttgcggaaaaactactaaagcagcttgagtgctgtaaggagaggtttgaagtgaagatgatgctcatgaaccttatctccagattggtgggaattcatgagcttttcctcttcaatttctatccctttttgcaaaggtttctgcagccccaccaaagagaagtaaccaagatccttctgtttgctgcacaagcatctcatcacctagtacccccagagattattcaatcattgcttatgactgtggcaaacaattttgttaccgacaagaactctggagaagtcatgacagtaggaatcaatgctataaaggagataacagctcgatgtcctctggccatgactgaagaacttctccaagacctggctcagtataaaacacacaaggataagaatgtaatgatgtctgctagaactttgattcacctcttccgaacactgaatcctcagatgctgcagaagaaattccggggtaagcctacagaggcctccatagaagcaagagtacaagaatatggagaattagatgctaaagattacattccaggagcagaagttctggaagttgagaaagaagagaatgctgaaaatgatgaagatggatgggaaagtaccagtctcagtgaggaggaggatgctgatggtgaatggattgatgtgcaacactcttccgatgaagaacagcaagaaatctccaagaagctgaacagcatgcccatggaggagcggaaggccaaagctgcagccatcagcactagccgagttttaactcaggaagacttccagaaaatccgcatggcccaaatgagaaaagaacttgatgctgcccccgggaaatgccagaagaggaaatacattgaaatagacagtgatgaagagcccaggggtgaattactttctcttcgggacattgaacgccttcataaaaagccaaagtctgacaaagagacaagactagcaactgcaatggctggaaagacagaccgaaaagaatttgtgaggaagaaaaccaaaacaaatccattttccagttcgacaaataaagagaagaaaaaacagaagaactttatgatgatgcggtatagccagaatgtccggtcaaaaaataagcgttccttccgagaaaaacagttggcactacgagatgcacttttgaaaaagagaaaaagaatgaagtaacttcctggcaagttttccattcctagaagaatgctaagtttgtgtccttgctctgaaaattggtaaatcaagcatgtttgtttacattaaaaagtccagacacactgtattgtgaaaactgctgaacatgtggcagcaattttgtgtttttattttggagacggctaatggtaggaatgttaatgtaaatagtggtggtaatgtaaaatcatttcatttatcattcatgcaaaaaaaagtatgtattgagtgcctattcattgtcactgtagatgcaaaacgaatgagctgtaaccccttcactcaaggcattgacagcttagctgtgagggtggacacacatatgtgtatttacagtgcagtgtaaatagttctgtagtagaggttaactccatatttctgtgaggtcactgaggccttatggactaactctgtgaggataggagttatatattcttataagacaaaacaaaacaggacaatgttacaagagtaagaggttcttacttgtacataggctttcctgctgaaaacaggcccctgctgtacagattttgggtacataatttagctcttttagtcaatccaagagatttaagtgacccccccccccccgtgttttttttgtttttgtttttgttttgaatgccatgtaaaggctttttggttaagacctcacttttaaaactgccttaagtataaatagtacctttggaatacatttagttcatcatttgagctgccttcatactggtttcctcagccttccttcagcctgtaatattttcagcccactgtttaccttgtctcaataaaaggtttctaatgccaaataaaaaaaaaaaa

SEQ ID NO: 40 - DNA, 850 bp, Homo sapiens
  misc_feature: Incyte ID No 254567CB1
gcgacacgcggtgggcgggtcctgagtcgcgaccctggtccggacctgacctgaattgcgaccccaacctggactgctcccctgaccgcaacccctacccccgcccaccagtatggcccggcacgtgttcctaacggggcccccaggagttggaaaaacaacattgatccataaagccagtgaggttttaaaatcctctggtgtgcctgttgatggattttataccgaagaagtcagacagggagggagaagaataggattcgatgtcgtcacgttgtccggcacccgggggcctttatcgagagttgggttagagcctccacctggaaaacgtgaatgccgagttgggcagtatgtggtcgacctgacttcttttgagcagttggcactacccgtcttgaggaatgccgactgcagcagtggcccagggcaaagagtgtgcgtcatcgatgagattgggaagatggagctcttcagtcagcttttcattcaagctgttcgtcagacgctgtctaccccagggactataatccttggcacaatcccagttcctaaaggaaagccactggctcttgtagaagaaatcagaaacagaaaggatgtgaaggtgtttaatgtcaccaaggaaaacagaaaccaccttctgccagatatcgtgacgtgcgtgcagagcagcaggaagtgaagacacgtgcattcctgccttccgtgaaggagtgcccagttcaagaggagcctgatggagccctgcctgtcgaggctgtatgcctatggggttatggaaccttgtgggcttttctagagaaaactcaacagctgtttcccataaaatgtttaaaagatcaaaaaa

SEQ ID NO: 41 - DNA, 1472 bp, Homo sapiens
  misc_feature: Incyte ID No 3903488CB1
cgcgcgaaggctaagggagtgtggcgggcggctccgggagccaacatgcctcggtatgcgcagctggtcatgggccccgcgggcagcgggaagagcacctactgtgccaccatggtccagcactgtgaagccctcaaccggtctgtccaagttgtaaacctggatccagcagcagaacacttcaactactccgtgatggctgacatccgggaactgatcgaggtggatgatgtaatggaggatgattctctgcgattcggtcccaacggaggattggtattttgcatggagtactttgccaataattttgactggctggagaactgtcttggccatgtagaggacgactatatcctttttgattgtccaggtcagattgagttgtacactcacctgcctgtgatgaaacagctggtccagcagctcgagcagtgggagttccgagtctgtggagtttttcttgttgattctcagttcatggtggagtcattcaagtttatttctggcatcttggcagccctgagtgccatgatctctctagaaattccgcaagtcaacatcatgacaaaaatggatctgctgagtaaaaaagcaaaaaaggaaattgagaaatttttagatccagacatgtattctttattagaagattctacaagtgacttaagaagcaaaaaattcaagaaactgactaaagctatatgtggactgattgatgactacagcatggttcgatttttaccttacgatcagtcagatgaagaaagcatgaacattgtattgcagcatattgattttgccattcaatatggagaagacctagaatttaaagaaccaaaggaacgtgaagatgagtcttcctctatgtttgacgaatattttcaagaatgccaggatgaatgaagagtttactaaaagtaaccatctaaagagcttgtggccaaaccagcagaacattcttctcttcaaaggatgcaatagtagaaagctacttattttaatgaaaaaaagtaaaacttcgttctttatcagcctcatgcctgaatcaaatttttaattattctgaaactgctgctgtttaaagtggaatcttttagtattataacagcatcactttagattttgtaagtcaaaattgaaatgaatgcacatagatttatatataaattagcacctgagctaaggttaaggctggtctaaacttattttcactttttgtattatttttgagatgcaggaattactgtaacaaaatatgtatgtccgaagggaaaaagctgcaaggatatatataagaccactgcttatctgtatcttcccattttcctatattgaaaatgtatattatttatataacttaaaaagtaaaaataactatgttttgagatatgtatgtgtatatataaaagaaacaaaggtttttaatgattcttggacctagataaataatatacattttcctatattgaaaatgt

SEQ ID NO: 42 - DNA, 1561 bp, Homo sapiens
  misc_feature: Incyte ID No 5391816CB1
ccgccccctccgggcttggctctcccaggaggctacgactggagccactggtcccgcaggatccccgcgtcctcggtcgccgcgtccacgtccctctcgcgtccccgcccggcgccacgccgcctcctctgggttcggcctccgcgcggtgcagcgcagtctcaggccgcgggacaagcccgacttaaatctctgcaatggctaacgaagcttatccttgtccgtgtgacattggccacagacttgagtatggagggctaggccgtgaagttcaagtcgagcacatcaaggcttatgtcaccaaatcccccgttgatgcaggcaaagctgtgattgtcattcaagatatatttggctggcagttgcccaataccagatatatagctgacatgatctcaggaaatggatacacaaccattgttccagacttctttgtagggcaagagccttgggacccctctggcgactggtctatcttccctgagtggctgaaaacaagaaatgcccagaagatcgatagagagatcagtgctatcttgaagtatctgaaacaacagtgtcatgcccagaaaattggcatcgtgggattctgctggggtggaactgctgtccatcatttgatgatgaaatactcagaattcagggcaggggtgtccgtctatggcatcgtcaaggattctgaagacatttacaatttaaagaaccccactttgttcatttttgctgaaaatgatgttgtgattccactcaaggacgtatctttgctgactcagaagttgaaagaacactgcaaagttgaatatcaaattaaaacattttctgggcagactcatgggttcgtgcatcggaagagagaagattgctcacctgcagacaagccctacattgacgaggccagaaggaatttaattgagtggctgaacaagtacatgtagcaagaatcaagggcaagccttcctagaatagctttcatcccaaaatttgcttggaaatagttagatcatttgatttaattttcacttttataaaataagtgtaggaatcctaaaattgattatttcatttgaaacacaaattcagtaggacgtaatgcatgaaataatttaatttttgacatgtacatcgaatcataatttaaaaacaaggtctgaccaggtgtagtgcctcatgcctgtaattccagcactttgggaggccaaagtgggtggatcacctgaggtcaggagtttgagaccagcctggccaacatggtgagaccccatctctacaaaaaatacaaaaattagcctggtgtggtggtgcacacctgtagtcccagctacttgggaggctgaggcacaagaatcaatagaacccaggaggtggagactgcagtgagccaagattgtgccactactgtactctagcctgggcagcagagtgagaccctgtctcaaaaataaataagtaaataaataaataaataaaataaaaaccaggtcagtacctggagaatttgaatgatagagaatgatagagtaataccctaattattagttaaaacctacaggccgggtgtggt

SEQ ID NO: 43 - DNA, 1949 bp, Homo sapiens
  misc_feature: Incyte ID No 5886989CB1
gccctggccgcggctgctacgcggggctgccaccgccccgggcgacccaatttcgcggcctagtggggcgtacgggcctcttttgaaagcctgagttacgatgtattgagcgcgtcgtatgcggccagcactaaggtccttctggcactcctctggtggaccgcccccatcggccacacttgccctgctctccagtgattctgtagctcctggctccgtagtctcgcggacagctctctttcctggaagatgtttctggtgggcctgacagggggcattgcctcaggcaagagctcagtgatccaggtgttccagcagctgggctgtgcggtgattgacgtggacgtgatggcccggcacgtcgtgcagccaggataccctgcccaccggcgcatcgtagaggtcttcggcactgaggtcttgctggagaacggcgacataaatcgcaaggtcctgggggacctgatctttaaccagcctgaccggcggcagctgctcaacgccatcacccaccccgagattcgcaaggagatgatgaaggagacgttcaagtacttcctccggggataccgctacgtgattctggatatccccctgctgtttgagaccaagaagttgctcaagtacatgaagcacaccgtggtagtatactgcgaccgggacacacagctggcacggctgatgcggcggaacagcctgaaccgcaaggacgcagaggcccgcatcaatgcccagctgcccctgacagacaaggcccgcatggcccgccatgtcctagacaactcgggcgagtggagtgtcaccaaacgccaggtcatcctcttgcacactgagctggagcgctccctggagtacctgccgctgaggtttggggtcctcacagggctcgctgccattgccagcctcctctacctgctcacccactaccttctgccttacgcctagtggggcactcaaggcagggagccccaggcctccatctatctccttggaggctgaagccaggtaacacatcctgtttcctctcagccccctccacacacacacacacacacacacacacacacacacacacacacacggactctccgaatcagggctgggcctctgtgccaggccatcccgtctttggagtgtatcctatttgaggtcaagaaatagcctgctgtccttcccaccttccctgagcagcacctacagaacagcaaccgtgatggtgtgtgaaagcaacagtcagagactgctatacatttctgtgggtgggggtagggaatactggctctgtggccctcctcagagctgggaggccaagggttcactgggcttgaatcctctctaagcacctaccccattatggcctaaacctcctcagtggaccacgggaaatcatgttggaccaagggtttccatgttcaaacagtcaacaaggccctcccctaggttgagatatttttgatactgaaggcccaggccccccagtatggcttgctgccagcaagcagcaaggattctctcgtgggtaacgctgtagccaggcaggaccagcctcttctgggagacccctctccccacgtagggtttgtgtagtgctcccacatcctgcttattgcctgccacccctgcttctcgcctggacctcttggtattccgtgtacaccatccttgctgtttcttgcctctgtgccttcaacctgctgctcctggcctgggataccttttattttcttttatttcttcttcctttttttttttttttttttttttttaacctacaactagctctcagttcaggcactgtctaaagccccaggctgggttaggtggtctaggataccaactccccctcaacattttaccttaatccaacagtacagtaattagccgaaacttgcctggtttttcctactagactgtaagccctctagggacagggacagtgtcttattcat

SEQ ID NO: 44 - DNA, 2221 bp, Homo sapiens
  misc_feature: Incyte ID No 723432CB1
ccgggacacgtggtacggaaccggcgccgcgcttgctgctggtaacagggccttgcctagtgggccttccttcccaggtcgcccctcagtctccactagagacaggactgaccagttgctcttccttccaagaaccttcgagatctgcggtctggggtctggttgaaagatggcggccctcactaccctgtttaagtacatagatgaaaatcaggatcgctacattaagaaactcgcaaaatgggtggctatccagagtgtgtctgcgtggccggagaagagaggcgaaatcaggaggatgatggaagttgctgctgcagatgttaagcagttggggggctctgtggaactggtggatatcggaaaacaaaagctccctgatggctcggagatcccgctccctcctattctgctcggcaggctgggctccgacccacagaagaagaccgtgtgcatttacgggcacctggatgtgcagcctgcagccctggaggacggctgggacagcgagcccttcaccctggtggagcgagacggcaagctgcatgggagaggttcgactgatgataagggcccggtggccggctggataaacgccctggaagcgtatcagaaaacaggccaggagattcctgtcaacgtccgattctgcctcgaaggcatggaggagtcaggctctgagggcctagacgagctgatttttgcccggaaagacacattctttaaggatgtggactacgtctgcatttctgacaattactggctgggaaagaagaagccctgcatcacctacggcctcaggggcatttgctactttttcatcgaggtggagtgcagcaacaaagacctccattctggggtgtacgggggctcggtgcatgaggccatgactgatctcattttgctgatgggctctttggtggacaagagggggaacatcctgatccccggcattaacgaggccgtggccgccgtcacggaagaggagcacaagctgtacgacgacatcgactttgacatagaggagtttgccaaggatgtgggggcgcagatcctcctgcacagccacaagaaagacatcctcatgcaccgatggcggtacccgtctctgtccctccatggcatcgaaggcgccttctctgggtctggggccaagaccgtgattcccaggaaggtggttggcaagttctccatcaggctcgtgccgaacatgactcctgaagtcgtcggcgagcaggtcacaagctacctaactaagaagtttgctgaactacgcagccccaatgagttcaaggtgtacatgggccacggtgggaagccctgggtctccgacttcagtcaccctcattacctggctgggagaagagccatgaagacagtttttggtgttgagccagacttgaccagggaaggcggcagtattcccgtgaccttgacctttcaggaggccacgggcaagaacgtcatgctgctgcctgtggggtcagcggatgacggagcccactcccagaatgaaaagctcaacaggtataactacatagagggaaccaagatgctggccgcgtacctgtatgaggtctcccagctgaaggactaggccaagccctctgtgtgccatctccaatgagaaggaatcctgccctcacctcacccttttccaacttgcccagggaagtggaggttccctctttcctttccctcttgtcaggtcatccatgactttagagaacagacacaagtgtatccagctgtccacgggtggagctacccgttgggcttatgagtgacctggagtgacagctgagtcaccctgggtaagttctcagagtggtcaggatggcttgacctgcagaagatacccaaggtccaaaagcacaaggtctgcggaaagttctggttgtcggctgggcaccacggctcacacctataatcgagcactttgggaggccaagacaggaggatcacttgaggccaggagtctgagacaagcctaggcaacaaaacaagactctgtctctacaaaaagtttaagaaatgagccagacatggtggtgtatgcctgtagtcccagccactcagaaggctgaggcaggaggatcgcttgagaccaagagtttgagcctgcggtgagctgtgaatgcacacggcactcaagcctgggcaatgtagcaagatcctgtctctacaagaagattttttaaagatgagccaagtgtggtgg

SEQ ID NO: 45 - DNA, 1619 bp, Homo sapiens
  misc_feature: Incyte ID No 1332963CB1
tgcagctggcgaagttgggcgactggcggatgcaggccttgcggcacgtcgtgtgcgccctgtccggcggcgtggacagcgccgtggccgcgctgctgctgaggcggagaggttaccaggtgacaggggtgtttatgaagaactgggactcactggatgaacatggggtctgtactgccgacaaagactgtgaagatgcttacagagtttgccagatcttagacatccctttccatcaagtgtcctacgtaaaggagtattggaatgatgtgttcagtgactttttgaatgagtatgaaaaaggaaggactcccaatcctgacatagtttgcaacaagcacatcaaatttagttgcttttttcattatgctgtggataatcttggggcagatgccattgccacaggtcactatgcaagaacttccctggaagatgaagaagtctttgagcagaagcacgttaagaagcccgaagggcttttcagaaatcggtttgaagttagaaatgcggtaaaactcctccaggcagctgacagctttaaagaccagaccttctttctcagccaggtttcccaggatgccctgaggagaaccatcttccctctggggggattaacgaaagagtttgtaaagaaaatcgctgctgagaatagacttcatcatgtgcttcagaagaaagagagcatgggcatgtgtttcatcgggaagaggaattttgaacatttccttcttcagtatctgcagcctcgacctggtcactttatttccatagaagacaataaggttctgggaacacataaaggttggttcctgtataccttgggccagagagcaaacataggtggcctgagagagccctggtacgtggtggagaaggacagcgtcaagggtgacgtgtttgtggccccccggacagaccacccagccctgtacagggacctgctgaggaccagccgcgtgcactggattgcggaggagcctcccgcagcactggtccgggacaagatgatggagtgccacttccgattccgccaccagatggcactagtgccctgtgtgctgaccctcaatcaagatggcaccgtgtgggtgacagctgtgcaggctgtgcgtgcccttgccacaggacagtttgctgtgttctacaagggggacgagtgcctgggcagcgggaagatcctgcggctggggccgtctgcctacacgctccagaagggccagcgcagagctgggatggccactgagagccccagtgacagcccagaagatggtccaggcctgagtcccttgctctgacagagatggatctgctagaaggaacctggagagcaggacccatggctgggcggctggtgagcagtccaggtgcccaagggccagcttgctgctgcccaaagcagaggaagccgggctggctgagggtccgaaaagcctgcaggggcccggcgagccccaggaagagcctcagctccaggctggggctctggctgctggagcatctgctggctggtggggtggcccgagttccccttcaccgcccccagggagggtttcccacctcagagtacaccgaggggacctgcagagggggctgtcgggacagcgtggaataaacataag

SEQ ID NO: 46 - DNA, 1448 bp, Homo sapiens
  misc_feature: Incyte ID No 1559410CB1
gctgtccggaagtcgagttagtctagttagtatcggcctgttatctccttttgcgcgacacggtctcagctgttccgcctgaggcgagtgacgctggccgccaacgaggtatacgtactgggaccctcgccctcagtctcgtctccggcgcggctacctgccccgttttccctgtgagttgacctgctccgggccgcgggccgccaatggcaggggccgctccgaccacggccttcgggcaggcggtgatcggcccgccgggctcagggaagaccacgtactgcctgggcatgagtgagttcctgcgcgcgctgggccggcgcgtggcggtggtgaacctggacccggccaacgaggggctgccgtacgagtgtgccgtggacgtgggcgagctggtggggctgggcgacgtgatggacgcgctgcgcctggggcccaacggcggcctgctctactgcatggagtacctggaagccaacctggactggctgcgtgccaagctcgaccccctccgcggccactacttcctcttcgactgcccaggccaggtggagctctgcacgcatcacggcgccttgcgcagcatcttctcccaaatggcgcagtgggacctcaggctgactgccgtccacctcgtggattctcactactgcacagaccctgccaagttcatttcagtactgtgtacctccctggccaccatgctgcacgtggaactgccccacatcaacctcctttccaagatggacctcattgagcattatgggaagctggccttcaacctggactactacacagaggttctggacctctcctacctgcttgaccacctggcttctgaccctttcttccgccactaccgccagctcaatgagaagctagtgcagctcatcgaagactatagccttgtctcctttatccctctcaacatccaggacaaggagagcatccagcgagtcctgcaggctgtggataaagccaatggatactgtttcggagcccaagagcagcgaagcttggaagccatgatgtctgccgcaatgggagccgacttccatttctcttccacactgggcatccaggagaagtacctggcaccctcgaaccagtcagtggagcaggaagccatgcagctgtagcaacaaggtggaccctggagagcaggatgcataatccagcactggggaaagtggaggctcctgatgcaggctgcagacccaagagcaagtcctcccagccagagctggcgggctggcaaggggatattcagctctgcaaaggacttctggccaaaaagccagacatggtgccaagcagaacaccccccatactgtcagtggtgtccgtgagctctgggccctgccaccagaaagtcgagcactggtcctagtcaggctgtgatgaaatgtgctacaatacaagagtttattttctaaaaaaaaaaaaaa

SEQ ID NO: 47 - DNA, 2225 bp, Homo sapiens
  misc_feature: Incyte ID No 1752587CB1
agagagagagagagagaactagtctcgagccatcatggaagcaatgtggctcctgtgtgtggcgttggcggtcttggcatggggcttcctctgggtttgggactcctcagaacgaatgaagagtcgggagcagggaggacggctgggagccgaaagccggaccctgctggtcatagcgcaccctgacgatgaagccatgttttttgctcccacagtgctaggcttggcccgcctaaggcactgggtgtacctgctttgcttctctgcaggaaattactacaatcaaggagagactcgtaagaaagaacttttgcagagctgtgatgttttggggattccactctccagtgtaatgattattgacaacagggatttcccagatgacccaggcatgcagtgggacacagagcacgtggccagagtcctccttcagcacatagaagtgaatggcatcaatctggtggtgactttcgatgcagggggagtaagtggccacagcaatcacattgctctgtatgcagctgtgagggccctgcactcagaagggaagttacctaaaggtaaggcttgttccttttgcaaagggccacaagatactgtccctctgagaaacttatgagggggaaatttgcaaatccacagagagctgccctcagccgagcagaggcagtggttggggaggccgccacactcttcctgactcacctcaaggtacctggcaggttgtatctaccagcaagtgcctgcctgcagggccacctctggatgagtggggaggccaggtttgcccctcaacctgtctagctttttcacagggtggatgttttacacctctggtattcttccagtctgaactcaatcccatcccccaaccaattaggcaggttgtaaatgactgctgggtgcttgatgaaccaatctgcctgagttaggaactcttcctaacccacataaagtatttgaccctacctgaccatttactgcatgacccccatgcttagcctctctggaccagtttctggtcagtcatacctcaccggcgcaatacgaagattcaatgaatgaaaacgtaagtagtttgtgccctttctatgtgggatgactgaaaggagcttaccaagccatgatgactttggaacatgactatagtttgtttgattcttatatcaatcctgtgagacaggcaggtctcacactatcaacaccaatttacagattagaaaactgagactgagagtggttaagggactttgccaagtctgtcaattcttgactgacagagctggaaccaggtctctaaactcccaatacagtaattcctgtctcctttacttcctttggctttgaggtagccagatctcaacctctagcagccaacctttttctggtagggcacaccgcaggtagagggtagccagccaggtcttctatggcccttttactcgttctagctgcaagaatcatgggcacagggcagctttagtgccacagaaacacgtggcaatgctgtggccactgtcctgccctgccctgagccacagggtagagggaggatgctcaggggaaaatctggctggaggcctcaaggcttatcacttcaccctgtctcctctccatccagggtgctctgtgctcacgcttcagtctgtgaatgtgctgcgcaagtacatctcccttctggatctgcccttgtctctgcttcatacgcaggatgtcctcttcgtgctcaacagcaaagaagtggcacaggccaagaaagccatgtcctgccaccgcagccagctcctctggttccgccgcctctacattatcttctcccggtacatgagaatcaactcactgagcttcctctgaagccttgaagggttttcaggtccaaggaacaaaggggaaaatagacaaaggagtgcagaggacctggcctggcactggcttatttacctgagctcaaggagatccccgctggagcagcctctgcaaaagggagcccatgtaggccaggggctgtccaaactccagcttcttcccctgggaaaaaacccaaagaaccaaaaacaaaccaccccaaggataataatagctacactgctagcttctcaagttcttgtgaaaaacaatttacataatgacacagtagatgtggaacacctagcccagtgcctgggcaggtccctattatcataaatgaacataaaagtgctctaaaaacactcaaaaaaaaaaaa

SEQ ID NO: 48 - DNA, 1400 bp, Homo sapiens
  misc_feature: Incyte ID No 1911509CB1
gcgccagcccggggcggcccagtcggcctgtcagccggcttcgagataagtcccggcgcttgcgcggcggcggctatggcggcggaggaggaggaggtggactctgccgacaccggagagaggtcaggatggctaactggttggctccccacatggtgccctacgtctatatcacaccttaaagaagctgaagagaagatgttaaaatgtgtgccttgcacatacaaaaaagaacctgttcgtatatctaatggaaataaaatatggacactgaagttctctcataatatttcaaataagactccacttgtccttctccatggttttggaggaggtcttgggctctgggcactgaattttggagatctttgcaccaacagacctgtctatgcttttgacctattgggttttggacgaagtagtagacccaggtttgacagtgatgcagaagaagtggagaatcagtttgtggaatccattgaagagtggagatgtgccctaggattggacaaaatgatcttgcttgggcacaacctaggtggattcttggctgctgcttactcgctgaagtacccatcaagggttaatcatctcattttagtggagccttggggtttccctgaacgaccagaccttgctgatcaagacagaccaattccagtttggatcagagccttgggagcagcattgactccctttaaccctttagctggcctaaggattgcaggaccctttggtttaagtctagtgcagcgtttaaggcctgatttcaaacgaaagtattcttcaatgttcgaagacgatactgtgacagaatacatctaccactgtaatgtgcagactccaagtggtgagacagctttcaagaatatgactattccttatggatgggcaaaaaggccaatgctccagcgaattggtaaaatgcaccctgacattccagtttcagtgatctttggcgcccgatcctgcatagatggcaattctggcaccagcatccagtccttacgaccacattcatatgtgaagacaatagctattcttggggcaggacattatgtatatgcagatcaaccagaagaattcaaccagaaagtaaaggagatctgcgacactgtggactgaacacactgaagctctgatgggaaaacctggtgactgatatagttgttcagcaataattcatagtctgtgatgaagagtagtgaatacaacacacaaccaggcagccttcttgactatactttgcacatgttttctttaggaattcactcacacatttaaaccagttagtgccttctagaagaatggctttcctttctcctacacaaaattgaaatatacaagtctctaaatataatacctttaaataaaaggttatttgtccctctgaaaaaaaa

SEQ ID NO: 49 - DNA, 1361 bp, Homo sapiens
  misc_feature: Incyte ID No 2210170CB1
ccgcgacagtttcccagcagggctcacagcagcgttccgcgtcatggggatttggcagcgtctgctgctttttggtggggtgtcgctccgggctggtggcggggccactgccccgcttgggggaagccgagcgatggtttgtgggcgccagttgtctggcgccgggagtgagaccctaaaacaaagaagaacacaaatcatgtcccgaggacttccaaagcagaaaccgatagaaggtgttaaacaagttatagttgtggcttctggaaagggtggagtcggaaaatctactacagcagtgaatcttgcacttgcactagcagcgaacgattcgtccaaggccattggtttgctagatgtggatgtgtatggaccttcagttccaaagatgatgaatctgaaaggaaatccggaattatcacagagcaacctaatgaggcctctcttgaattatggtattgcttgtatgtctatgggctttctggttgaagaaagtgaaccagtagtttggagaggccttatggtaatgtcggccattgagaaattgttgaggcaggtagattggggtcaactggactacttagttgtagacatgccaccaggaactggagatgtgcagttatcagtctcacagaatattcctataacaggtgctgtgattgtctccacgccccaggacatcgcattgatggatgcacacaagggtgctgagatgtttcgcagagtccacgtgcccgtaagcgtttacagcttcactgtgaaaaatataaaactcttctaactgaagaagcaagtcattgaaaaatacagttgaagttttaatttagtcctgtgcagaagtctttatgaaagtcctagtttatcattttggtgactttaaaaacacacttataggtacaaacatgtctatacatgatattaatccataagaattgtaaaacaccctttaggtaagagttttctatagaagtactacttctctcatacttgcagctatcactgacaaaaaaaaattacaagagtgttttaggaaattatttagatagataggcttgaatagatcacttatggggaagcggatgaaaaaaagattacttttttccttctgcctcctacagttcaactcatttatagctcacattcttctaggagacacttcaggatcttctccctaatttggtggaatgaaagttaaaaataagataaaaatagacaaaataattaaaattagatggggttagaagtgaagtcagtaaacaaattgcctgctattaagaaacttaaatacatgctaggggtgtaccgtgacttttctagcagccaaaggaaaagaaaaatagtacgcaatataaaacattgcc

SEQ ID NO: 50 - DNA, 993 bp, Homo sapiens
  misc_feature: Incyte ID No 234664CB1
gaagggccggcggctctggctgcccggcggttgagagcatggcctctccaggggcaggtagggcgcctccggagttaccggagcggaactgcgggtaccgcgaagtcgagtactgggatcagcgctaccaaggcgcagccgattctgccccctacgattggttcggggacttctcctccttccgtgccctcctagagccggagctgcggcccgaggaccgtatccttgtgctaggttgcgggaacagtgccctgagctacgagctgttcctcggaggcttccctaatgtgaccagtgtggactactcatcagtcgtggtggctgccatgcaggctcgctatgcccatgtgccgcagctgcgctgggagaccatggatgtgcggaagctggacttccccagtgcttcttttgatgtggtgctcgagaagggcacgctggatgccctgctggctggggaacgagatccctggaccgtgtcctctgaaggtgtccacactgtggaccaggtgttgagtgaggtgagccgcgtgcttgtccctggaggccggtttatctcaatgacttctgctgccccccactttcggaccagacactatgcccaagcctattatggctggtccctgaggcatgctacctatggcagcggtttccacttccatctctacctcatgcacaagggcgggaagctcagtgtggcccagctggctctgggggcccaaatcctctcaccccccagacctcccacctcaccttgcttccttcaggactcagatcatgaggacttccttagtgccattcagctctgaggccagagcatggtcctccacccttcctgccattctgccctgggctcctcaggtagttggaattcctgacttaggacttggggttgggtccaaggtgcttacatcccaggggcctcatgcctaagatagagggtgggagcgaacccacatgaaccaatacagcccagctccaactagaaaaaaaaaa

SEQ ID NO: 51 - DNA, 1367 bp, Homo sapiens
  misc_feature: Incyte ID No 2884114CB1
ctggaaccctggccgagtccgaaaaaagccagatctggaaggtggctgcggaacggttttaagcggaagatggaggagccggaggaaccggcggacagtgggcagtcgctggtcccggtttatatctatagtcccgagtatgtcagtatgtgtgactccctggccaagatccccaaacgggccagtatggtgcattctttgattgaagcatatgcactgcataagcagatgaggatagttaagcctaaagtggcctccatggaggagatggccaccttccacactgatgcttatctgcagcatctccagaaggtcagccaagagggcgatgatgatcatccggactccatagaatatgggctaggttatgactgcccagccactgaagggatatttgactatgcagcagctataggaggggctacgatcacagctgcccaatgcctgattgacggaatgtgcaaagtagcaattaactggtctggagggtggcatcatgcaaagaaagatgaagcatctggtttttgttatctcaatgatgctgtcctgggaatattaagattgcgacggaaatttgagcgtattctctacgtggatttggatctgcaccatggagatggtgtagaagacgcattcagtttcacctccaaagtcatgaccgtgtccctgcacaaattctccccaggatttttcccaggaacaggtgacgtgtctgatgttggcctagggaagggacggtactacagtgtaaatgtgcccattcaggatggcatacaagatgaaaaatattaccagatctgtgaaagtgtactaaaggaagtataccaagcctttaatcccaaagcagtggtcttacagctgggagctgacacaatagctggggatcccatgtgctcctttaacatgactccagtgggaattggcaagtgtcttaagtacatccttcaatggcagttggcaacactcattttgggaggaggaggctataaccttgccaacacggctcgatgctggacatacttgaccggggtcatcctagggaaaacactatcctctgagatcccagatcatgagtttttcacagcatatggtcctgattatgtgctggaaatcacgccaagctgccggccagaccgcaatgagccccaccgaatccaacaaatcctcaactacatcaaagggaatctgaagcatgtggtctagttgacagaaagagatcaggtttccagagctgaggagtggtgcctataatgaagacagcgtgtttatgcaagcagtttgtggaatttgtgactgcagggaaaatttgaaagaaattacttcctgaaaatttccaaggggcatcaagtggcagctggcttcctggg

SEQ ID NO: 52 - DNA, 1317 bp, Homo sapiens
  misc_feature: Incyte ID No 4103559CB1
actagggtcctagcacagtgtctgatggagctttcctaccagaccctgaaattcacgcatcaggcgcgggaagcgtgcgagatgaggacagaagcacgacgaaaaaatcttctcattttgatttcgcattatttaacacaagaagggtatatcgatacagcaaatgctttggagcaagaaactaaactggggttacgacggtttgaagtttgtgacaacattgatcttgaaactattttgatggaatatgagagttattattttgtaaaatttcagaaataccccaaaattgtcaaaaagtcatcagacacagaaaataatttaccgcaaagatgtagagggaagaccagaaggatgatgaacgacagttgtcaaaatcttcccaagatcaatcagcagaggccccggtccaaaaccacagcggggaagacaggggacaccaaatcgctcaataaggagcatcctaatcaggaggtagttgataacactcgcctggaaagtgccaacttcggcctacatatatcaagaatccgtaaagacagtggagaggaaaatgcccacccacgaagaggccaaatcattgacttccaagggctgctcacagatgccatcaagggagcaaccagtgaacttgccttgaacaccttcgaccataatccagacccctcagaacgactgctgaaacctctgagtgcatttattggcatgaacagtgagatgcgagaattggcagccgtggtgagccgggacatttatctccataatccaaacataaagtggaatgacattattggacttgatgcagccaagcagttagtcaaagaagctgttgtgtatcctataaggtatccacagctatttacaggaattctttctccctggaaaggactactgctgtacggccctccaggtacaggaaagactttactggccaaagctgtggccactgaatgtaaaacaaccttctttaacatttctgcatccaccattgtcagcaaatggagaggggattcagaaaaactcgttcgggtgttatttgagcttgcccgctaccacgccccatccacgatcttcctggacgagctggagtcggtgatgagtcagagaggcacagcttctgggggagaacatgaaggaagcctgcggatgaagacagagttactggtgcagatggatgggctggcacgctcagaagatctcgtatttgtcttagcagcttctaacctgccgtggtaagagaccaagagagtaaattttgaatacattttcaggagtcactaagtgcaaataaaaattttatattgaccacttcaaaaa